(19) United States
(12) Patent Application Publication
(10) Pub. No.: US 2019/0231888 A1
(43) Pub. Date: Aug. 1, 2019

(54) TEXAPHYRIN AND ANTITUMOR ANTIBIOTIC CONJUGATES

(71) Applicant: BOARD OF REGENTS, THE UNIVERSITY OF TEXAS SYSTEM, Austin, TX (US)

(72) Inventors: Jonathan L. SESSLER, Austin, TX (US); Min Hee LEE, Seoul (KR)

(21) Appl. No.: 16/338,992

(22) PCT Filed: Oct. 3, 2017
(86) PCT No.: PCT/US2017/054919

§ 371 Date: Apr. 2, 2019

Related U.S. Application Data
(60) Provisional application No. 62/403,339, filed on Oct. 3, 2016.

(51) Int. Cl. A61K 47/54; A61K 31/65; A61K 9/00; A61K 47/69; A61K 45/06; A61K 49/08; A61K 49/00; A61P 35/04

(52) U.S. Cl. CPC A61K 47/546; A61K 31/65; A61K 9/0019; A61P 35/04; A61K 45/06; A61K 49/085; A61K 49/0052; A61K 47/6911

(57) ABSTRACT

The present disclosure relates to texaphyrin compounds linked with an antitumor antibiotic such as an anthcyanine antitumor antibiotic such as doxorubicin and danurubicin. The texaphyrin and the antitumor antibiotic are joined together by a group which is cleavable in vivo and results in increased activity and deliverance of the cytotoxic compound to target cells. Also provided herein are pharmaceutical compositions and methods of use thereof.

This application claims the benefit of priority to U.S. Provisional Application No. 62/403,339, filed on Oct. 3, 2016, the entirety of which is incorporated herein by reference.

This invention was made with government support under Grant No. R01 CA068682 awarded by the National Institutes of Health. The government has certain rights in the invention.

### BACKGROUND OF THE INVENTION

### 1. Field of the Invention

The present disclosure relates generally to the fields of medicine, imaging agents, and therapeutic agents. The present disclosure relates to texaphyrin compounds conjugated to antitumor antibiotics such as doxorubicin.

### 2. Description of Related Art

The early diagnosis and accurate characterization of cancerous lesions is crucial to determining the prognosis of the patient and making proper therapeutic decisions. Magnetic resonance imaging (MRI) is a particularly useful noninvasive technique that is widely used to visualize and evaluate hepatic metastases (Namasivayam et al., 2007). Cancer specific MR contrast agents, such as liver specific agents, based on gadolinium chelates have been developed that are capable of providing the enhanced lesion-to-healthy tissue images (Semelka et al., 1999; Schima et al., 2005). Recently, fluorescence imaging using small molecules, such as indocyanine green (ICG), has been applied to cancer visualization and fluorescence-guided surgery (Kokudo and Ishizawa, 2012; Shimada et al., 2015). MR imaging provides good spatial resolution and soft tissue contrast, while fluorescence imaging is characterized by high sensitivity and provides valuable information on the local cellular level. The combination of MR and fluorescence imaging could provide synergistic advantages over either modality alone. Particularly attractive would be agents that permit diagnosis via both these modalities while also delivering a therapeutic agent preferentially to the desired cancerous tissues.

Theranostics are systems that permit diagnostic imaging while providing a potential therapeutic benefit. This is a very active area of research and currently, theranostics are being developed for use in a number of disease targets (Vivero-Escoto et al., 2012; Shanmugam et al., 2014; Bardhan et al., 2011). Many efforts have centered on the development of cleavable linker-based multifunctional conjugates for targeted cancer drug delivery and fluorescence-based imaging (Kumar et al., 2015; Lee et al., 2015; Lee, et al., 2012). Many of the theranostics that have been developed contain of a fluorescent reporter, a cleavable linker, a prodrug, and a tumor guiding ligand. The cleavable linker is typically chosen to undergo scission upon exposure to the cancer environment (high levels of biomolecules present in cancer cells; relatively low pH, etc.). This releases the active drug agent and, in the case of the most effective systems, leads to a readily visualized enhancement in the fluorescence emission intensity. The use of motexafin gadolinium (MGd) to create new theranostic agents is attractive because it permits localization to be monitored by MRI imaging. However, the paramagnetic nature of the coordinated Gd³⁺ center has so far precluded the use of MGd-derived systems for fluorescence-based imaging. The ability of MGd to localize preferentially to cancerous lesions has been validated in both clinical models and preclinical studies through inter alia magnetic Gd³⁺ T₁-enhanced MRI and tissue-specific HPLC analyses Preihs et al., 2013; Young et al., 1996; Sessler et al., 1993; Mehta et al., 2009; Patel et al., 2008; Wei et al., 2005). Therefore, there remains a need to develop theranostic systems which contain MGd and an active therapeutic agent that allows for imaging via at least two methods (MRI and optical) as well as delivers a therapeutic agent.

### SUMMARY OF THE INVENTION

In some aspects, the present disclosure provides compounds

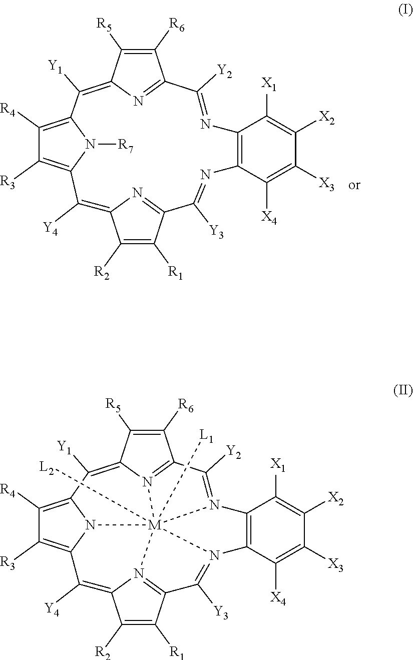

wherein:

Y₁-Y₄ are each independently selected from: hydrogen, amino, cyano, halo, hydroxy, or hydroxyamino,
alkyl_{(C≤12)}, cycloalkyl_{(C≤12)}, alkenyl_{(C≤12)}, cycloalkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, alkoxy_{(C—12)}, acyloxy_{(C≤12)}, aryloxy_{(C≤12)}, heteroaryloxy_{(C≤12)}, heterocycloalkoxy_{(C≤12)}, amido_{(C≤12)}, alkylamino_{(C≤12)}, dialkylamino_{(C≤12)}, alkylthio_{(C≤12)}, arylthio_{(C≤12)}, alkylsulfinyl_{(C≤12)}, arylsulfinyl_{(C≤12)}, alkylsulfonyl_{(C≤12)}, arylsulfonyl_{(C≤12)}, or a substituted version of any of these groups; or

R₁-R₆ are each independently selected from: hydrogen, amino, cyano, halo, hydroxy, hydroxyamino, or nitro,
alkyl_{(C≤12)}, cycloalkyl_{(C≤12)}, alkenyl_{(C≤12)}, cycloalkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, alkoxy_{(C≤12)}, acyloxy_{(C≤12)}, aryloxy_{(C≤12)}, heteroaryloxy_{(C≤12)}, heterocycloalkoxy_{(C≤12)}, amido_{(C≤12)}, alkylamino_{(C≤12)}, dialkylamino_{(C≤12)}, or a substituted version of any of these groups; or
a PEG moiety wherein the PEG moiety is of the formula: —(OCH₂CH₂)ₚOR₉; wherein:
   p is 1-20; and
   R₉ is hydrogen, alkyl_{(C≤8)}, or substituted alkyl_{(C≤8)}; or
an antitumor antibiotic linked through a cleavable covalent linker;

R₇ is hydrogen,
alkyl_{(C≤8)}, cycloalkyl_{(C≤8)}, alkenyl_{(C≤8)}, cycloalkenyl_{(C≤8)}, alkynyl_{(C≤8)}, alkoxy_{(C≤8)}, or a substituted version of any of these groups, or an amino protecting group;

X₁-X₄ are each independently selected from: hydrogen, amino, cyano, halo, hydroxy, hydroxyamino, or nitro,
alkyl_{(C≤12)}, cycloalkyl_{(C≤12)}, alkenyl_{(C≤12)}, cycloalkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, alkoxy_{(C≤12)}, acyloxy_{(C≤12)}, aryloxy_{(C≤12)}, heteroaryloxy_{(C≤12)}, heterocy cloalkoxy_{(C≤12)}, amido_{(C≤12)}, alkylamino_{(C≤12)}, dialkylamino_{(C≤12)}, or a substituted version of any of these groups; or
a PEG moiety wherein the PEG moiety is of the formula: —(OCH₂CH₂)ₙOR₈; wherein:
   n is 1-20; and
   R₈ is hydrogen, alkyl_{(C≤8)}, or substituted alkyl_{(C≤8)};

L₁ and L₂ are each independently absent, a neutral ligand, or an anionic ligand; and

M is a metal ion;

or a pharmaceutically acceptable salt thereof.

The compounds may be further defined as:

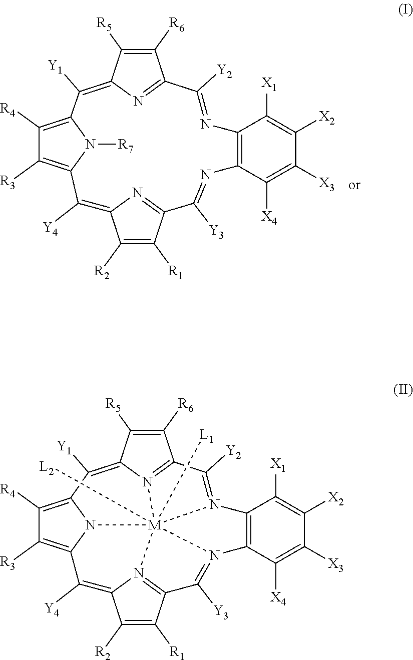

wherein:

Y₁ and Y₄ are each independently selected from: hydrogen, amino, cyano, halo, hydroxy, or hydroxyamino,
alkyl_{(C≤12)}, cycloalkyl_{(C≤12)}, alkenyl_{(C≤12)}, cycloalkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, alkoxy_{(C≤12)}, acyloxy_{(C≤12)}, aryloxy_{(C≤12)}, heteroaryloxy_{(C≤12)}, heterocycloalkoxy_{(C≤12)}, amido_{(C≤12)}, alkylamino_{(C≤12)}, dialkylamino_{(C≤12)}, alkylthio_{(C≤12)}, arylthio_{(C≤12)}, alkylsulfinyl_{(C≤12)}, arylsulfinyl_{(C≤12)}, alkylsulfonyl_{(C≤12)}, arylsulfonyl_{(C≤12)}, or a substituted version of any of these groups;

Y₂ and Y₃ are each independently selected from hydrogen, alkyl_{(C1-6)}, or substituted alkyl_{(C1-6)};

R₁-R₆ are each independently selected from: hydrogen, amino, cyano, halo, hydroxy, hydroxyamino, or nitro,
alkyl_{(C≤12)}, cycloalkyl_{(C≤12)}, alkenyl_{(C≤12)}, cycloalkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, alkoxy_{(C≤12)}, acyloxy_{(C≤12)}, aryloxy_{(C≤12)}, heteroaryloxy_{(C≤12)}, heterocycloalkoxy_{(C≤12)}, amido_{(C≤12)}, alkylamino_{(C≤12)}, dialkylamino_{(C≤12)}, or a substituted version of any of these groups; or
a PEG moiety wherein the PEG moiety is of the formula: —(OCH₂CH₂)ₚOR₉;
   wherein:
   p is 1-20; and
   R₉ is hydrogen, alkyl_{(C≤8)}, or substituted alkyl_{(C≤8)}; or
an antitumor antibiotic linked through a cleavable covalent linker;

R₇ is hydrogen,
alkyl_{(C≤8)}, cycloalkyl_{(C≤8)}, alkenyl_{(C≤8)}, cycloalkenyl_{(C≤8)}, alkynyl_{(C≤8)}, alkoxy_{(C≤8)}, or a substituted version of any of these groups, or an amino protecting group;

X₁ and X₄ are each independently selected from: hydrogen, fluoride, alkyl_{(C1-6)}, or substituted alkyl_{(C1-6)}; or

X₂ and X₃ are each independently selected from: a PEG moiety wherein the PEG moiety is of the formula: —(OCH₂CH₂)ₙOR₈; wherein:
n is 1-20; and
R₈ is hydrogen, alkyl_{(C≤8}), or substituted alkyl_{(C≤8)};

L₁ and L₂ are each independently absent, a neutral ligand, or an anionic ligand; and

M is a metal ion;

or a pharmaceutically acceptable salt thereof.

In some embodiments, the compounds are further defined as:

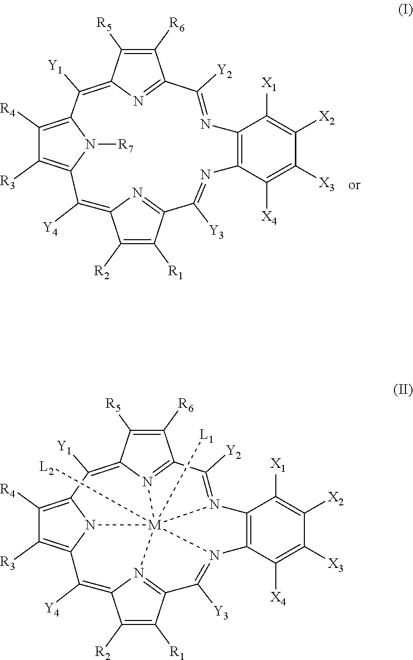

wherein:

Y₁-Y₄ are each independently selected from hydrogen, alkyl_{(C1-6)}, or substituted alkyl_{(C1-6)};

R₁-R₆ are each independently selected from: hydrogen, amino, cyano, halo, hydroxy, hydroxyamino, or nitro,
alkyl_{(C≤12)}, cycloalkyl_{(C≤12)}, alkenyl_{(C≤12)}, cycloalkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, alkoxy_{(C≤12)}, acyloxy_{(C≤12)}, aryloxy_{(C≤12)}, heteroaryloxy_{(C≤12)}, heterocycloalkoxy_{(C≤12)}, amido_{(C≤12)}, alkylamino_{(C≤12)}, dialkylamino_{(C≤12)}, or a substituted version of any of these groups; or
a PEG moiety wherein the PEG moiety is of the formula: —(OCH₂CH₂)ₚOR₉;
   wherein:
   p is 1-20; and
   R₉ is hydrogen, alkyl_{(C≤8)}, or substituted alkyl_{(C≤8)}; or
an antitumor antibiotic linked through a cleavable covalent linker;

R₇ is hydrogen,
alkyl_{(C≤8)}, cycloalkyl_{(C≤8)}, alkenyl_{(C≤8)}, cycloalkenyl_{(C≤8)}, alkynyl_{(C≤8)}, alkoxy_{(C≤8)}, or a substituted version of any of these groups, or an amino protecting group;

X₁ and X₄ are each independently selected from: hydrogen, fluoride, alkyl_{(C1-6)}, or substituted alkyl_{(C1-6)}; or

X₂ and X₃ are each independently selected from: a PEG moiety wherein the PEG moiety is of the formula: —CH₂CH₂)ₙOR₈; wherein:
n is 1-20; and
R₈ is hydrogen, alkyl_{(C≤8)}, or substituted alkyl_{(C≤8)};

L₁ and L₂ are each independently absent, a neutral ligand, or an anionic ligand; and

M is a metal ion;

or a pharmaceutically acceptable salt thereof.

The compounds may be further defined as:

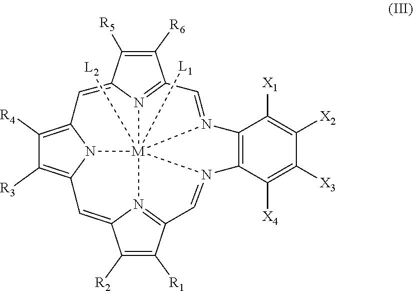

wherein:

R₁-R₆ are each independently selected from: hydrogen, amino, cyano, halo, hydroxy, hydroxyamino, or nitro,
alkyl_{(C≤12)}, cycloalkyl_{(C≤12)}, alkenyl_{(C≤12)}, cycloalkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, alkoxy_{(C≤12)}, acyloxy_{(C≤12)}, aryloxy_{(C≤12)}, heteroaryloxy_{(C≤12)}, heterocycloalkoxy_{(C≤12)}, amido_{(C≤12)}, alkylamino_{(C≤12)}, dialkylamino_{(C≤12)}, or a substituted version of any of these groups; or
an antitumor antibiotic linked through a cleavable covalent linker;

X₁ and X₄ are each independently selected from: hydrogen, fluoride, alkyl_{(C1-6)}, or substituted alkyl_{(C1-6)}; or

X₂ and X₃ are each independently selected from: a PEG moiety wherein the PEG moiety is of the formula: —(OCH₂CH₂)ₙOR₈; wherein:
n is 1-20; and
R₈ is hydrogen, alkyl_{(C≤8)}, or substituted alkyl_{(C≤8)};

L₁ and L₂ are each independently absent, a neutral ligand, or an anionic ligand; and

M is a metal ion;

or a pharmaceutically acceptable salt thereof.

In some embodiments, the compounds are further defined as:

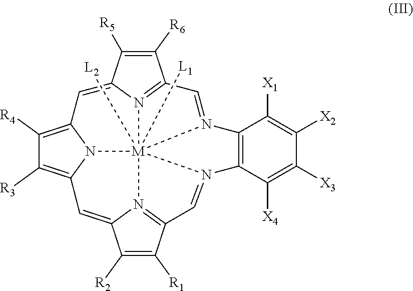

wherein:

R₁, R₂, R₅ and R₆ are each independently selected from hydrogen, alkyl_{(C≤12)}, or substituted alkyl_{(C≤12)}; or
an antitumor antibiotic linked through a cleavable covalent linker;

R₃ and R₄ are each independently selected from hydrogen, alkyl_{(C≤12)}, or substituted alkyl_{(C≤12)};

X₁ and X₄ are each independently selected from: hydrogen, fluoride, alkyl_{(C1-6)}, or substituted alkyl_{(C1-6)}; or

X₂ and X₃ are each independently selected from: a PEG moiety wherein the PEG moiety is of the formula: —(OCH₂CH₂)ₙOR₈; wherein:
n is 1-20; and
R₈ is hydrogen, alkyl_{(C≤8)}, or substituted alkyl_{(C≤8)};

L₁ and L₂ are each independently absent, a neutral ligand, or an anionic ligand; and

M is a metal ion;

or a pharmaceutically acceptable salt thereof.

In some embodiments, the antitumor antibiotic is an anthracycline antibiotic. The antitumor antibiotic may be further defined by the formula:

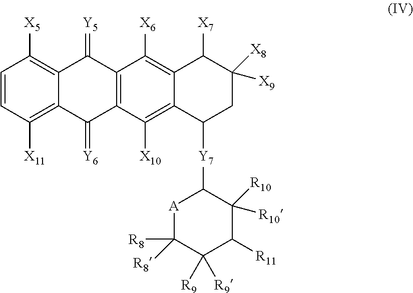

wherein:

X₅, X₆, X₇, X₁₀, and X₁₁ are each independently hydrogen, halo, hydroxy, carboxy, ester_{(C≤12)}, substituted ester_{(C≤12)}, alkoxy_{(C≤12)}, or substituted alkoxy_{(C≤12)};

X₈ is a covalent bond to the linker, acyl_{(C≤18)} or substituted acyl_{(C≤18)};

X₉ is hydrogen, hydroxy, alkoxy_{(C≤12)}, or substituted alkoxy_{(C≤12)};

Y₅, Y₆, and Y₇ are each independently O, S, or NH;

A is O or S; and

R₈, R₈′, R₉, R₉′, R₁₀, R₁₀′, and R₁₁ are each independently hydrogen, amino, halo, hydroxy, mercapto, or
alkyl_{(C≤8)}, alkoxy_{(C≤8)}, alkylthio_{(C≤8)}, alkylamino_{(C≤8)}, dialkylamino_{(C≤8)}, or a substituted version of any of these groups.

The antitumor antibiotic may be further defined by the formula:

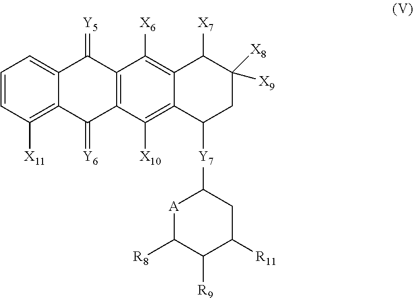

wherein:

X₆, X₇, X₁₀, and X₁₁ are each independently hydrogen, halo, hydroxy, carboxy, ester_{(C≤12)}, substituted ester_{(C≤12)}, alkoxy_{(C≤12)}, or substituted alkoxy_{(C≤12)};

X₈ is a covalent bond to the linker, acyl_{(C≤18)} or substituted acyl_{(C≤18)};

X₉ is hydrogen, hydroxy, alkoxy_{(C≤12)}, or substituted alkoxy_{(C≤12)};

Y₅, Y₆, and Y₇ are each independently O, S, or NH;

A is O or S; and

R₈, R₉, and R₁₁ are each independently hydrogen, amino, halo, hydroxy, mercapto, or alkyl_{(C≤8)}, alkoxy_{(C≤8)}, alkylthio_{(C≤8)}, alkylamino_{(C≤8)}, dialkylamino_{(C≤8)}, or a substituted version of any of these groups.

In some embodiments, the antitumor antibiotic is further defined by the formula:

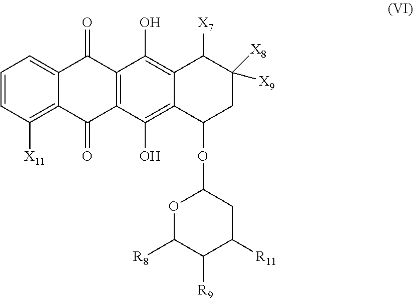

wherein:

X₇ and X₁₁ are each independently hydrogen, halo, hydroxy, carboxy, ester_{(C≤12)}, substituted ester_{(C≤12)}, alkoxy_{(C≤12)}, or substituted alkoxy_{(C≤12)};

X₈ is a covalent bond to the linker, acyl_{(C≤18)} or substituted acyl_{(C≤18)};

X₉ is hydrogen, hydroxy, alkoxy_{(C≤12)}, or substituted alkoxy_{(C≤12)}; and

R₈, R₉, and R₁₁ are each independently hydrogen, amino, halo, hydroxy, mercapto, or alkyl_{(C≤8)}, alkoxy_{(C≤8)}, alkylthio_{(C≤8)}, alkylamino_{(C≤8)}, dialkylamino_{(C≤8)}, or a substituted version of any of these groups.

The antitumor antibiotic may be doxorubicin, daunorubicin, epirubicin, idarubicin, pirarubicin, aclarubicin, or mitoxantrone. In some embodiments, the antitumor antibiotic is doxorubicin or daunorubicin. The antitumor antibiotic may be doxorubicin.

In some embodiments, the antitumor antibiotic is linked to the texaphyrin core through a cleavable covalent linker, wherein the cleavable linker is a disulfide, a ketal, an acetal, a germinal dialcohol, an ester, a carbamate, a carbonate, an oxime, a hydrazone, or a peptide sequence which undergoes enzymatic cleavage. The cleavable covalent linker may be a disulfide, a ketal, an acetal, a germinal dialcohol, an ester, a carbamate, a carbonate, an oxime, or a hydrazone. In some embodiments, the cleavable covalent linker is a disulfide. In other embodiments, the cleavable covalent linker is a hydrazone. The antitumor antibiotic may be linked through a cleavable covalent linker is further defined as:

-Y₅-A₁-Y₆-A₂-Y₇-A₃-

wherein:

Y₅, Y₆, and Y₇ are each independently selected from absent, alkanediyl_{(C≤12)}, alkenediyl_{(C≤12)}, arenediyl_{(C≤12)}, or a substituted version of any of these groups;

A₁ and A₃ are each independently selected from absent, —C(O)O—, —C(O)NH—, —OC(O)O—, —OC(O)NH—, —NHC(O)NH—, —C(NRₐ)O—, —C(NRₐ)NH—, —OC(NRₐ)O—, —OC(NRₐ)NH—, —NHC(NRₐ)NH—; wherein:
Rₐ is hydrogen, alkyl_{(C≤6)}, or substituted alkyl_{(C≤6)}; and

A₂ is a cleavable covalent linker.

In some embodiments, Y₅ is alkanediyl_{(C1-8)} or substituted alkanediyl_{(C1-8)} such as —CH₂CH₂CH₂—. Y₆ may be alkanediyl_{(C1-8)} or substituted alkanediyl_{(C1-8)} such as —CH₂CH₂—. In other embodiments, Y₆ is absent. In some embodiments, Y₇ is alkanediyl_{(C1-8)} or substituted alkanediyl_{(C1-8)} such as —CH₂CH₂—. In other embodiments, Y₇ is absent. A₁ may be —OC(O)O—, —OC(O)NH—, or —NHC(O)NH—, specifically —OC(O)NH—. In some embodiments, A₃ is —OC(O)O—, —OC(O)NH—, or —NHC(O)NH—, specifically —OC(O)NH—.

In some aspects, A₂ is a cleavable covalent linker selected from a disulfide, a ketal, an acetal, a germinal dialcohol, an ester, a carbamate, a carbonate, an oxime, a hydrazone, and a peptide sequence which undergoes enzymatic cleavage. A₂ may be a peptide sequence which undergoes enzymatic cleavage. In other embodiments, A₂ is a disulfide, a ketal, an acetal, a germinal dialcohol, an ester, a carbamate, a carbonate, an oxime, or a hydrazone. In one embodiment, A₂ is a disulfide. In other embodiment, A₂ is a hydrazone.

R₁ and R₆ may both be alkyl_{(C1-6)} or substituted alkyl_{(C1-6)} such as methyl. In some embodiments, R₃ and R₄ are alkyl_{(C1-6)} or substituted alkyl_{(C1-6)} such as ethyl. In some embodiments, X₂ and X₃ are a PEG moiety of the formula: —(OCH₂CH₂)ₙOR₈; wherein:

n is 1-10; and

R₈ is alkyl_{(C≤8)} or substituted alkyl_{(C≤8)}.

In some embodiments, X₂ and X₃ are a PEG moiety of the formula: —(OCH₂CH₂)ₙOR₈; wherein:

n is 1-5; and

R₈ is alkyl_{(C≤8)}.

X₂ and X₃ may be —OCH₂CH₂OCH₂CH₂OCH₂CH₂OCH₃.

In some embodiments, M is a gadolinium atom such as Gd(III). L₁ and L₂ may be anionic ligands. In some embodiments, L₁ and L₂ are acylate_{(C≤12)} or substituted acylate_{(C≤12)} such as acetate.

In some embodiments, the compound is further defined as:

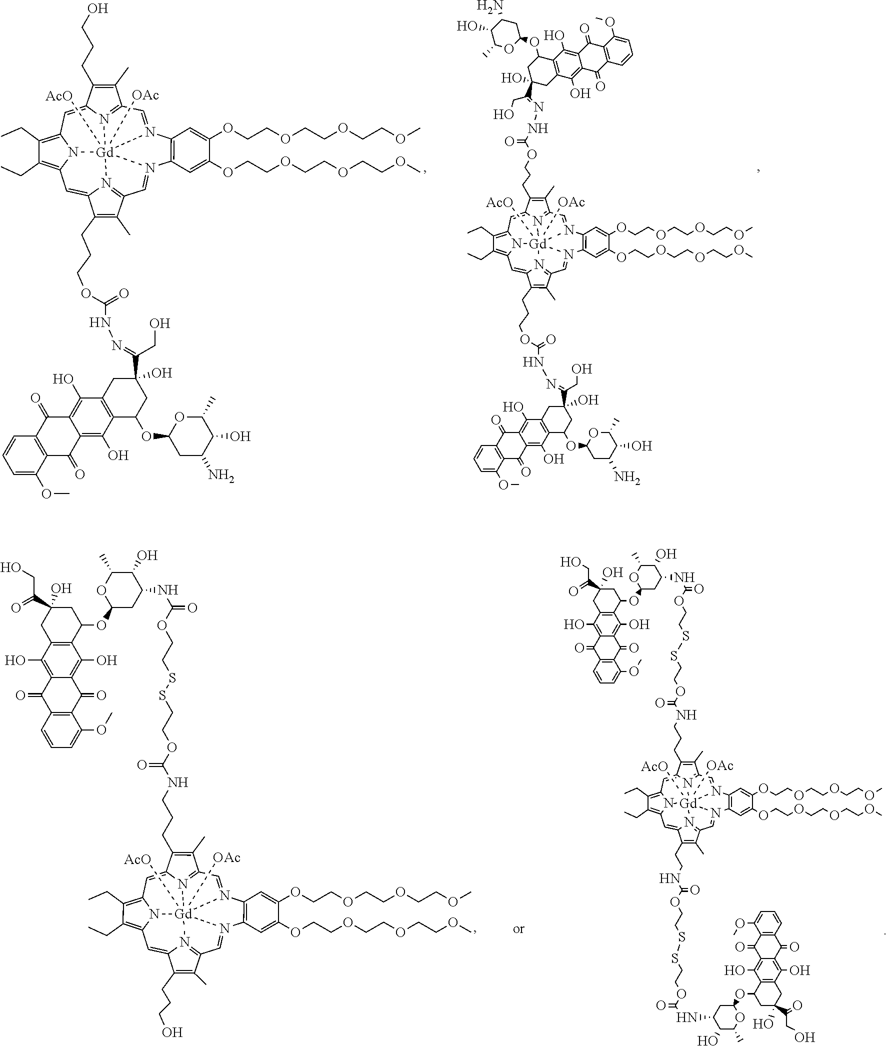

or a pharmaceutically acceptable salt thereof.

In some embodiments, the compound is further defined as:

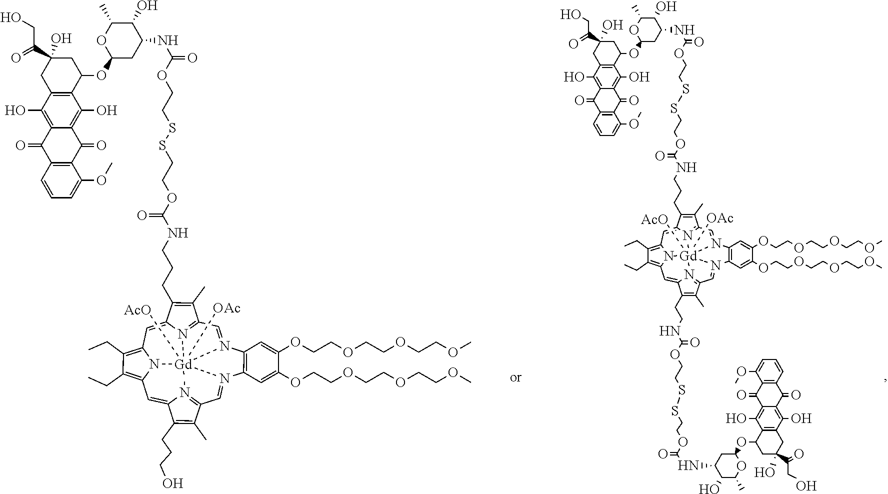

or a pharmaceutically acceptable salt thereof.

In still yet another aspect, the present disclosure provides pharmaceutical composition comprising:

(A) a compound as described herein; and

(B) an excipient.

The pharmaceutical composition may be formulated for oral administration or administration by injection. In one embodiment, the pharmaceutical composition is formulated for administration by injection such as formulated for intraarterial administration, intraperitoneal administration, or intravenous administration. The pharmaceutical composition may be formulated as a liposome. In some embodiments, the pharmaceutical composition is formulated as a unit dose.

In still yet another aspect, the present disclosure provides methods of treating a disease or disorder in a patient in need thereof comprising administering to the patient a therapeutically effective amount of a compound or a composition described herein. In some embodiments, the disease or disorder is cancer. The cancer may be a carcinoma, sarcoma, lymphoma, leukemia, melanoma, mesothelioma, multiple myeloma, or seminoma. The cancer may be a cancer of the bladder, blood, bone, brain, breast, central nervous system, cervix, colon, endometrium, esophagus, gall bladder, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid. In some embodiments, the cancer is leukemia, Hodgkin's lymphoma, bladder cancer, breast cancer, colon cancer, stomach cancer, lung cancer, liver cancer, ovarian cancer, a sarcoma of the soft tissue, or multiple myeloma. The cancer may be colon cancer, liver cancer, or lung cancer. In some embodiments, the methods further comprise administering a second anti-cancer therapy. The second anti-cancer therapy may be another chemotherapeutic drug, surgery, radiotherapy, photodynamic therapy, sonodynamic therapy, cryotherapy, or immunotherapy.

The compound or composition may be administered once. Alternatively, the compound or composition may be administered two or more times.

In still yet another aspect, the present disclosure provides method of imaging a patient comprising:

(A) administering the compound or pharmaceutical composition described herein; and

(B) imaging the patient to determine the presence of a tumor.

In some embodiments, patient is imaged using MRI, CT, SPECT, SPECT/MRI, or SPECT/CT. The tumor may be cancer such as a carcinoma.

It is contemplated that any method or composition described herein can be implemented with respect to any other method or composition described herein.

The terms “comprise” (and any form of comprise, such as “comprises” and “comprising”), “have” (and any form of have, such as “has” and “having”), “contain” (and any form of contain, such as “contains” and “containing”), and “include” (and any form of include, such as “includes” and “including”) are open-ended linking verbs. As a result, a method, composition, kit, or system that “comprises,” “has,” “contains,” or “includes” one or more recited steps or elements possesses those recited steps or elements, but is not limited to possessing only those steps or elements; it may possess (i.e., cover) elements or steps that are not recited. Likewise, an element of a method, composition, kit, or system that “comprises,” “has,” “contains,” or “includes” one or more recited features possesses those features, but is not limited to possessing only those features; it may possess features that are not recited.

Any embodiment of any of the present methods, composition, kit, and systems may consist of or consist essentially of—rather than comprise/include/contain/have—the described steps and/or features. Thus, in any of the claims, the term “consisting of” or “consisting essentially of” may be substituted for any of the open-ended linking verbs recited above, in order to change the scope of a given claim from what it would otherwise be using the open-ended linking verb.

The use of the term “or” in the claims is used to mean “and/or” unless explicitly indicated to refer to alternatives only or the alternatives are mutually exclusive, although the disclosure supports a definition that refers to only alternatives and “and/or.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A & 1B show (FIG. 1A) Schematic illustration of the proposed Dox release and fluorescence enhancement produced by FL-1 upon exposure to cellular thiols. (FIG. 1B) Subcutaneous (S.C.) xenograft and metastatic liver cancer models prepared using the KB and CT26 cell lines, respectively. The proposed accumulation of free Dox in the resulting cancerous lesions after administration of FL-1 via tail-vein injection and conjugate cleavage is also shown, as are the two potential modes of tumor imaging

FIGS. 2A-2D show absorption (FIG. 2A) and fluorescence spectra (FIG. 2B) of FL-1 and FL-10 (5 μM, respectively) recorded in the absence and presence of GSH (5 mM) in PBS buffer (10 mM, pH 7.4). (FIG. 2C) Fluorescence spectra of FL-1 (5 μM) recorded in the presence of different concentrations of GSH in PBS buffer (10 mM, pH 7.4). (FIG. 2D) Fluorescence intensity (FI) at 592 nm determined in the absence and presence of GSH (5 mM) at different pH values. All measurements were made at 37° C. using an excitation wavelength of 500 nm.

FIGS. 3A & 3B show (FIG. 3A) Absorption and (FIG. 3B) fluorescence spectra of 1 (5 μM) recorded in the absence and presence of GSH (5 mM). All data were obtained using PBS buffer (10 mM, pH 7.4) at 37° C. with an excitation wavelength at 500 nm.

FIGS. 4A & 4B show (FIG. 4A) Excitation spectrum of 1 and (FIG. 4B) normalized absorption spectrum of Dox.

FIGS. 5A & 5B show (FIG. 5A) Fluorescence spectral changes of 1 (5 μM) as a function of time as seen in the presence of GSH (5 mM). (FIG. 5B) Fluorescence intensity (FI) at 592 nm of 1 recorded as a function of time in the absence and presence of GSH (5 mM), respectively. All data were recorded in PBS buffer (10 mM, pH 7.4) at 37° C. with an excitation wavelength at 500 nm.

FIGS. 6A & 6B show (FIG. 6A) Fluorescence spectra and (FIG. 6B) fluorescence intensity (FI) at 592 nm of 1 (5 μM) recorded in the presence of different concentrations of GSH. All experiments were carried out in PBS buffer (10 mM, pH 7.4) at 37° C. using an excitation wavelength at 500 nm.

FIG. 7 shows the fluorescence response of 1 (5 μM) with and without GSH (5 mM) as observed at different pH values. The histogram shows the florescence intensity (FI) at 592 nm of 1 recorded in the absence (white bar) and presence (black bar) of GSH (5 mM), respectively. All experiments were carried out at 37° C. using an excitation wavelength of 500 nm.

FIG. 8 shows the HPLC chromatograms of conjugate 1, C, Dox, and 1 in the presence of GSH (5 mM) in PBS buffer of pH 7.4 at 37° C. Peaks in the chromatograms were detected by monitoring the UV/Vis absorption at 500 nm. All peaks were identified by UV/Vis absorption and ESI-MS spectroscopic analysis.

FIGS. 9A & 9B show (FIG. 9A) Fluorescence response of FL-1 (5 μM) with and without GSH (5 mM). Excitation was effected at 500 nm. (FIG. 9B) Dox released from FL-1 (5 μM) as a function of time in the presence and absence of GSH (5 mM). Dox in HPLC chromatograms was detected by UV/Vis absorption using 500 nm as the interrogation wavelength. All data were recorded in PBS buffer (10 mM, pH 7.4) at 37° C.

FIGS. 10A & 10B show (FIG. 10A) Fluorescence response of 1 (20 μM) with and without GSH (5 mM). Excitation was effected at 500 nm. (FIG. 10B) Dox released from 1 (20 μM) as a function of time in the presence and absence of GSH (5 mM). Dox in HPLC chromatograms was detected by UV/Vis absorption using 500 nm as the interrogation wavelength. All data were recorded in PBS buffer (10 mM, pH 7.4) at 37° C.

FIG. 11 shows fluorescence images of FL-1-treated cells. Folate receptor positive (KB, CT26) and negative (HepG2, NIH3T3) cells lines were treated with 4 μM of FL-1 for 1 h. The cells were then fixed in 4% paraformaldehyde after washing with PBS and staining with

Hoechst (nuclear counterstain, blue). Scale bar: 20 μm.

FIG. 12A-D shows the quantitative analysis of cellular uptake of FL-1 into the KB (FIG. 12A), CT26 (FIG. 12B), HepG2 (FIG. 12C), and NIH3T3 (FIG. 12D) cell lines as inferred from flow cytometry.

FIG. 13 shows the fluorescence images of cells treated with conjugates FL-1 and L-1. The KB and CT26 cells were treated with 4 μM of each formulation for 1 h. The cells were then fixed in 4% paraformaldehyde after washing with PBS, and then stained with Hoechst (nuclear counterstain, blue). Scale bar: 20 μm.

FIG. 14 show anti-proliferative activity of FL-1 in various cell lines as inferred from

MTT assays. Folate receptor positive (KB, CT26) and negative (HepG2, NIH3T3) cell lines were treated with various concentration of FL-1 for 48 h prior to analysis.

FIGS. 15A & 15B show the comparison of anti-proliferative effect of FL-1 and FL-10 in KB (FIG. 15A) and CT26 (FIG. 15B) cell lines. Cells were treated with various concentration of FL-1 or FL-10 for 48 h, respectively, and then analyzed via the MTT assay described above.

FIGS. 16A & 16B show (FIG. 16A) T₁ relaxivity measurements of FL-1 in PBS solution as a function of concentrations at 60 and 200 MHz. The relaxivities were calculated to be 11.8±0.3 and 7.1±0.4 mM⁻¹s⁻¹ at 60 and 200 MHz, respectively. (FIG. 16B) T₁-weighted spin-echo MR phantom images recorded at different concentrations of FL-1.

FIGS. 17A & 17B show fluorescence (FIG. 17A) and T₁-weighted MR (FIG. 17B) images of KB cell pellets obtained from cells treated with various concentrations of FL-1 for 12 h.

FIGS. 18A-18D show (FIG. 18A) whole-body in vivo fluorescence images recorded 6 h after intravenous injection of FL-1 to nude mice bearing KB cell-derived tumors (S.C.

xenograft model). (FIG. 18B) Fluorescence microscopy images of cryo-sectioned tumor tissues taken from the S.C. xenograft animals 24 h after FL-1 administration. (FIG. 18C) Signal-to-noise ratio (SNR) for MR images of the tumor tissue for this same model. (FIG. 18D) Tumor volume vs. time for S.C. xenograft mice treated with saline and FL-1 (n=5).

FIGS. 19A & 19B show (FIG. 19A) T₁-weighted MR images of FL-1 for early diagnosis in metastatic liver cancer mice. Yellow arrow indicates tumor area. (FIG. 19B) Comparison of signal-to-noise ratio (SNR) between normal and tumor region in liver tissue.

FIGS. 20A & 20B show (FIG. 20A) T2-weighted MR images showing the livers of nude mice recorded at the indicated times post-inoculation with CT26 cells (metastatic liver cancer model). Red circles indicated the metastatic tumors (FIG. 20B) Kaplan-Meier curves showing the cumulative survival rates of metastatic liver model mice after injection with either saline or FL-1. Survival was enhanced for FL-1 relative to saline control.

FIGS. 21A & 21B show the HPLC trace (FIG. 21A) and the high resolution ESI mass spectrum (FIG. 21B) of conjugate 1.

FIGS. 22A-22D show (FIG. 22A) Absorption and (FIG. 22B) fluorescence spectra of conjugate 11 and Dox recorded in PBS buffer (pH 7.4). (FIG. 22C) Time-dependent fluorescence spectral changes seen for a solution of conjugate 11 (10 μM) in acetate buffer (pH 5.0). (FIG. 22D) FI (fluorescence intensity) at 593 nm recorded as a function of time in PBS (pH 7.4) and acetate buffer (pH 5.0) containing 1% (v/v) of DMSO in both cases. All studies were carried out at 37° C. Fluorescence data were recorded using an excitation wavelength of 500 nm.

FIG. 23 shows the normalized fluorescence spectrum of conjugate 11 in PBS buffer (pH 7.4) at 37° C. with an excitation wavelength at 500 nm.

FIG. 24 shows the HPLC chromatograms of conjugate 11 at an acidic pH (acetate buffer; pH 5.0), doxorubicin, and 2. Peaks in the chromatograms were detected by monitoring the UV/Vis absorption at 500 (pink) and 470 nm (black), respectively.

FIG. 25 shows the ESI-Mass spectrum for the Dox released from conjugate 11 when allowed to sit in an acetate buffer at pH 5.0.

FIG. 26 shows fluorescence images of cells treated with conjugate 11. In these studies, A549, CT26, and NIH3T3 cells were treated with 4 μM of 11 for 1 h. The cells were then fixed in 4% paraformaldehyde after washing with PBS and then stained with Hoechst (nuclear counterstain, blue). Images were obtained using excitation wavelengths of 405 nm and 543 nm, with the emission being monitored over the 420-480 nm and 560-615 nm spectral regions for the blue and red signals, respectively. Scale bar: 20 μm.

FIG. 27 shows fluorescence images of cells treated with conjugate 11. CT26 and NIH3T3 cells were treated with 4 μM of 11 for 12 h. The cells were then fixed in 4% paraformaldehyde after washing with PBS and then stained with Hoechst (nuclear counterstain, blue). Images were obtained using excitation wavelengths of 405 nm and 543 nm, with the emission being monitored over the 420-480 nm and 560-615 nm spectral regions for the blue and red signals, respectively. Scale bar: 20 μm.

FIGS. 28A-28F show the fluorescence images of CT26 cells treated with Hoechst (blue) (FIG. 28A), LysoTracker (green) (FIG. 28B), and conjugate 11 (red) (FIG. 28C). Cells were incubated with 10 μM of conjugate 11 for 12 h. The cells were then fixed in 4% paraformaldehyde after washing in PBS and stained with Hoechst and LysoTracker. All images were merged in panel (FIG. 28D) and a partial image was magnified in panel (FIG. 28E). The white arrows in the magnified image show the co-localization of conjugate 11 with the lysosome-selective dye (LysoTracker). Excitation was at 405 nm, 480 nm, and 543 nm; the emission was monitored over the 420-480 nm, 505-550 nm, and 560-615 nm spectral regions for the blue, green, and red signals, respectively. Scale bar: 10 μm. (FIG. 28F) Quantification of the relative co-localization of the conjugate 11 within lysosomes and mitochondria in CT26 cells based on Pearson's correlation coefficient. At least 7 cells were measured in 2 different regions in each experiment. ** denotes P<0.01 by Student's t test.

FIG. 29 shows fluorescence images of CT26 cells treated with Hoechst (blue), MitoTracker (green), and conjugate 11 (red). Cells were treated with 10 μM of conjugate 11 for 12 h. Cells were then fixed in 4% paraformaldehyde after washing in PBS and stained with Hoechst (blue) and MitoTracker (green). Scale bar: 10 μm.

FIG. 30 shows the antiproliferative activity of conjugate 11 in various cell lines. Cells were incubated with various concentrations of 11 for 48 h before being analyzed using a standard MTT assay.

FIGS. 31A-31C shows (FIG. 31A) Concentration dependent T₁ relaxivity studies of conjugate 11 in PBS solution at 60 and 200 MHz. The T₁ relaxivities were 20.1±0.4 and 6.1±0.2 mM⁻¹s⁻¹ at 60 and 200 MHz, respectively. (FIG. 31B) T₁-weighted spin-echo MR phantom image determined in PBS at 200 MHz. (FIG. 31C) T₁-weighted MR images of cell pellets of A549 and CT26 cells incubated with different concentrations of conjugate 11 at 200 MHz.

FIGS. 32A & 32B show the HPLC trace (FIG. 32A) and the high resolution ESI mass spectrum (FIG. 32B) of conjugate 11.

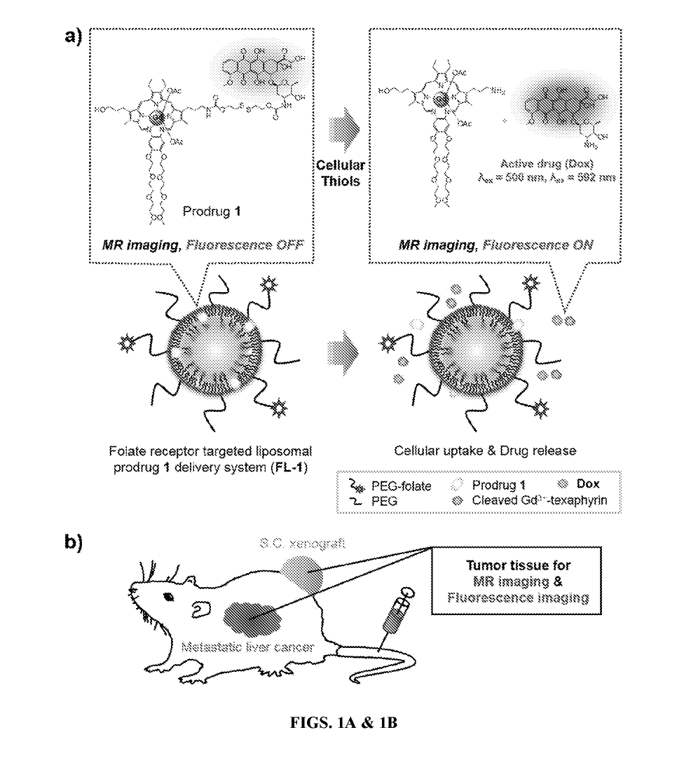

### DESCRIPTION OF ILLUSTRATIVE EMBODIMENTS

The present disclosure describes conjugates with a texaphyrin compound and a second anticancer compound such as an antitumor antibiotics. One non-limiting example of the second anticancer compound include antitumor antibiotics such as anthracycline antibiotics. The conjugates described herein may include a metal chelated texaphyrin joined to an anthracycline antibiotic such as doxorubicin or daunomycin. These conjugates may be used to increase the effectiveness of the antitumor antibiotics, delivery of the antitumor antibiotic to the target cells, or allow for the monitoring of the delivery of the therapeutic agent. In particular, the selective delivery of these antitumor antibiotics such that the antibiotic may be primarily released in tumor cells are contemplated.

### A. Antitumor Antibiotics

In some aspects, the present disclosure provides compounds wherein an antitumor antibiotic is linked to a texaphyrin compound. Antitumor antibiotics are a group of chemotherapeutic agents which are useful for the treatment of a hyperproliferative diseases such as cancer by damaging the DNA of the diseased cells. Two major classes of antitumor antibiotics include anthracycline antitumor antibiotics, chromomycin derivatives such as dactinomycin and plicamycin, and other antibacterial compounds such as mitomycin and bleomycin.

In some embodiments, the present disclosure relates to using anthracycline antitumor antibiotics such as doxorubicin and daunorubicin which share a common polyketide core which is usually reddish in color with one or more linked sugar or sugar derivative residues. Many of the natural products from which this class of compounds was developed are produced as byproducts from Streptomyces bacteria especially the bacteria Streptomyces peuetius var. caesius. The common polyketide core contains 3, 4, or 5 rings in which at least 2 of the rings are aromatic. The common core may further contain one or more carbonyl groups, hydroxy groups, or C1-C6 alkoxy or acyloxy groups. In particular, the common polyketide core may be further defined as:

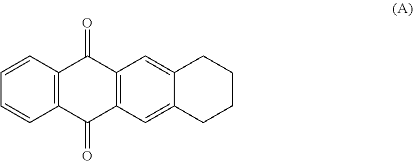 [SMILES: O=C1c2ccccc2C(=O)c2cc3c(cc21)CCCC3]

In some embodiments, the anthracycline antitumor antibiotic comprises one or more sugar or sugar derivative residues which have been covalently linked to the polyketide core. These sugar or sugar derivative residues contain one or more amino groups in addition to hydroxy groups, C1-C6 alkoxy groups, C1-C6 acyloxy groups, or C1-C6 alkyl groups. Some non-limiting examples of anthracycline antitumor antibiotic include daunorubicin, doxorubicin, epirubicin, or idarubicin. Additional examples of anthracycline antitumor antibiotics include those described in Rabbani, et al., 2005; Kizek, et al., 2012; Olano, et al., 2009; Cera and Palumbo, 1990; and Fritzsche, et al., 1987. In some aspects, the present composition may comprise a daunorubicin or doxorubicin linked to a texaphyrin compound.

### B. Texaphyrin Compounds

Texaphyrin compounds are a pentadentate macrocyclic compound often characterized as an “expanded porphyrin” with three pyrrole rings and two nitrogen atoms from two Schiff bases. These compound and the corresponding metal complexes have been shown to be useful as MRI contrast agents, photodynamic therapy agents, and radiosensitizers. Texaphyrin compounds are known to exist in two forms: an sp² form and a sp³ form. The fully aromatized sp² form is more stable and the form that traditionally exists in metal complexes. The sp³ form readily undergoes oxidation and thus is more generally more difficult to isolate. During metallation, the texaphyrin compound undergoes oxidation to forming an extremely tightly bound metal complex which is resistant to removal of the metal ion. This phenomenon is described in U.S. Pat. No. 5,504,205, Shimanovich, et al., 2001 and Hannah, et al., 2001, all of which are incorporated herein by reference. The expanded pentadentate macrocycle is known to bind a wide array of different metal ions including trivalent rare earth ions such as gadolinium and lutetium. One example of a texaphyrin compound described herein is the motexafin core. Non-limiting examples of texaphyrins are taught by U.S. Pat. Nos. 4,935,498, 5,252,270, 5,272,142, 5,292,414, 5,369,101, 5,432,171, 5,439,570, 5,504,205, 5,569,759, 5,583,220, 5,587,463, 5,591,422, 5,633,354, 5,776,925, 5,955,586, 5,994,535, 6,207,660, 7,112,671, and 8,410,263, which are all incorporated herein by reference.

The texaphyrin compounds described herein are shown, for example, above, in the summary section and in the claims below. These texaphyrin compounds may be made using the synthetic methods outlined in the Examples section or as described U.S. Pat. Nos. 4,935,498, 5,252,270, 5,272,142, 5,292,414, 5,369,101, 5,432,171, 5,439,570, 5,504,205, 5,569,759, 5,583,220, 5,587,463, 5,591,422, 5,633,354, 5,776,925, 5,955,586, 5,994,535, 6,207,660, 7,112,671, and 8,410,263, which are all incorporated herein by reference. These methods can be further modified and optimized using the principles and techniques of organic chemistry as applied by a person skilled in the art. Such principles and techniques are taught, for example, in Smith, March's Advanced Organic Chemistry: Reactions, Mechanisms, and Structure, (2013), which is incorporated by reference herein. In addition, the synthetic methods may be further modified and optimized for preparative, pilot- or large-scale production, either batch or continuous, using the principles and techniques of process chemistry as applied by a person skilled in the art. Such principles and techniques are taught, for example, in Anderson, Practical Process Research & Development—A Guide for Organic Chemists (2012), which is incorporated by reference herein.

### C. Cleavable Linker Groups

In some aspects, the present disclosure provides compositions of two components, namely a texaphyrin compound and an antitumor antibiotic, which are joined with a linker that is cleavable in vivo. The linker may include covalent groups such as a hyrazone, a disulfide, an ester, a carbonate, a carbamate, a ketal, an acetal, a germinal dialcohol, or an oxime. Additionally, the cleavable linker may be a polypeptide group which is cleavable by an enzyme present in the target sale. In some embodiments, the cleavable linker group is an acid sensitive group which undergoes hydrolysis in vivo. Acid sensitive groups may be used in the compounds described herein and especially for use in cancer cells due to the acidic nature of most cancer cells.

In other embodiments, the cleavable linker group is a group such as a disulfide which undergoes exchange in vivo. These cleavable linker groups undergo exchange with free thiols in the cell such that the antitumor antibiotic is released specifically within the cell. In addition to the specific cleavage method, the other components may facilitate the selective uptake of the composition into certain types of cells. For example, the texaphyrin compound may show increased uptake within cancer cells thus leading to increased concentration of the composition into the cancer cells.

In another aspect, the present compounds may contain a texaphyrin compound and an antitumor antibiotic joined by a linker which forms a covalent bond that is reversible in vivo. Reversible covalent bonds, such as a hydrazone, a ketal, or an acetal, are readily hydrolyzed in acidic conditions in vivo such as tumor cells. Some of the conjugates (or compounds) described herein may be joined by a linker which contains a cleavable hydrazone linker. Other embodiments of the present disclosure relate to compounds which do not contain a hydrazone linker.

Finally, the two components of the conjugates described herein may be linked with a peptide linker which is cleaved by a protease in vivo. Some non-limiting examples of a protease include serine protease, cysteine protease, threonine protease, aspartic protease, glutamic protease, metalloprotease, or lyase. These proteases may be either an endoprotease or an exoprotease. In some embodiments, the peptide linker may contain other groups which generate a free antitumor antibiotic, texaphyrin compound, or both.

D. Compound Characteristics

All of the texaphryin conjugates of the present disclosure may be useful for the prevention and treatment of one or more diseases or disorders discussed herein or otherwise. In some embodiments, one or more of the compounds characterized or exemplified herein as an intermediate, a metabolite, and/or prodrug, may nevertheless also be useful for the prevention and treatment of one or more diseases or disorders. As such unless explicitly stated to the contrary, all of the compounds of the present disclosure are deemed “active compounds” and “therapeutic compounds” that are contemplated for use as active pharmaceutical ingredients (APIs). Actual suitability for human or veterinary use is typically determined using a combination of clinical trial protocols and regulatory procedures, such as those administered by the Food and Drug Administration (FDA). In the United States, the FDA is responsible for protecting the public health by assuring the safety, effectiveness, quality, and security of human and veterinary drugs, vaccines and other biological products, and medical devices.

In some embodiments, the texaphyrin conjugates of the present disclosure have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

The texaphyrin conjugates of the present disclosure may contain one or more asymmetrically-substituted carbon or nitrogen atoms, and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. The compounds may occur as racemates and racemic mixtures, single enantiomers, diastereomeric mixtures and individual diastereomers. In some embodiments, a single diastereomer is obtained. The chiral centers of the compounds of the present disclosure can have the S or the R configuration.

Chemical formulas used to represent the texaphyrin conjugates of the present disclosure will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

In addition, atoms making up the texaphyrin conjugates of the present disclosure are intended to include all isotopic forms of such atoms except where specially noted. Isotopes, as used herein, include those atoms having the same atomic number but different mass numbers. By way of general example and without limitation, isotopes of hydrogen include tritium and deuterium, isotopes of carbon include ¹³C and ¹⁴C, isotopes of oxygen include ¹⁷O and ¹⁸O, and isotopes of nitrogen include ¹⁵N. Additionally, the metal ions in the present invention can have different oxidation states unless otherwise noted. As used herein, the charge on the metal atom can be denoted either as a superscript such as Gd^{III} or using parenthesis such as Gd(III). These two forms are identical as would be recognized to a person of skill in the art. Even if one form is used in the application to describe the oxidation state in one place in the application, it is contemplated that the other form could be used in elsewhere in the application.

The texaphyrin conjugates of the present disclosure may also exist in prodrug form. Since prodrugs are known to enhance numerous desirable qualities of pharmaceuticals (e.g., solubility, bioavailability, manufacturing, etc.), the compounds employed in some methods of the invention may, if desired, be delivered in prodrug form. Thus, the invention contemplates prodrugs of compounds of the present invention as well as methods of delivering prodrugs. Prodrugs of the compounds employed in the invention may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a subject, cleaves to form a hydroxy, amino, or carboxylic acid, respectively.

It should be recognized that the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

It will appreciated that many organic compounds can form complexes with solvents in which they are reacted or from which they are precipitated or crystallized. These complexes are known as “solvates.” Where the solvent is water, the complex is known as a “hydrate.” It will also be appreciated that many organic compounds can exist in more than one solid form, including crystalline and amorphous forms. All solid forms of the compounds provided herein, including any solvates thereof are within the scope of the present disclosure.

### E. Indications

The texaphyrin and antitumor antibiotic conjugates described herein may be used in a variety of different indications such as a hyperproliferative disease.

A. Hyperproliferative Diseases

In some aspects, the texaphyrin and antitumor antibiotic conjugates of the present disclosure may be used to treat or prevent a hyperproliferative disease, such as cancer. While hyperproliferative diseases can be associated with any medical disorder that causes a cell to begin to reproduce uncontrollably, the prototypical example is cancer. One element of cancer is that the normal apoptotic cycle of the cell is interrupted and thus agents that lead to apoptosis of the cell are important therapeutic agents for treating these diseases. As such, the texaphyrin compounds and compositions described in this disclosure may be effective in treating a variety of different cancer types.

Cancer cells that may be treated with the texaphyrin compounds according to the present disclosure include but are not limited to cells from the bladder, blood, bone, bone marrow, brain, breast, colon, esophagus, gastrointestine, gum, head, kidney, liver, lung, nasopharynx, neck, ovary, prostate, skin, stomach, pancreas, testis, tongue, cervix, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant;

paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In certain aspects, the tumor may comprise an osteosarcoma, angiosarcoma, rhabdosarcoma, leiomyosarcoma, Ewing sarcoma, glioblastoma, neuroblastoma, or leukemia.

### F. Pharmaceutical Formulations and Routes of Administration

For the purpose of administration to a patient in need of such treatment, pharmaceutical formulations (also referred to as a pharmaceutical preparations, pharmaceutical compositions, pharmaceutical products, medicinal products, medicines, medications, or medicaments) comprise a therapeutically effective amount of a compound of the present disclosure formulated with one or more excipients and/or drug carriers appropriate to the indicated route of administration.

In some embodiments, the compounds of the present disclosure are formulated in a manner amenable for the treatment of human and/or veterinary patients. In some embodiments, formulation comprises admixing or combining one or more of the compounds of the present disclosure with one or more of the following excipients: lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol. In some embodiments, e.g., for oral administration, the pharmaceutical formulation may be tableted or encapsulated. In some embodiments, the compounds may be dissolved or slurried in water, polyethylene glycol, propylene glycol, ethanol, corn oil, cottonseed oil, peanut oil, sesame oil, benzyl alcohol, sodium chloride, and/or various buffers. Pharmaceutical formulations may be subjected to conventional pharmaceutical operations, such as sterilization and/or may contain drug carriers and/or excipients such as preservatives, stabilizers, wetting agents, emulsifiers, encapsulating agents such as lipids, dendrimers, polymers, proteins such as albumin, or nucleic acids, and buffers, etc.

Pharmaceutical formulations may be administered by a variety of methods, e.g., orally or by injection (e.g. subcutaneous, intravenous, intraperitoneal, etc.). Depending on the route of administration, the compounds of the present invention may be coated in a material to protect the compound from the action of acids and other natural conditions which may inactivate the compound. To administer the active compound by other than parenteral administration, it may be necessary to coat the compound with, or co-administer the compound with, a material to prevent its inactivation. For example, the active compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes. Additionally, Trapasol®, Travasol®, cyclodextrin, and other drug carrier molecules may also be used in combination with the texaphyrin compounds of the present disclosure. It is contemplated that the compounds of the present disclosure may be formulated with a cyclodextrin as a drug carrier using an organic solvent such as dimethylaceamide with a polyethylene glycol and a poloxamer composition in an aqueous sugar solution. In some embodiments, the organic solvent is dimethylsulfoxide, dimethylformamide, dimethylacetamide, or other biologically compatible organic solvents. Additionally, the composition may be diluted with a polyethylene glycol polymer such as PEG100, PEG200, PEG250, PEG400, PEG500, PEG600, PEG750, PEG800, PEG900, PEG1000, PEG2000, PEG2500, PEG3000, or PEG4000. Additionally, the composition may further comprise one or more poloxamer composition wherein the poloxamer contains two hydrophilic polyoxyethylene groups and a hydrophobic polyoxypropylene or a substituted version of these groups. This mixture may be further diluted using an aqueous sugar solution such as 5% aqueous dextrose solution.

The texaphyrin compounds of the present disclosure may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. The carrier can be a solvent or dispersion medium containing, for example, water, ethanol, polyol (such as, glycerol, propylene glycol, and liquid polyethylene glycol, esters thereof, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin. Additionally, pharmaceutical compositions may be formulated with one or more pH adjusting agents such as a weak acid such as acetic acid, citric acid, phosphoric acid, aspartic acid, glutamic acid, gluconic acid, or lactic acid or a weak base such as ammonia or other amine base.

The texaphyrin compounds of the present disclosure can be administered orally, for example, with an inert diluent or an assimilable edible carrier. The compounds and other ingredients may also be enclosed in a hard or soft shell gelatin capsule, compressed into tablets, or incorporated directly into the subject's diet. For oral therapeutic administration, the compounds of the present invention may be incorporated with excipients and used in the form of ingestible tablets, buccal tablets, troches, capsules, elixirs, suspensions, syrups, wafers, and the like. In some embodiments, the oral formulation may be prepared as a pro-drug including but not limited to medoxomil, acetyl, or other esters. The percentage of the therapeutic compound in the compositions and preparations may, of course, be varied. The amount of the therapeutic compound in such pharmaceutical formulations is such that a suitable dosage will be obtained.

In some embodiments, the therapeutic compound may also be administered topically to the skin, eye, or mucosa. Alternatively, if local delivery to the lungs is desired the therapeutic compound may be administered by inhalation in a dry-powder or aerosol formulation.

In some embodiments, it may be advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical carrier. In some embodiments, the specification for the dosage unit forms of the disclosure are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient. In some embodiments, active compounds are administered at a therapeutically effective dosage sufficient to treat a condition associated with a condition in a patient. For example, the efficacy of a compound can be evaluated in an animal model system that may be predictive of efficacy in treating the disease in a human or another animal.

In some embodiments, the effective dose range for the therapeutic compound can be extrapolated from effective doses determined in animal studies for a variety of different animals. In general a human equivalent dose (HED) in mg/kg can be calculated in accordance with the following formula (see, e.g., Reagan-Shaw et al., 2008, which is incorporated herein by reference):

HED(mg/kg)=Animal dose (mg/kg)×(Animal Kₘ/Human Kₘ)

Use of the Kₘ factors in conversion results in more accurate HED values, which are based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are peculiar to each individual. Nonetheless, a calculated HED dose provides a general guide. Other factors affecting the dose include the physical and clinical state of the patient, the route of administration, the intended goal of treatment and the potency, stability and toxicity of the particular therapeutic formulation.

The actual dosage amount of a compound of the present disclosure or composition comprising a compound of the present disclosure administered to a subject may be determined by physical and physiological factors such as type of animal treated, age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

In some embodiments, the therapeutically effective amount typically will vary from about 0.001 mg/kg to about 1000 mg/kg, from about 0.01 mg/kg to about 750 mg/kg, from about 100 mg/kg to about 500 mg/kg, from about 1 mg/kg to about 250 mg/kg, from about 10 mg/kg to about 150 mg/kg in one or more dose administrations daily, for one or several days (depending of course of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some particular embodiments, the amount is less than 10,000 mg per day with a range of 750 mg to 9,000 mg per day.

In some embodiments, the amount of the active compound in the pharmaceutical formulation is from about 2 to about 75 weight percent. In some of these embodiments, the amount if from about 25 to about 60 weight percent.

Single or multiple doses of the agents are contemplated. Desired time intervals for delivery of multiple doses can be determined by one of ordinary skill in the art employing no more than routine experimentation. As an example, subjects may be administered two doses daily at approximately 12 hour intervals. In some embodiments, the agent is administered once a day.

The agent(s) may be administered on a routine schedule. As used herein a routine schedule refers to a predetermined designated period of time. The routine schedule may encompass periods of time which are identical or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the invention provides that the agent(s) may taken orally and that the timing of which is or is not dependent upon food intake. Thus, for example, the agent can be taken every morning and/or every evening, regardless of when the subject has eaten or will eat.

### G. Combination Therapy

Effective combination therapy may be achieved with a single composition or pharmacological formulation that includes both agents, or with two distinct compositions or formulations, administered at the same time, wherein one composition includes a texaphyrin compound described herein, and the other includes the second agent(s). The other therapeutic modality may be administered before, concurrently with, or following administration of the texaphyrin compound described herein. The therapy using the texaphyrin compound described herein may precede or follow administration of the other agent(s) by intervals ranging from minutes to weeks. In embodiments where the other agent and the compounds or compositions of the present disclosure are administered separately, one would generally ensure that a significant period of time did not expire between the time of each delivery, such that each agent would still be able to exert an advantageously combined effect. In such instances, it is contemplated that one would typically administer the texaphyrin compound described herein and the other therapeutic agent within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of a texaphyrin compound described herein, or the other agent will be desired. In this regard, various combinations may be employed. By way of illustration, where the compounds of the present disclosure are “A” and the other agent is “B”, the following permutations based on 3 and 4 total administrations are exemplary:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are likewise contemplated.

A. Hyperproliferative Diseases

Non-limiting examples of pharmacological agents that may be used in the present invention include any pharmacological agent known to be of benefit in the treatment of a cancer or hyperproliferative disorder or disease. In some embodiments, combinations of the texaphyrin compound described herein with a cancer targeting immunotherapy, radiotherapy, chemotherapy, or surgery are contemplated. Also contemplated is a combination of the texaphyrin compound described herein with more than one of the above mentioned methods including more than one type of a specific therapy. In some embodiments, it is contemplated that the immunotherapy is a monoclonal antibody which targets HER2/neu such trastuzumab (Herceptin®), alemtuzumab (Campath®), bevacizumab (Avastin®), cetuximab (Eribitux®), and panitumumab (Vectibix®) or conjugated antibodies such as ibritumomab tiuxetan (Zevalin®), tositumomab (Bexxar®), brentuximab vedotin (Adcetris®), ado-trastuzumab emtansine (Kadcyla™), or denileukin dititox (Ontak®) as well as immune cell targeting antibodies such as ipilimumab (Yervoy®), tremelimumab, anti-PD-1, anti-4-1-BB, anti-GITR, anti-TIM3, anti-LAG-3, anti-TIGIT, anti-CTLA-4, or anti-LIGHT. Furthermore, in some embodiments, the isotopically enriched texaphyrin compound described herein are envisioned to be used in combination therapies with dendritic cell-based immunotherapies such as Sipuleucel-T (Provenge®) or adoptive T-cell immunotherapies.

Furthermore, it is contemplated that the methods described herein may be used in combination with a chemotherapeutic agent such as PR-171 (Kyprolis®), bortezomib (Velcade®), anthracyclines, taxanes, methotrexate, mitoxantrone, estramustine, doxorubicin, etoposide, vinblastine, vinorelbine, 5-fluorouracil, cisplatin, carboplatin, oxaliplatin, Pt(IV) complexes, topotecan, ifosfamide, cyclophosphamide, epirubicin, gemcitabine, vinorelbine, irinotecan, etoposide, vinblastine, pemetrexed, melphalan, capecitabine, BRAF inhibitors, and TGF-β inhibitors. In some embodiments, the combination therapy is designed to target a cancer such as those listed above.

In some aspects, it is contemplated that the texaphyrin compound described herein may be used in conjunction with radiation therapy. Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly.

Radiation therapy used according to the present disclosure may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors induce a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

Additionally, it is contemplated a texaphyrin compound described herein are used in combination with sonodynamic therapy. The use of texaphyrins in sonodynamic therapy is described in U.S. Pat. No. 6,207,660 incorporated herein by reference. A texaphyrin compound described herein is administered before administration of the sonodynamic agent. The conjugate or composition may be administered as a single dose, or it may be administered as two or more doses separated by an interval of time. Parenteral administration is typical, including by intravenous and interarterial injection. Other common routes of administration can also be employed.

Ultrasound is generated by a focused array transducer driven by a power amplifier. The transducer can vary in diameter and spherical curvature to allow for variation of the focus of the ultrasonic output. Commercially available therapeutic ultrasound devices may be employed in the practice of the invention. The duration and wave frequency, including the type of wave employed may vary, and the preferred duration of treatment will vary from case to case within the judgment of the treating physician. Both progressive wave mode patterns and standing wave patterns have been successful in producing cavitation of diseased tissue. When using progressive waves, the second harmonic can advantageously be superimposed onto the fundamental wave.

One non-limiting sonodynamic agent employed in the present disclosure is ultrasound, particularly is low intensity, non-thermal ultrasound, i.e., ultrasound generated within the wavelengths of about 0.1 MHz and 5.0 MHz and at intensities between about 3.0 and 5.0 W/cm².

Furthermore, it is contemplated that the compounds of the present disclosure can be used in combination with photodynamic therapy: By way of example, a lutetium texaphyrin is administered in solution containing 2 mg/mL optionally in 5% mannitol, USP. Dosages of about 1.0 or 2.0 mg/kg to about 4.0 or 5.0 mg/kg, preferably 3.0 mg/kg may be employed, up to a maximum tolerated dose that was determined in one study to be 5.2 mg/kg. The texaphyrin is administered by intravenous injection, followed by a waiting period of from as short a time as several minutes or about 3 hours to as long as about 72 or 96 hours (depending on the treatment being effected) to facilitate intracellular uptake and clearance from the plasma and extracellular matrix prior to the administration of photoirradiation.

The co-administration of a sedative (e.g., benzodiazapenes) and narcotic analgesic are sometimes recommended prior to light treatment along with topical administration of Emla cream (lidocaine, 2.5% and prilocaine, 2.5%) under an occlusive dressing. Other intradermal, subcutaneous and topical anesthetics may also be employed as necessary to reduce discomfort. Subsequent treatments can be provided after approximately 21 days. The treating physician may choose to be particularly cautious in certain circumstances and advise that certain patients avoid bright light for about one week following treatment.

When employing photodynamic therapy, a target area is treated with light at about 732±16.5 nm (full width half max) delivered by LED device or an equivalent light source (e.g., a Quantum Device Qbeam™ Q BMEDXM-728 Solid State Lighting System, which operates at 728 nm) at an intensity of 75 mW/cm² for a total light dose of 150 J/cm². The light treatment takes approximately 33 minutes.

The optimum length of time following texaphyrin administration until light treatment can vary depending on the mode of administration, the form of administration, and the type of target tissue. Typically, the texaphyrin persists for a period of minutes to hours, depending on the texaphyrin, the formulation, the dose, the infusion rate, as well as the type of tissue and tissue size.

After the photosensitizing texaphyrin has been administered, the tissue being treated is photoirradiated at a wavelength similar to the absorbance of the texaphyrin, usually either about 400-500 nm or about 700-800 nm, more preferably about 450-500 nm or about 710-760 nm, or most preferably about 450-500 nm or about 725-740 nm. The light source may be a laser, a light-emitting diode, or filtered light from, for example, a xenon lamp; and the light may be administered topically, endoscopically, or interstitially (via, e.g., a fiber optic probe). Preferably, the light is administered using a slit-lamp delivery system. The fluence and irradiance during the photoirradiating treatment can vary depending on type of tissue, depth of target tissue, and the amount of overlying fluid or blood. For example, a total light energy of about 100 J/cm² can be delivered at a power of 200 mW to 250 mW depending upon the target tissue.

One aspect of the present invention is that the compounds of the present invention can additionally be used to image the localization of the therapeutic agent. The texaphyrin core allows for the use of MRI to determine the location of the compound with the patient and determine the specific location and margin of the tumor to which it has localized. In some aspects, the ability to determine the location of the texaphyrin core may be advantageous for more or additional therapeutic methods such as surgery or radiation therapy.

### D. Definitions

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanate” means —N═C═O; “azido” means —N₃; in a monovalent context “phosphate” means —OP(O)(OH)₂ or a deprotonated form thereof in a divalent context “phosphate” means —OP(O)(OH)O— or a deprotonated form thereof “mercapto” means —SH; and “thio” means ═S; “sulfonyl” means —S(O)₂—; and “sulfinyl” means —S(O)—.

In the context of chemical formulas, the symbol “—” means a single bond, “═” means a double bond, and “≡” means triple bond. The symbol “ [symbol] ” represents an optional bond, which if present is either single or double. The symbol “ [symbol] ” represents a single bond or a double bond. Thus, the formula

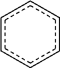

covers, for example,

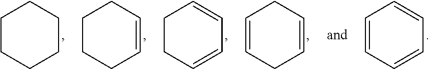

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.

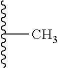

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a group “R” is depicted as a “floating group” on a ring system, for example, in the formula:

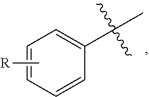

then R may replace any hydrogen atom attached to any of the ring atoms, including a depicted, implied, or expressly defined hydrogen, so long as a stable structure is formed. When a group “R” is depicted as a “floating group” on a fused ring system, as for example in the formula:

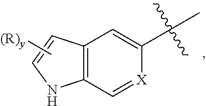

then R may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the group “R” enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” defines the exact number (n) of carbon atoms in the group/class. “Cn” defines the maximum number (n) of carbon atoms that can be in the group/class, with the minimum number as small as possible for the group/class in question, e.g., it is understood that the minimum number of carbon atoms in the group “alkenyl_{(C≤8)}” or the class “alkene_{(C≤8)}” is two. Compare with “alkoxy_{(C≤10)}”, which designates alkoxy groups having from 1 to 10 carbon atoms. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefin_{(C5)}”, and “olefin_{C5}” are all synonymous. When any of the chemical groups or compound classes defined herein is modified by the term “substituted”, any carbon atom(s) in a moiety replacing a hydrogen atom is not counted. Thus methoxyhexyl, which has a total of seven carbon atoms, is an example of a substituted alkyl_{(C1-6)}.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of ketoenol tautomerism or imine/enamine tautomerism are not precluded. When the term “saturated” is used to modify a solution of a substance, it means that no more of that substance can dissolve in that solution.

The term “aliphatic” when used without the “substituted” modifier signifies that the compound or chemical group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “alkyl” when used without the “substituted” modifier refers to a monovalent saturated aliphatic group with a carbon atom as the point of attachment, a linear or branched acyclic structure, and no atoms other than carbon and hydrogen. The groups —CH₃ (Me), —CH₂CH₃ (Et), CH₂CH₂CH₃ (n-Pr or propyl), —CH(CH₃)₂ (i-Pr, Tr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tent-butyl, t-butyl, t-Bu or ^{t}Bu), and —CH₂C(CH₃)₃ (neo-pentyl) are non-limiting examples of alkyl groups. The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” when used without the “substituted” modifier refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H—R, wherein R is alkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The following groups are non-limiting examples of substituted alkyl groups: —CH₂OH, —CH₂Cl, —CF₃, —CH₂CN, CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups.

The term “cycloalkyl” when used without the “substituted” modifier refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” when used without the “substituted” modifier refers to a divalent saturated aliphatic group with two carbon atoms as points of attachment, no carbon-carbon double or triple bonds, and no atoms other than carbon and hydrogen. The group

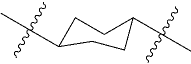 [SMILES: *C1CCC(*)CC1]

is a non-limiting example of cycloalkanediyl group. A “cycloalkane” refers to the class of compounds having the formula H—R, wherein R is cycloalkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkenyl” when used without the “substituted” modifier refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and CH═CHCH═CH₂. The term “alkenediyl” when used without the “substituted” modifier refers to a divalent unsaturated aliphatic group, with two carbon atoms as points of attachment, a linear or branched, a linear or branched acyclic structure, at least one nonaromatic carbon-carbon double bond, no carbon-carbon triple bonds, and no atoms other than carbon and hydrogen. The groups —CH═CH, —CH═C(CH₃)CH₂, —CH═CHCH₂—, and —CH₂CH═CHCH₂are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —CH═CHF, —CH═CHCl and —CH═CHBr are non-limiting examples of substituted alkenyl groups.

The term “cycloalkenyl” when used without the “substituted” modifier refers to a monovalent unsaturated aliphatic group with a carbon atom as the point of attachment, said carbon atom forming part of one or more non-aromatic ring structures, one or more carbon-carbon double bonds provided that the compound is not aromatic, no carbon-carbon triple bonds, and no atoms other than carbon and hydrogen. Non-limiting examples include: cyclopentene or cyclohexene. As used herein, the term does not preclude the presence of one or more alkyl groups (carbon number limitation permitting) attached to a carbon atom of the non-aromatic ring structure. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkynyl” when used without the “substituted” modifier refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C═CH, —C═CCH₃, and —CH₂C═CCH₃ are non-limiting examples of alkynyl groups. An “alkyne” refers to the class of compounds having the formula H—R, wherein R is alkynyl. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aryl” when used without the “substituted” modifier refers to a monovalent unsaturated aromatic group with an aromatic carbon atom as the point of attachment, said carbon atom forming part of a one or more aromatic ring structure, wherein the ring atoms are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” when used without the “substituted” modifier refers to a divalent aromatic group with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

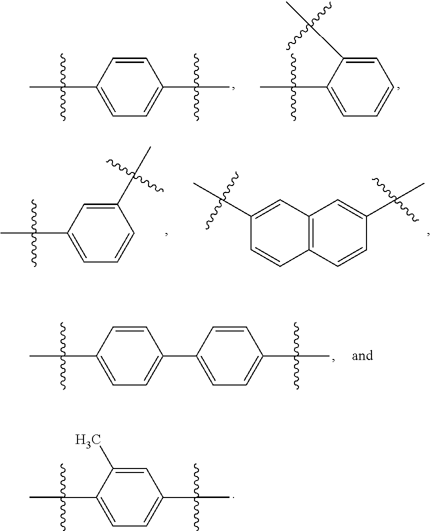

An “arene” refers to the class of compounds having the formula H—R, wherein R is aryl as that term is defined above. Benzene and toluene are non-limiting examples of arenes. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aralkyl” when used without the “substituted” modifier refers to the monovalent group alkanediylaryl, in which the terms alkanediyl and aryl are each used in a manner consistent with the definitions provided above. Non-limiting examples are: phenylmethyl (benzyl, Bn) and 2-phenyl-ethyl. When the term aralkyl is used with the “substituted” modifier one or more hydrogen atom from the alkanediyl and/or the aryl group has been independently replaced by —OH, —F, —Cl, —Br, —I, NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. Non-limiting examples of substituted aralkyls are: (3-chlorophenyl)-methyl, and 2-chloro-2-phenyl-eth-1-yl.

The term “heteroaryl” when used without the “substituted” modifier refers to a monovalent aromatic group with an aromatic carbon atom or nitrogen atom as the point of attachment, said carbon atom or nitrogen atom forming part of one or more aromatic ring structures wherein at least one of the ring atoms is nitrogen, oxygen or sulfur, the aromatic ring structures being one, two, three, or four ring structures each containing from three to nine ring atoms, and wherein the heteroaryl group consists of no atoms other than carbon, hydrogen, aromatic nitrogen, aromatic oxygen and aromatic sulfur. If more than one ring is present, the rings may be fused or unfused. Unfused rings are connected with a covalent bond. As used herein, the term heteroaryl does not preclude the presence of one or more alkyl or aryl groups (carbon number limitation permitting) attached to the aromatic ring or aromatic ring system. Non-limiting examples of heteroaryl groups include furanyl, imidazolyl, indolyl, indazolyl (Im), isoxazolyl, methylpyridinyl, oxazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the class of compounds having the formula H—R, wherein R is heteroaryl. Pyridine and quinoline are non-limiting examples of heteroarenes. When these terms are used with the “substituted” modifier one or more hydrogen atom has been independently replaced by —OH, —F, —Cl, —Br, —I, NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “heterocycloalkyl” when used without the “substituted” modifier refers to a monovalent non-aromatic group with a carbon atom or nitrogen atom as the point of attachment, said carbon atom or nitrogen atom forming part of one or more non-aromatic ring structures wherein at least one of the ring atoms is nitrogen, oxygen or sulfur, the non-aromatic ring structures being one, two, three, or four ring structures each containing from three to nine ring atoms, and wherein the heterocycloalkyl group consists of no atoms other than carbon, hydrogen, nitrogen, oxygen and sulfur. If more than one ring is present, the rings may be fused or unfused. As used herein, the term does not preclude the presence of one or more alkyl groups (carbon number limitation permitting) attached to the ring or ring system. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. N-pyrrolidinyl is an example of such a group. When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “acyl” when used without the “substituted” modifier refers to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C₆H₅, and —C(O)C₆H₄CH₃ are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkyl group, as defined above, attached to a —CHO group. When any of these terms are used with the “substituted” modifier one or more hydrogen atom (including a hydrogen atom directly attached to the carbon atom of the carbonyl or thiocarbonyl group, if any) has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkoxy” when used without the “substituted” modifier refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), or —OC(CH₃)₃ (tert-butoxy). The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” when used without the “substituted” modifier refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, N(CH₃)₂, —C(O)NH₂, —C(O)NHCH₃, —C(O)N(CH₃)₂, —OC(O)CH₃, NHC(O)CH₃, —S(O)₂OH, or —S(O)₂NH₂.

The term “alkylamino” when used without the “substituted” modifier refers to the group —NHR, in which R is an alkyl, as that term is defined above. Non-limiting examples include: —NHCH₃ and —NHCH₂CH₃. The term “dialkylamino” when used without the “substituted” modifier refers to the group —NRR′, in which R and R′ can be the same or different alkyl groups, or R and R′ can be taken together to represent an alkanediyl. Non-limiting examples of dialkylamino groups include: —N(CH₃)₂ and —N(CH₃)(CH₂CH₃). The terms “cycloalkylamino”, “alkenylamino”, “alkynylamino”, “arylamino”, “aralkylamino”, “heteroarylamino”, “heterocycloalkylamino”, “alkoxyamino”, and “alkylsulfonylamino” when used without the “substituted” modifier, refers to groups, defined as —NHR, in which R is cycloalkyl, alkenyl, alkynyl, aryl, aralkyl, heteroaryl, heterocycloalkyl, alkoxy, and alkylsulfonyl, respectively. A non-limiting example of an arylamino group is —NHC₆H₅. The term “amido” (acylamino), when used without the “substituted” modifier, refers to the group —NHR, in which R is acyl, as that term is defined above. A non-limiting example of an amido group is —NHC(O)CH₃. The term “alkylimino” when used without the “substituted” modifier refers to the divalent group ═NR, in which R is an alkyl, as that term is defined above. When any of these terms is used with the “substituted” modifier one or more hydrogen atom attached to a carbon atom has been independently replaced by —OH, —F, —Cl, —Br, —I, —NH₂, —NO₂, —CO₂H, —CO₂CH₃, —CN, —SH, —OCH₃,—OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —NHC(O)OCH₃ and —NHC(O)NHCH₃ are non-limiting examples of substituted amido groups.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects.

An “active ingredient” (AI) (also referred to as an active compound, active substance, active agent, pharmaceutical agent, agent, biologically active molecule, or a therapeutic compound) is the ingredient in a pharmaceutical drug or a pesticide that is biologically active. The similar terms active pharmaceutical ingredient (API) and bulk active are also used in medicine, and the term active substance may be used for pesticide formulations.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating or preventing a disease, is an amount sufficient to effect such treatment or prevention of the disease.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose, which is 50% of the maximum response obtained. This quantitative measure indicates how much of a particular active pharmaceutical ingredient or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “ligand” references to a chemical group which coordinates to a metal center through a bond. The bond between the ligand and the metal center in some cases is either an ionic or a coordination bond. A ligand can be monovalent, divalent, trivalent or have a greater valency. In some cases, a ligand may be negatively charged. Some exemplary examples of ligands include, but are not limited to, halide (F⁻, Cl⁻, Br⁻, or I⁻), a carbonate (CO₃²⁻), bicarbonate (HCO₃⁻), hydroxide (⁻OH), perchlorate (ClO₄⁻), nitrate (NO₃⁻), sulfate (SO₄²⁻), acetate (CH₃CO₂⁻), trifluoroacetate (CF₃CO₂⁻), acetylacetonate (CH₃COCHCOCH₃⁻), trifluorosulfonate (CF₃SO₂⁻), phosphate (PO₄³⁻), oxalate, ascorbate, or gluconate. A ligand could also be a neutral species that contains a lone pair of electrons. Some examples of neutral ligands include but are not limited to aqua (H₂O) or ammonia (NH₃). Additionally, a neutral ligand can include groups such as an alkylamine or a dialkylamine.

As used herein, the term “patient” or “subject” refers to a living mammalian organism, such as a human, monkey, cow, horse, sheep, goat, dog, cat, mouse, rat, guinea pig, or transgenic species thereof. In certain embodiments, the patient or subject is a primate. Non-limiting examples of human patients are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hy droxy-2-ene-1-carboxylic acid), 4-methylbicyclo[2.2.2]oct-2-ene-l-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

A “pharmaceutical drug” (also referred to as a pharmaceutical, pharmaceutical preparation, pharmaceutical composition, pharmaceutical formulation, pharmaceutical product, medicinal product, medicine, medication, medicament, or simply a drug) is a compound or composition used to diagnose, cure, treat, or prevent disease. An active ingredient (AI) (defined above) is the ingredient in a pharmaceutical drug or a pesticide that is biologically active. The similar terms active pharmaceutical ingredient (API) and bulk active are also used in medicine, and the term active substance may be used for pesticide formulations. Some medications and pesticide products may contain more than one active ingredient. In contrast with the active ingredients, the inactive ingredients are usually called excipients (defined above) in pharmaceutical contexts.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an active pharmaceutical ingredient according to the present invention. The prodrug itself may or may not also have activity with respect to a given target protein. For example, a compound comprising a hydroxy group may be administered as an ester that is converted by hydrolysis in vivo to the hydroxy compound. Suitable esters that may be converted in vivo into hydroxy compounds include acetates, citrates, lactates, phosphates, tartrates, malonates, oxalates, salicylates, propionates, succinates, fumarates, maleates, methylene-bis-β-hydroxynaphthoate, gentisates, isethionates, di-p-toluoyltartrates, methanesulfonates, ethanesulfonates, benzenesulfonates, p-toluenesulfonates, cyclohexylsulfamates, quinates, esters of amino acids, and the like. Similarly, a compound comprising an amine group may be administered as an amide that is converted by hydrolysis in vivo to the amine compound.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

The term “unit dose” refers to a formulation of the compound or composition such that the formulation is prepared in a manner sufficient to provide a single therapeutically effective dose of the active ingredient to a patient in a single administration. Such unit dose formulations that may be used include but are not limited to a single tablet, capsule, or other oral formulations, or a single vial with a syringable liquid or other injectable formulations.

The above definitions supersede any conflicting definition in any reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### E. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1: Disulfide Linked Texaphyrin and Doxorubicin Conjugate

Conjugate 1 is comprised of doxorubicin (Dox), an antitumor inhibitor of topoisomerase II, a disulfide linker that is readily cleaved by thiols, such as glutathione (GSH), that are relatively abundant in tumor cells, and a Gd³⁺-texaphyrin complex, which serves as an MRI contrast agent. Conjugate 1 was prepared according to the synthetic route outlined in Scheme 1. Briefly, MGd was converted to the monoamino derivative 3 via 2 in accord with previously published procedures (Wei et al., 2005). Precursor 3 was then reacted with the disulfide linker component, 6, in the presence of DIPEA to give 4, which was then treated with 4-nitrophenyl chloroformate and DIPEA, followed by doxorubicin (Dox) and DIPEA in DMF, to produce 5. Acid-mediated deprotection then gave the texaphyrin-disulfide-doxorubicin conjugate 1. A texaphyrin-doxorubicin conjugate, 10, containing a CH₂CH₂ bridge instead of the disulfide linker was prepared using a similar synthetic approach.

Once prepared, conjugate 1 was converted to a liposomal formulation by mixing with polyethylene glycol (PEG)-cholesterol, 1,2-dioleoyl-sn-gly cero-3-phosphoethanolamine (DOPE), 1,2-dioleoyl-sn-glycero-3-phosphoethanolamine-N-[methoxy(polyethylene glycol)-2000] (mPEG-DSPE), and 1,2-distearoyl-sn-glycero-3-phosphoethanolamine-N-[folate(polyethylene glycol)-2000] (folate-PEG-DSPE) at a molar ratio of 4:1:3:1.96:0.04. Evaporation, rehydration with 10 mM HEPES buffer, vortexing, and sonication for 10 min then yielded FL-1. A reference liposomal formulation, FL-10, made up from the control system 10, was produced in a similar way (Scheme 2). On the basis of dynamic light scattering studies, the size FL-1 was determined to be 119.97±3.16 nm. The zeta-potential was −12.93±0.32 mV. Similar values were recorded for FL-10.

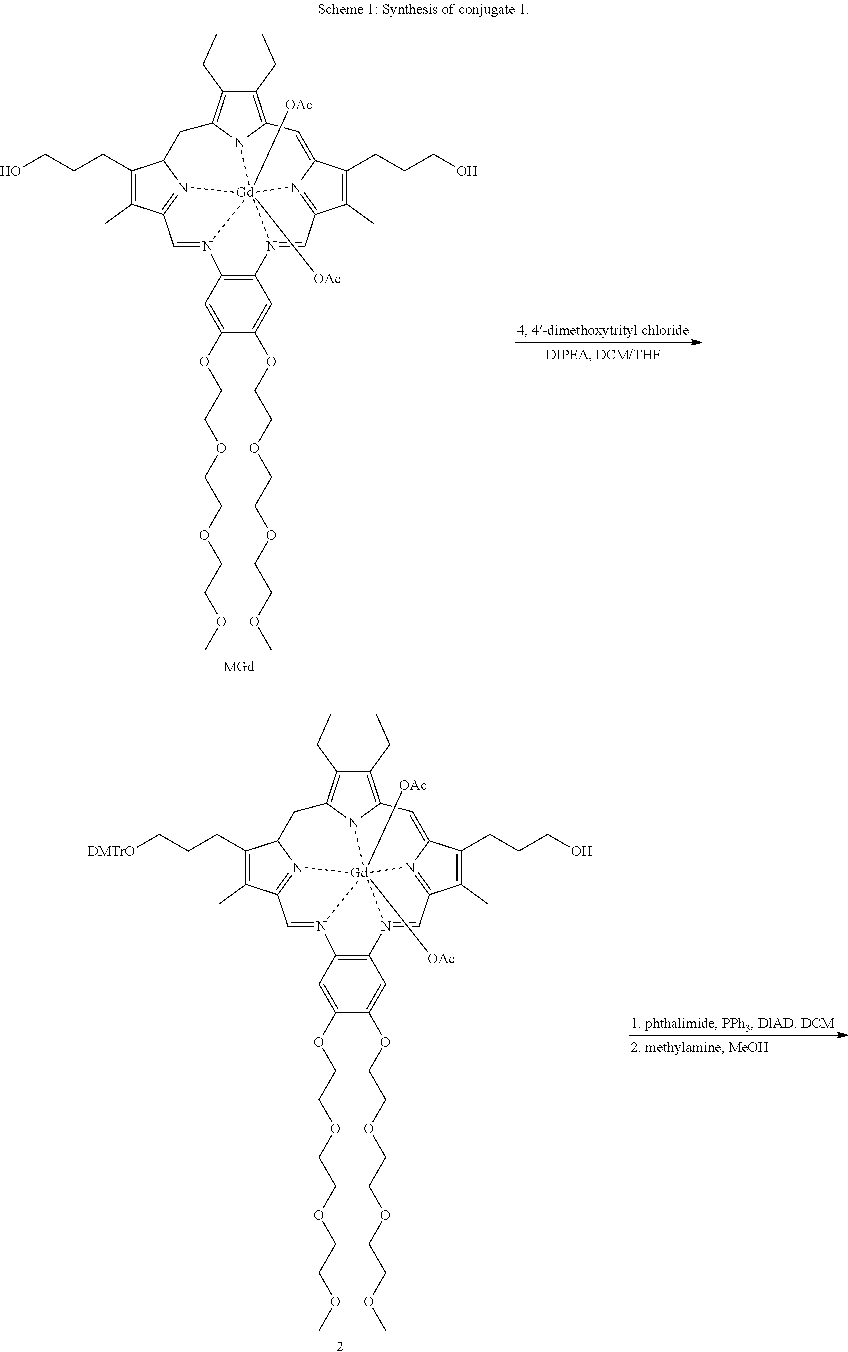

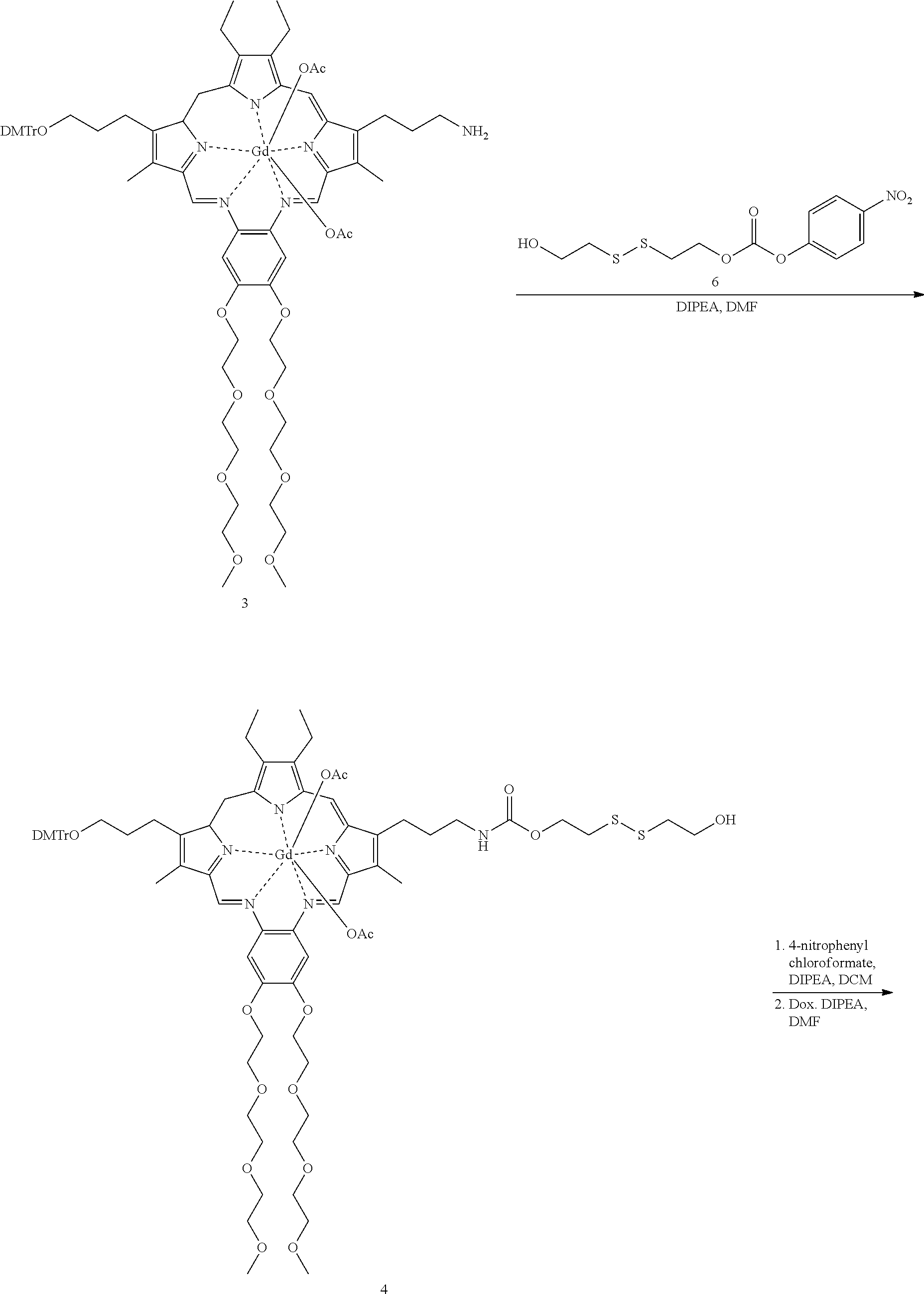

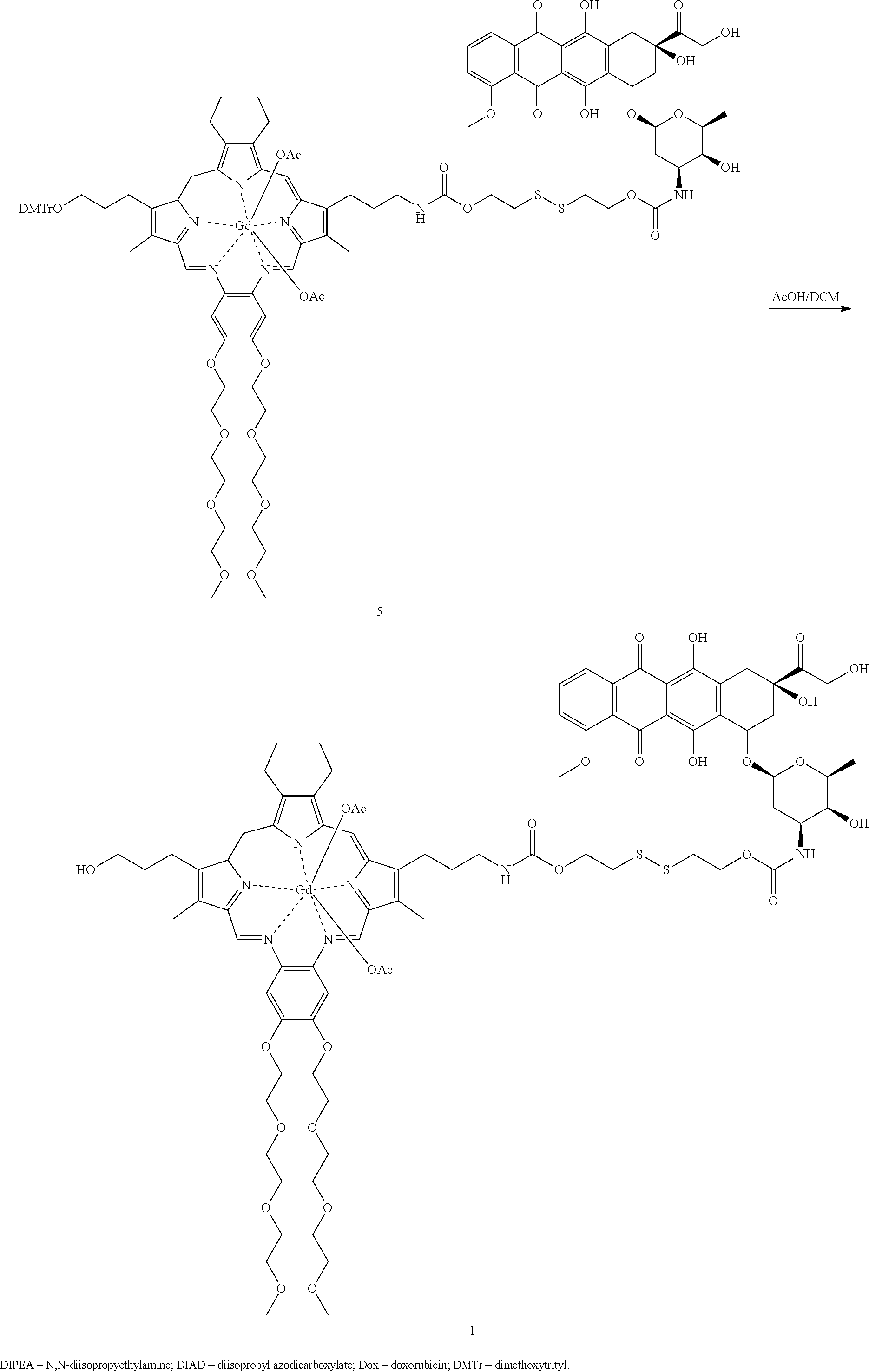

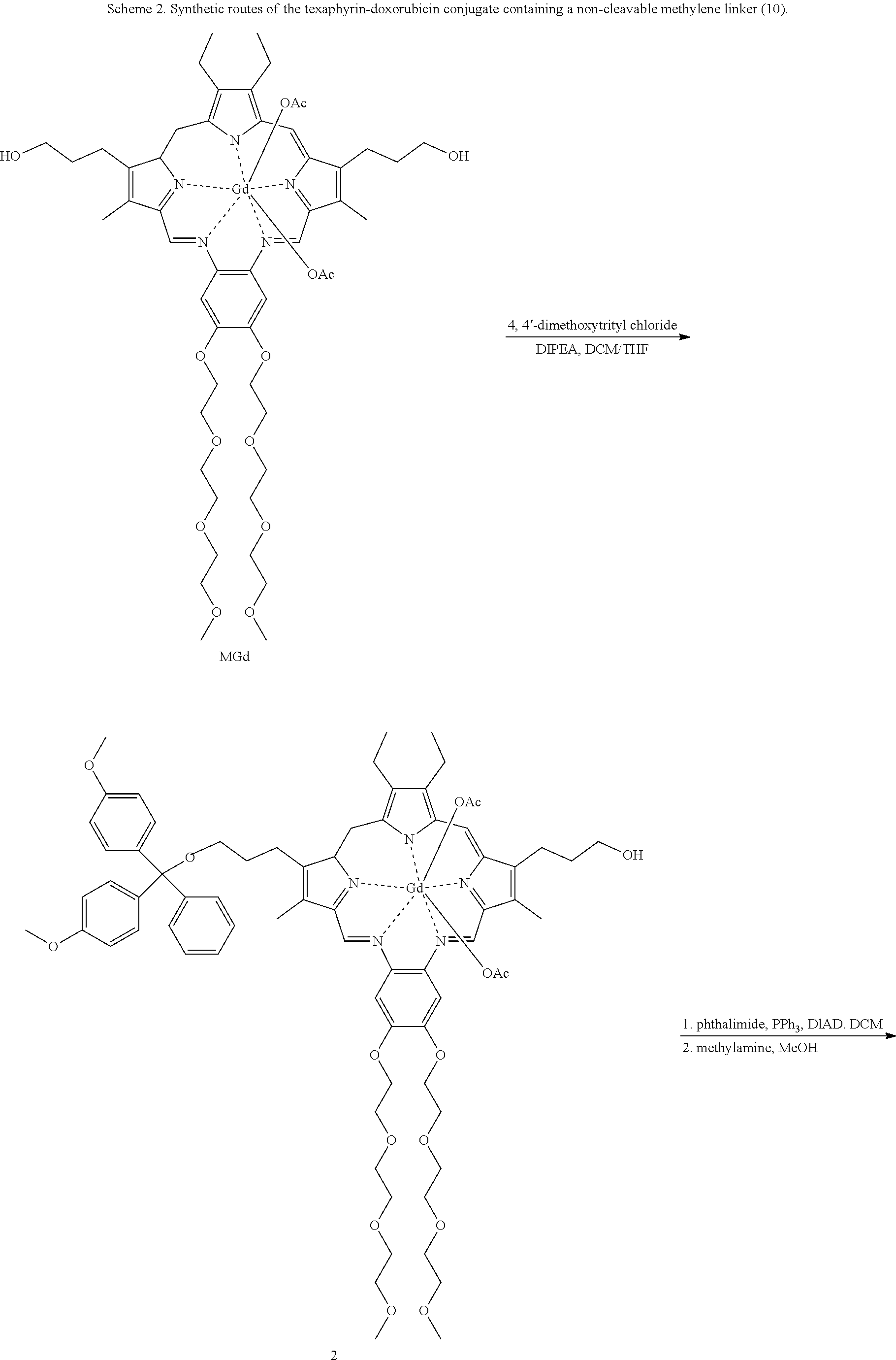

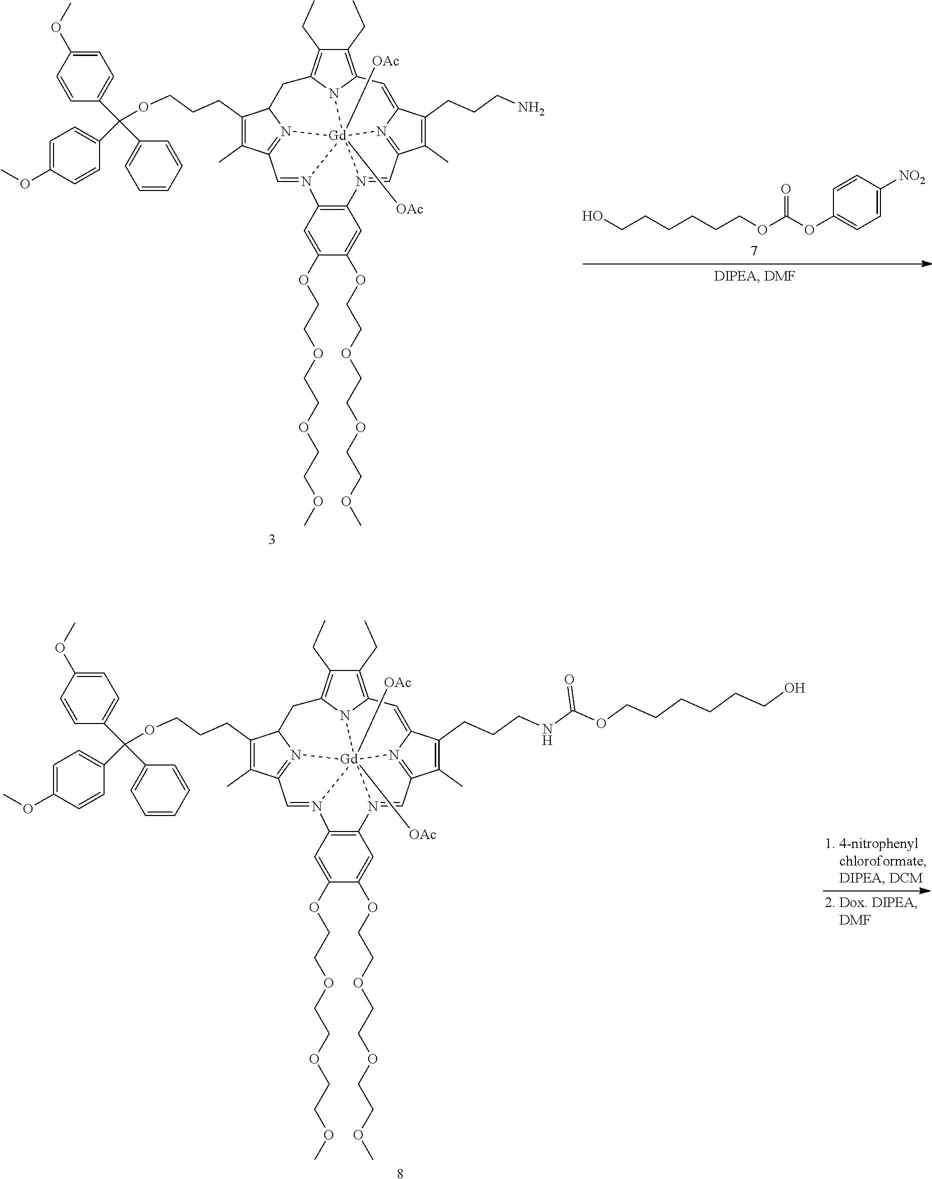

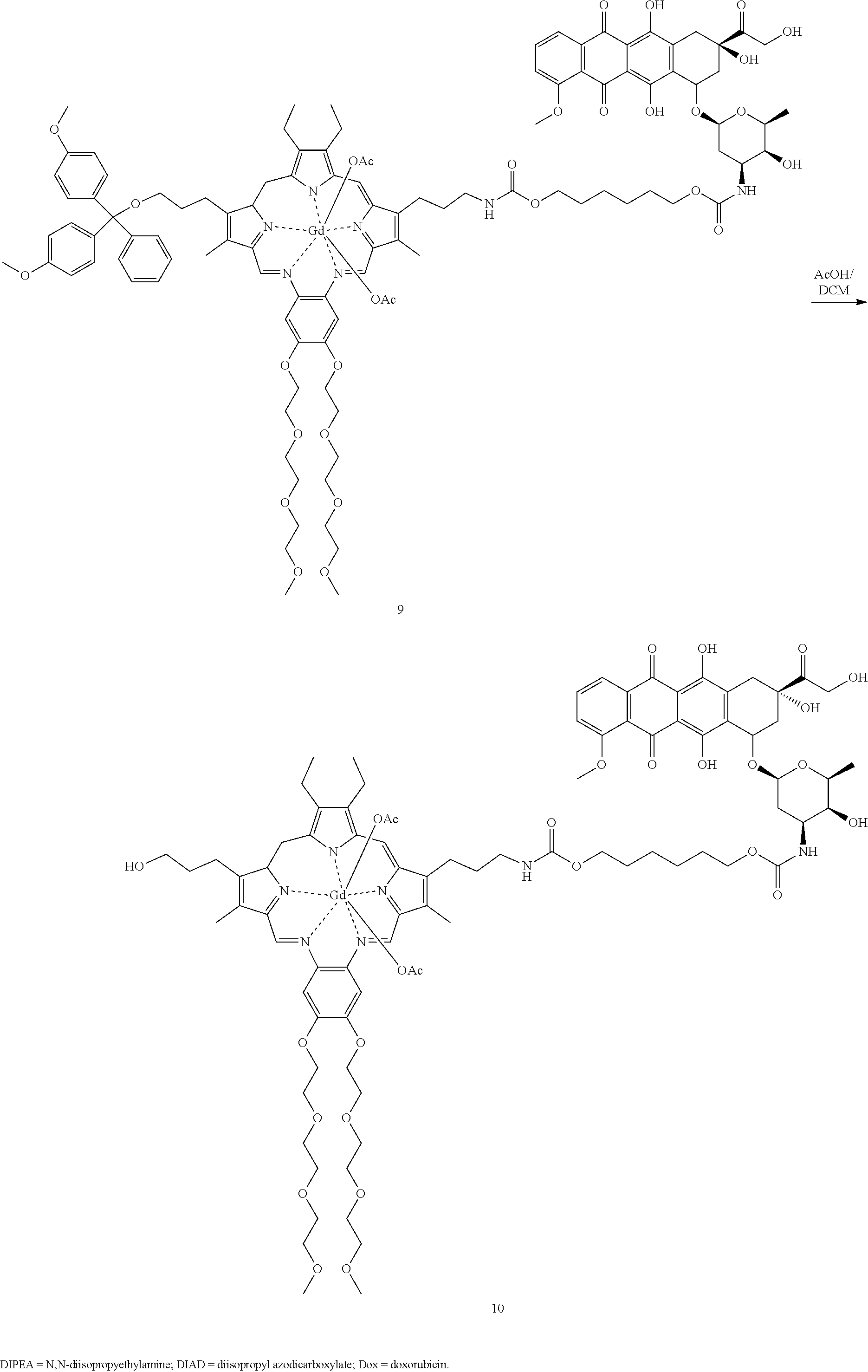

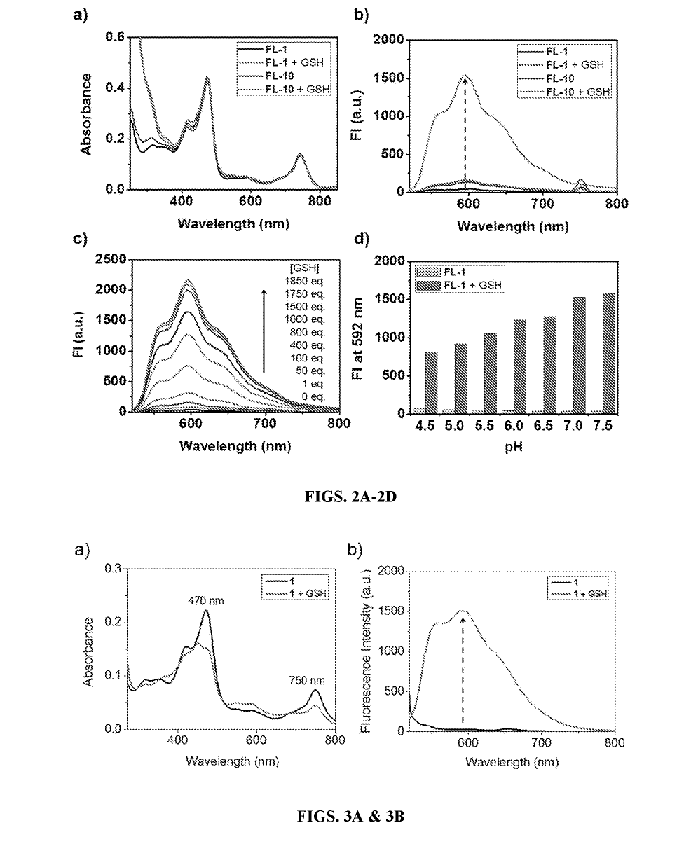

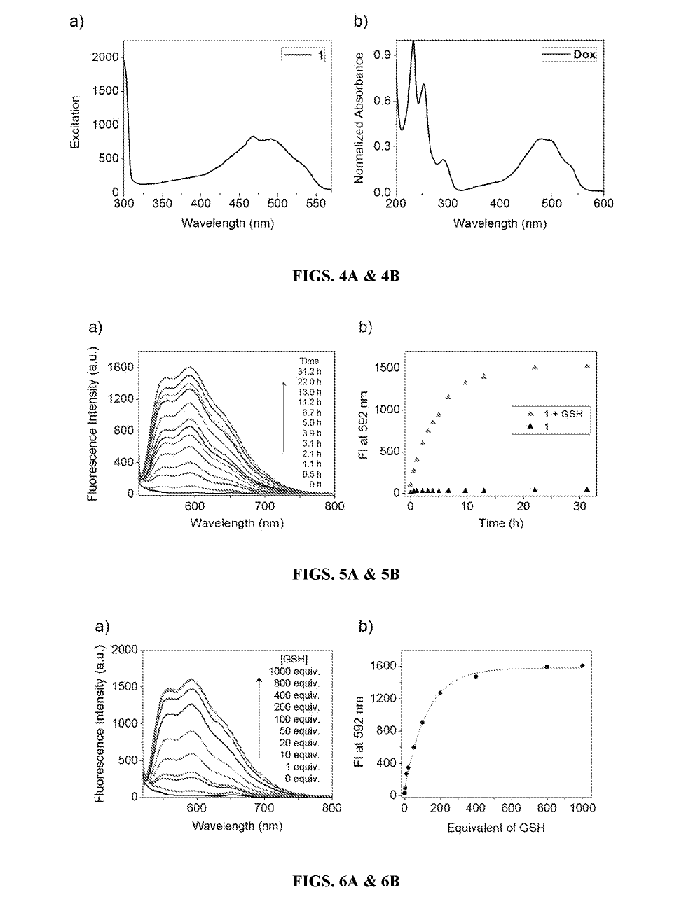

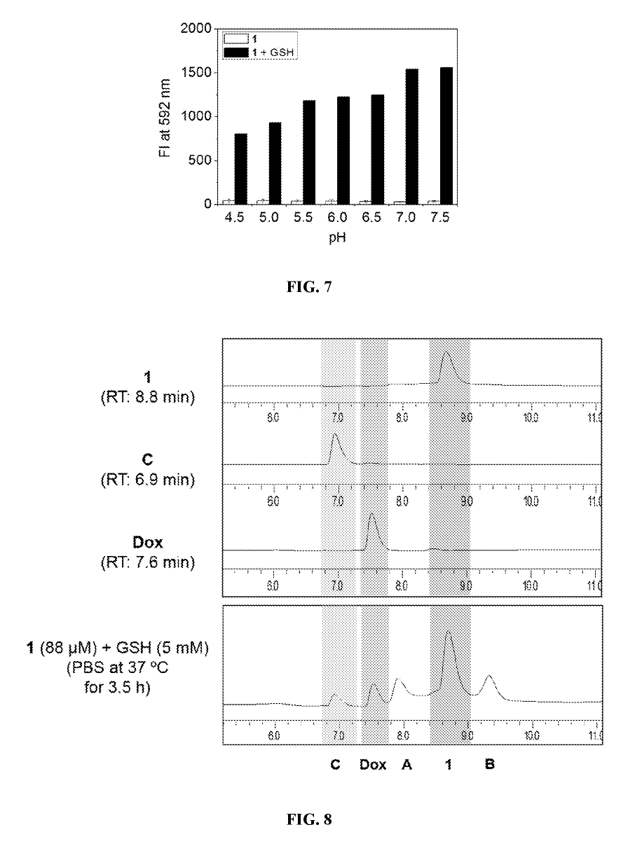

Evidence of GSH-induced cleavage came from fluorescence studies carried out in PBS (pH 7.4) at 37° C. As can be seen from an inspection of FIG. 2A, FL-1 displays a very weak fluorescence. However, when exposed to excess GSH (up to 1500 equiv) a ca. 38-fold increase in the fluorescence intensity at 592 nm is observed. This enhancement proved rather insensitive to pH over the 4.5-7.5 pH range. Analogous optical changes were seen for prodrug 1 under similar conditions (FIGS. 3-7). No appreciable changes were seen for either FL-10 or the control conjugate 10 when subject to identical testing. Further evidence for disulfide cleavage in the case of 1 came from HPLC and LC-mass spectrometry experiments (FIG. 8). A proposed mechanism for the GSH-induced disulfide cleavage is shown in Scheme 3.

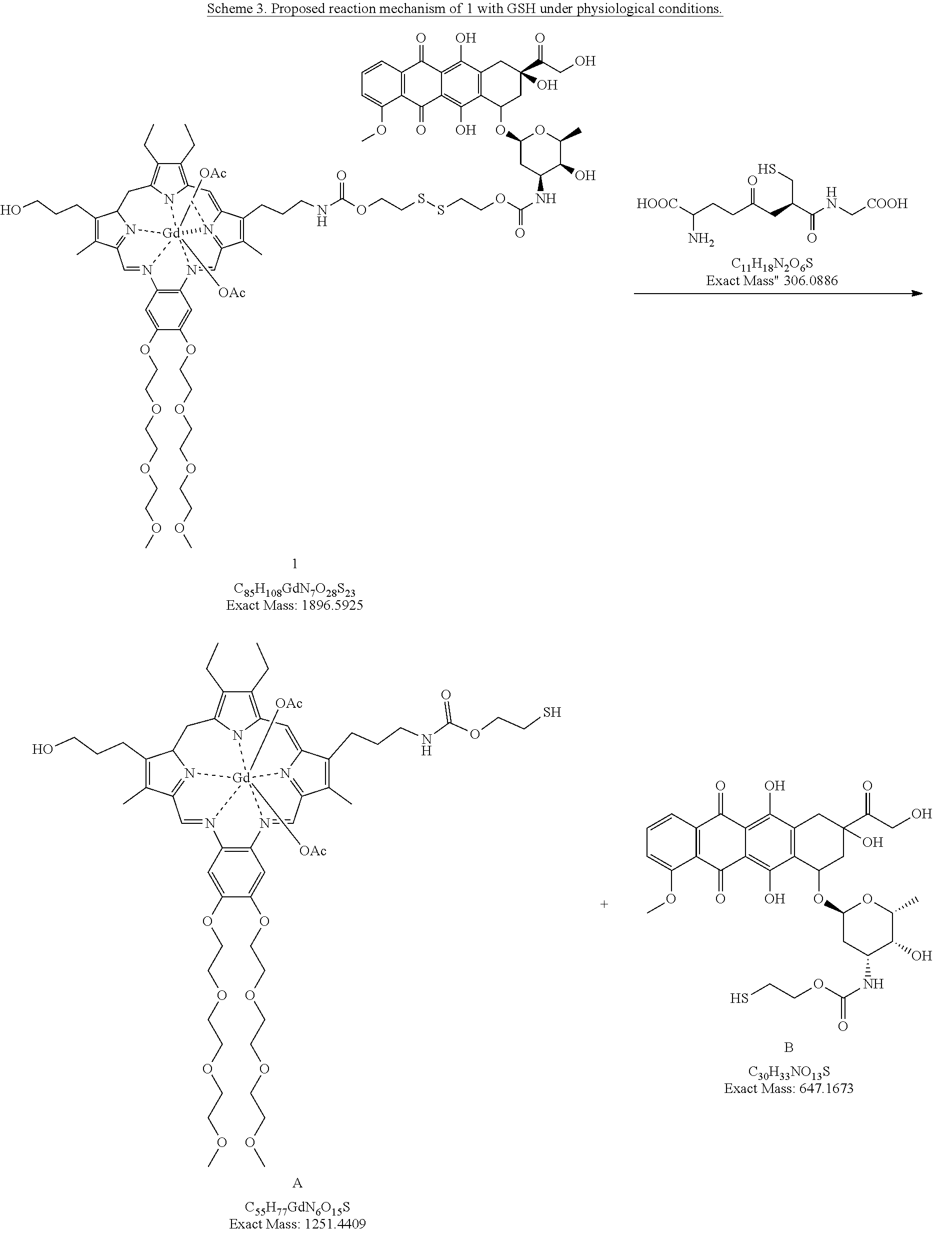

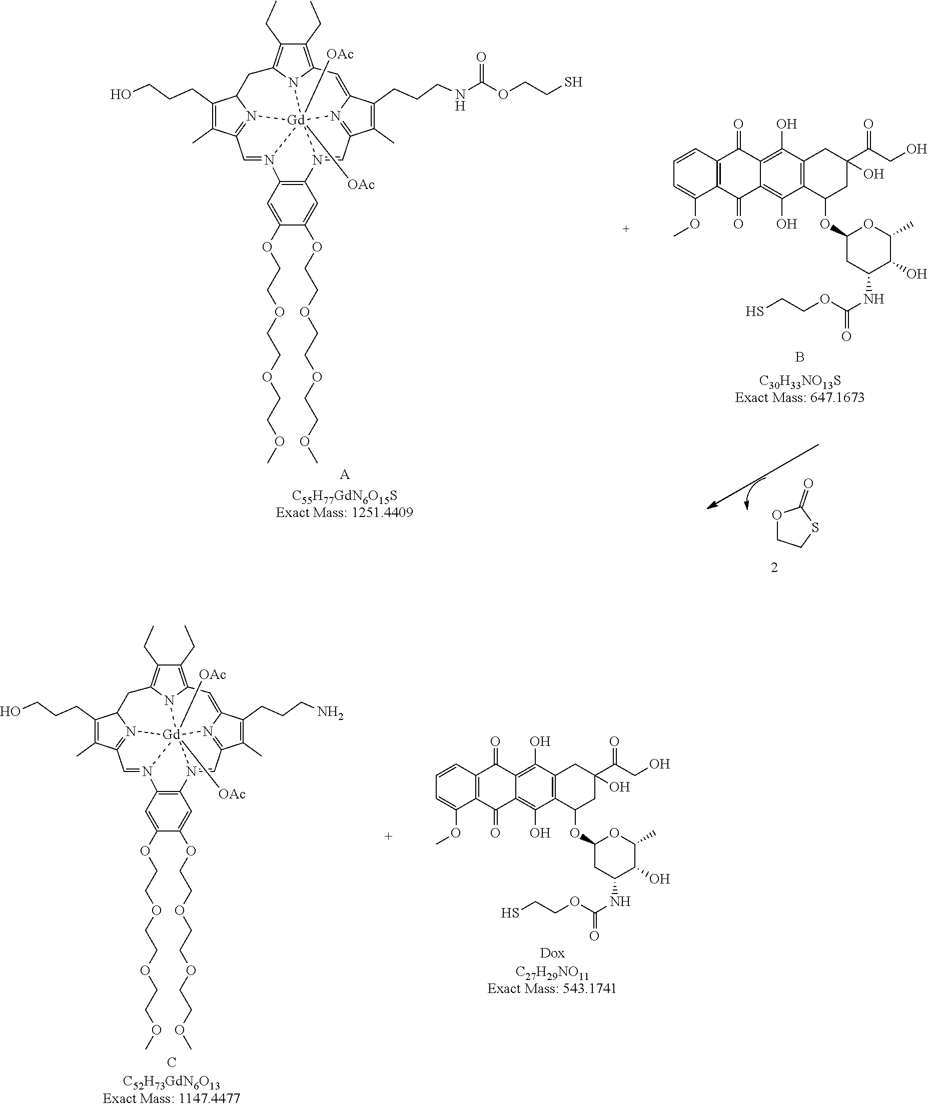

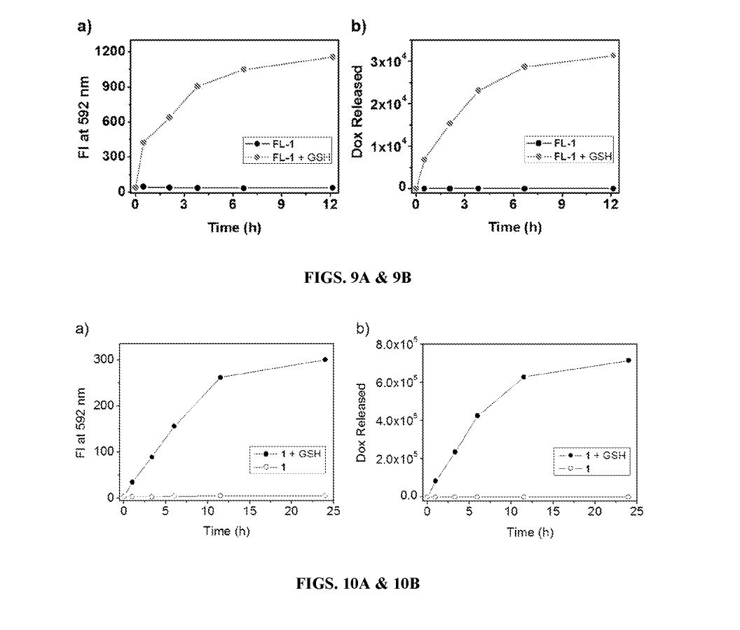

Further confirmation that upon disulfide bond cleavage, release of free Dox occurs came from combined HPLC and fluorimetric time-dependent analyses of FL-1 (FIGS. 9A & 9B). Upon treating FL-1 with GSH, the amount of Dox released was found to correlate with the observed increase in fluorescence intensity at 592 nm (arising from free Dox). In contrast, in the absence of GSH, little evidence of Dox release was seen by HPLC; nor, was an enhancement in the fluorescence intensity at 592 nm observed over the full 12 h time course of the experiment. Similar observations were made in the case of the prodrug conjugate 1 (FIGS. 10A & 10B). Without wishing to be bound by any theory, it is believed that the fluorescence changes at 592 nm provide an Off-On signal that may be used to follow directly Dox release.

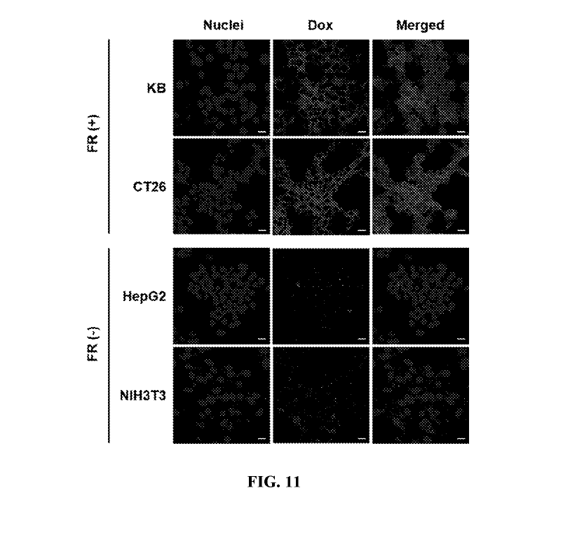

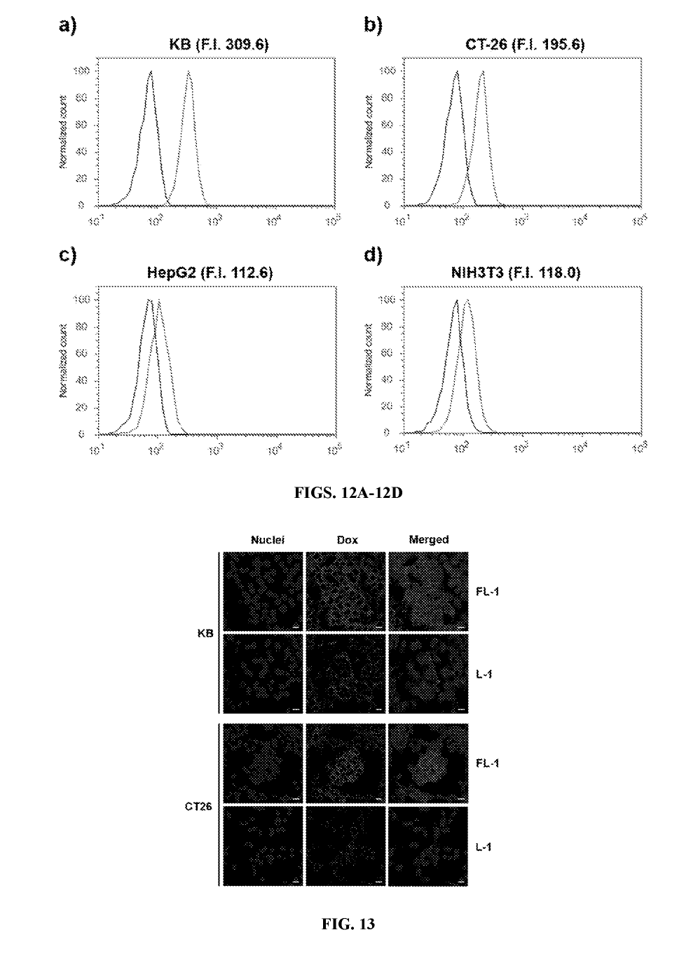

As the result of the folate-functionalized PEGylated liposomal formulation that makes it up, FL-1 was expected to provide for the tumor-targeted delivery of Dox via receptor-mediated endocytosis. Tests of this expectation were carried out using the KB and CT26 cell lines, which express folate receptors on the cell surface, as well as the HepG2 and NIH3T3 cell lines, which are folate receptor deficient. When the cells were treated with 4 μM of FL-1 for 1 h, strong fluorescence signals were observed in the folate receptor-positive cells, KB and CT26. On the other hand, only a very weak fluorescence signal was seen in the case of the HepG2 and NIH3T3 cells (FIG. 11). Support for the enhanced uptake via folate receptor targeting inferred from these optical studies came from histogram plots and quantitative fluorescence intensity measurements carried out via flow cytometry (FIGS. 12A-12D). Control liposomes (L-1), containing 1 but lacking the folate moieties, were then made up. The control liposomes were used to treat the folate receptor-expressing KB and CT26 cells in direct analogy to what was done in the case of FL-1. In this case, considerably lower levels of Dox uptake were observed, as inferred from comparative fluorescent microscopic imaging (FIG. 13). These results suggest that FL-1 permits the active targeted delivery of 1 into cancerous cells overexpressing the folate receptor and that Dox is released effectively within these cells.

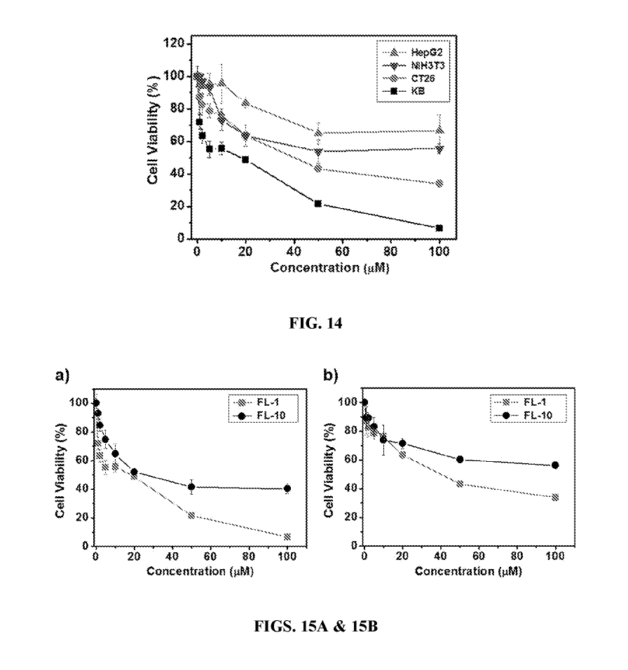

The anticancer effects of FL-1 were examined using standard MTT cell viability assays. Significant and moderate anti-proliferative activity was seen at 5 μM in the case of the KB and CT26 cell lines, respectively. Dose dependent effects were seen, with the activity increasing with concentration. At all concentrations, the activity was lower in the case of the HepG2 and NIH3T3 cell lines lacking the over-expressed folate receptors present in the KB and CT26 cell lines (FIG. 14).

The anti-proliferative activity of FL-1 is expected to reflect both liposome-based folate receptor targeting and Dox release via disulfide bond reduction. To test the importance of the latter factor, FL-10, a folate receptor targeted liposomal formulation loaded with control compound 10 containing a CH₂CH₂ unit instead of the S—S linker present in 1 was prepared. Treatment of the folate receptor-positive cell lines, KB and CT26, with this liposomal formulation resulted in a considerably lower anti-proliferative effect than seen for FL-1 (FIGS. 15A & 15B). The MTT assay results were supported by fluorescence measurements that revealed an increase in the Dox-based emission intensity. Without wishing to be bound by any theory, it is believed that the disulfide bond present in 1 (and FL-1) facilitates the release of free Dox.

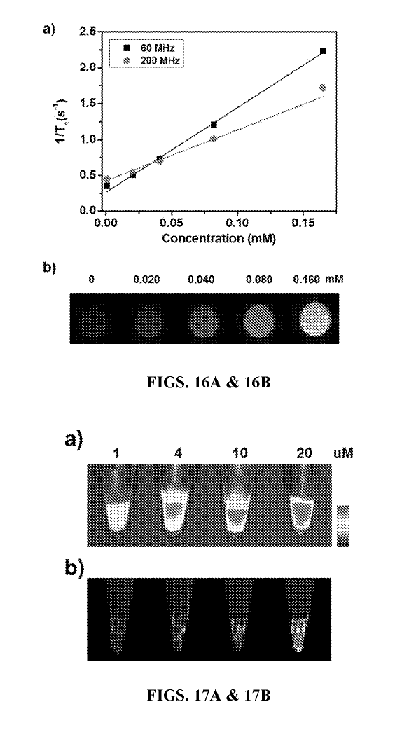

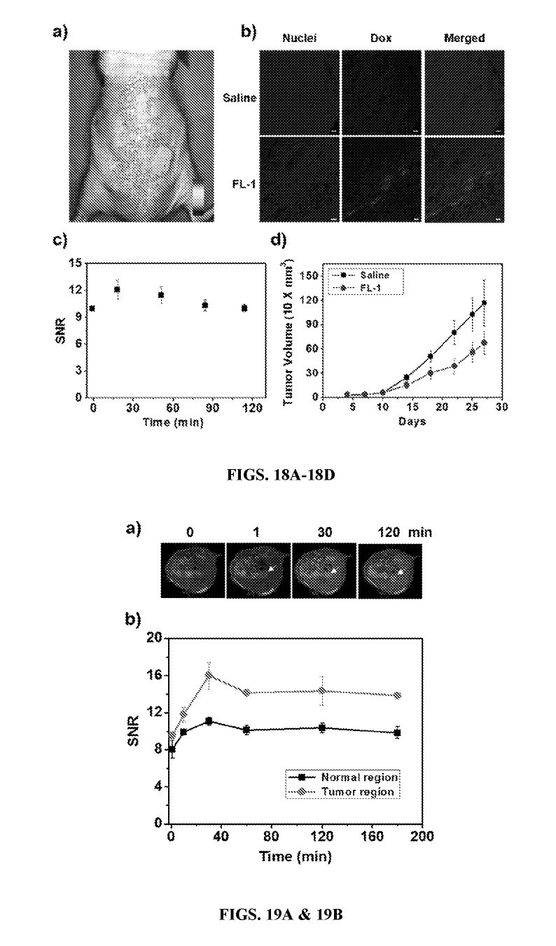

Conjugate 1 is expected to enhance T₁-weighted MR images through the coordinated Gd³⁺ center present in the texaphyrin core. Therefore, the MR relaxivity of FL-1 was examined in phosphate buffered saline. The T₁ relaxivities of FL-1 were calculated to be 11.8±0.3 and 7.1±0.4 mM⁻¹s⁻¹ at 60 and 200 MHz, respectively (FIG. 16A). Phantom images acquired at 200 MHz in PBS reveal increasingly bright signals as the concentration of FL-1 increases (FIG. 16B). It was also confirmed that pellets of KB cells treated with ≥10 μM concentrations of FL-1 could be visualized by fluorescence emission at well as by use of an MRI scanner (FIGS. 17A & 17B). These results are taken as evidence that FL-1 would provide sufficient T₁ relaxivity to enable MR visualization in vivo and that, upon linker scission, would allow based Dox fluorescence-based imaging.

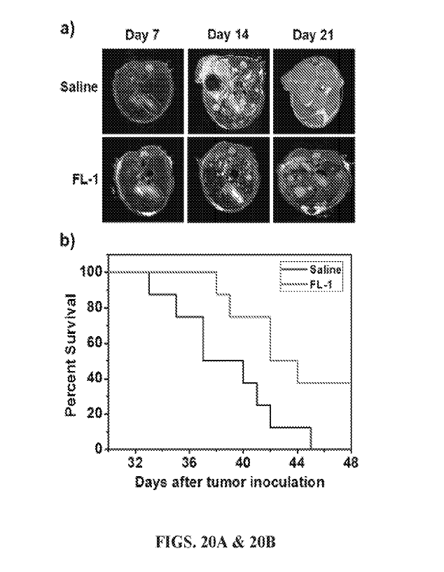

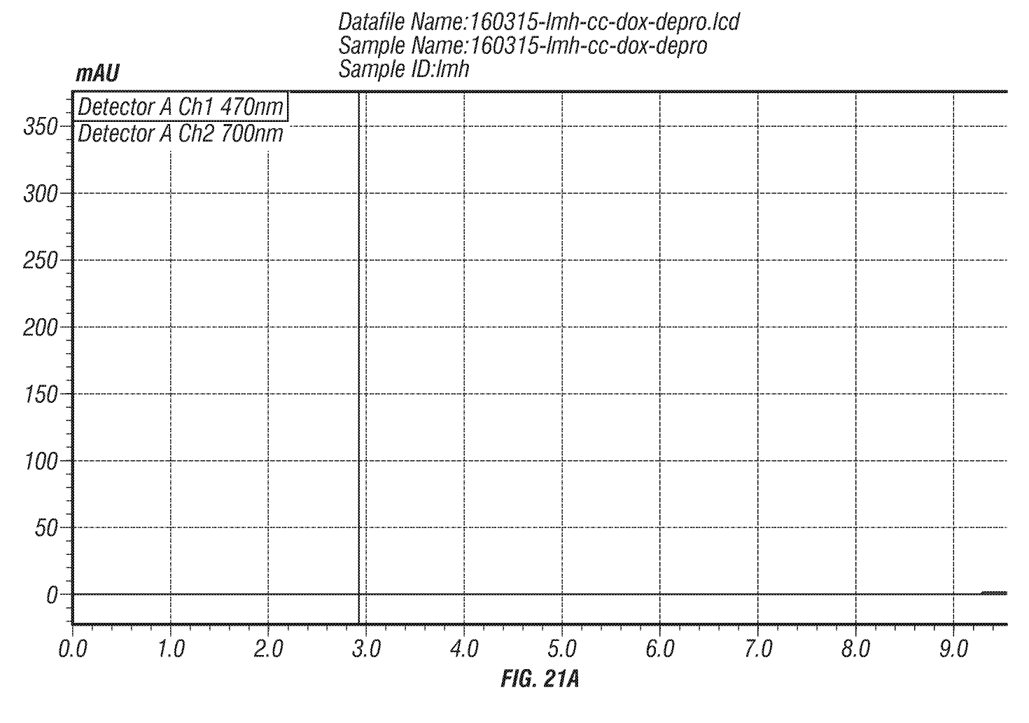

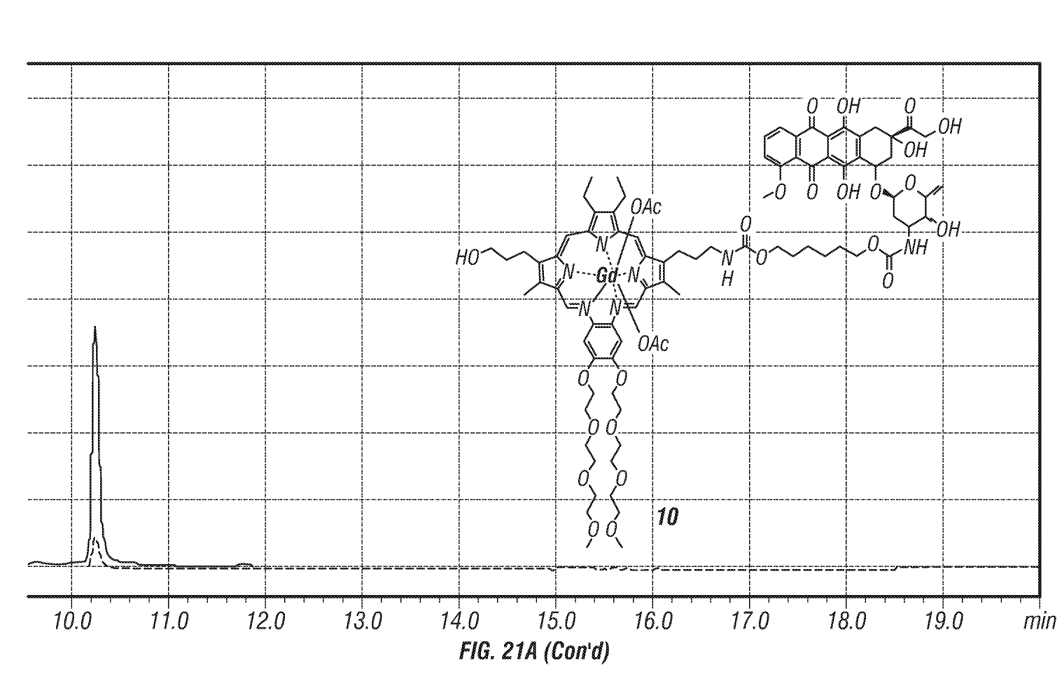

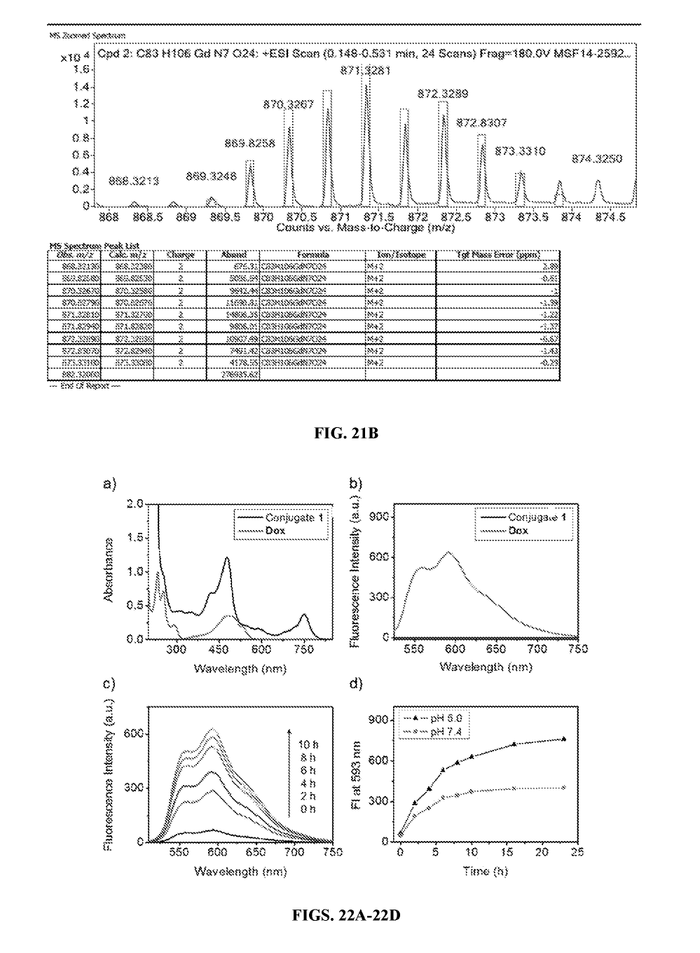

As a test of the above hypothesis, two different cancer mouse models, consisting of a metastatic liver cancer orthotropic model and a subcutaneous (S.C.) KB cell xenograft model, respectively, were used. First, FL-1 was evaluated to determine if FL-1 would accumulate in the tumor site and induce tumor regression in the subcutaneous xenograft nude mouse model. Here, in vivo whole-body fluorescence imaging revealed that a strong, tumor-localized signal 6 h after FL-1 was administered i.v. via tail vein injection (FIG. 18A). Cryo-sectioned tumor sections of animals treated with FL-1 were monitored after 24 h using confocal microscopy; again, strong enhancement was seen (FIG. 18B). Furthermore, good signal-to-noise ratios (SNRs) were seen for tumor tissues under conditions of T₁ MR imaging (FIG. 18C). The inferred localization provides a rationale for the relative reduction in tumor burden seen for FL-1 vs. saline control as determined from time dependent tumor size measurements (FIG. 18D).

The metastatic liver cancer model used for this study was obtained via the intrasplenic administration of CT26 cells to nude mice. As inferred from T₁-weighted MR images (FIG. 19A), FL-1 reveals effectively the tumor area, which is surrounded by normal liver tissue, and can do so at an early stage of metastatic disease (day 3 post-inoculation). Enhanced MR signals were seen in the tumor region as early as 30 min after i.v. administration (tail vein injection). The intensity of the signal decreased only gradually with time, presumably reflecting slow clearance of the conjugate from the tumor site (FIG. 19B).

To assess therapeutic efficacy in the metastatic liver cancer model, FL-1 (2.5 mg/kg) was administered intravenously in form of four doses. These doses were administered once every other day starting on the 3^{rd} day after inoculation with the CT26 cells used to produce the model. The extent of metastasis was monitored weekly starting on day 7 post inoculation using T₂-weighted MR imaging (FIGS. 20A & 20B). In all cases, a bright spot was seen at day 7 by T₂-weighted MRI, a finding ascribed to the initial migration of CT26 cells from the spleen into the liver. One week later, MR imaging revealed metastatic tumors scattered throughout the liver, with the extent of this dissemination being considerably greater in the case of the saline control (FIG. 20A). By day 21, the liver appeared fully invaded in the case of the saline control, whereas the metastases remained localized in the case of FL-1.

The survival rates between the saline control and the FL-1-treated group were compared using the metastatic liver model mice. A Kaplan-Meier analysis was carried out and revealed that the cumulative survival rates were enhanced for FL-1 relative to the saline control. No mice treated with saline survived past day 45. On the other hand, at day 45 post inoculation, 3 of the 8 mice treated with FL-1 were still alive (FIG. 20B). Two of the 8 mice treated with FL-1 survived to the end of the study (day 55).

### Example 2: Compound Characterization

The starting material (MGd), as well as compounds 2 and 3 were prepared using literature procedures (Sessler et al., 1999; Wei et al., 2005).

Compound 4: To a solution of 2-hydroxyethyl disulfide (2.3 g, 14.9 mmol) and N,N-diisopropyethylamine (DIPEA; 865 μL, 5.0 mmol) in distilled dichloromethane (DCM; 20 mL) in an ice bath, a solution of 4-nitrochloroformate (1.0 g, 5.0 mmol) dissolved in distilled DCM (10 mL) was slowly added. After stirring for 3 h, the solvent was evaporated under vacuum and the crude product obtained as a result was redissolved in ethyl acetate. The crude product was then purified by silica gel column chromatography using ethyl acetate and hexanes (v/v, 2:1) as the eluent. This gave product 4 as a colorless oil in 63% yield (1.0 g). ESI-MS m/z [M+Na]⁺ calc. 342.0076, obs. 342.0078. ¹-H NMR (CDCl₃, 400 MHz): δ8.81 (d, 1H, J=1.60 Hz); 8.45-8.42 (m, 1H); 8.24-8.22 (d, 2H, J=9.21 Hz); 7.93-7.91 (m, 1H); 7.45 (t, 2H, J=3.20 Hz); 7.05-7.03 (m, 1H); 6.33 (s, 2H); 6.18 (s, 2H); 3.55 (t, 2H, J=6.80 Hz); 3.48-3.45 (m, 4H); 3.22-3.16 (m, 4H); 1.82 (s, 6H); 1.33 (t, 6H, J=6.80 Hz). ¹³C NMR (CDCl₃, 400 MHz): 196.3, 168.5, 162.5, 156.4, 154.0, 151.9, 147.5, 136.3, 132.6, 131.2, 130.6, 128.7, 128.5, 128.2, 128.1, 123.9, 122.9, 118.0, 106.2, 105.2, 96.8, 77.7, 65.2, 59.1, 41.4, 38.5, 16.8, 14.9 ppm.

Compound 5: Compounds 3 (160 mg, 0.11 mmol) and 4 (39 mg, 0.12 mmol) were dissolved in anhydrous DMF (10 mL) in a flask chilled in an ice bath. N,N-Diisopropylethylamine (DIPEA) (93 μL, 0.52 mmol) was slowly added to the reaction mixture, which was then stirred overnight at room temperature. The progress of the reaction was monitored by HPLC. When the starting material 3 disappeared as inferred from the HPLC analysis, the reaction mixture was diluted with ammonium acetate buffer and loaded onto a C18 cartridge. The cartridge was then subjected to elution with an increasing gradient of CH₃CN (1090%) in an ammonium acetate buffer. The product, 5, eluted off the cartridge when the percentage of CH₃CN was 50˜55%. The fraction obtained in this way was then loaded on a new C18 cartridge, which was desalted with HPLC water and subject to elution with pure MeOH. The fraction collected in this way was subject to drying under vacuum; this gave product 5 as a green sticky solid in 65% yield (100 mg). ESI-MS m/z [M-2OAc]²⁺ calc. 755.7718, obs. 755.7734.

Compound 6: 4-Nitrochloroformate (4.0 mg, 0.18 mmol) was slowly added to a solution of 5 (20 mg, 0.01 mmol) and DIPEA (22 μL, 0.12 mmol) in distilled DCM (20 mL) in a flask chilled in an ice bath. The resulting reaction mixture was then stirred for 3 h. The progress of the reaction was monitored by HPLC. When the starting material 5 disappeared as inferred from the HPLC analysis, the solvent was evaporated off under vacuum. To the residue contained in a flask cooled in an ice bath, a solution of doxorubicin-HCl (8.5 g, 0.15 mmol) and DIPEA (16 μL, 0.12 mmol) in DMF was added. After stirring overnight, the reaction mixture was diluted with ammonium acetate buffer and loaded onto a C18 cartridge. This cartridge was then subjected to elution with an increasing gradient of CH₃CN (10˜90%) in an ammonium acetate buffer. The product 6 eluted off when the gradient consisted of 65% CH₃CN. The fraction collected in this way was then loaded onto a new C18 cartridge, which was desalted with HPLC water and then eluted with pure MeOH. The eluent was taken to dryness in vacuo to give the product 6 as a greenish brown solid in 37% yield (10 mg). ESI-MS m/z [M-2OAc-H+Na]²⁺ calc. 1051.3398, obs. 1051.3425.

Conjugate 1: Precursor 6 (67 mg) was dissolved in a mixture of DCM (0.5 mL) and AcOH (5 mL) and stirred for 6 h at room temperature. The solvent was then evaporated off under vacuum. The residue was dissolved in a mixture of CH₃CN/ammonium acetate buffer (v/v, 20/80) and loaded onto a C18 cartridge. This cartridge was then subjected to elution with an increasing gradient of CH₃CN (1090%) in an ammonium acetate buffer. Conjugate 1 eluted off when the percentage of CH₃CN was 45˜50%. The fraction obtained in this way was then loaded on a new C18 cartridge, which was desalted with HPLC water and subject to elution with pure MeOH. The eluent was collected and taken to dryness under vacuum to give conjugate 1 as a greenish brown solid in 52% yield (30 mg). ESI-MS m/z [M-2OAc]²⁺ calc. 889.283, obs. 889.284.

Compound 7: Compound 7 was synthesized using a modification of the procedure used to obtain compound 4. Specifically, by using 1,6-hexanediol as a starting material, product 7 was obtained in the form of a colorless oil in 43% yield. ESI-MS m/z [M+Na]⁺ calc. 306.0948, obs. 306.0948. ¹H NMR (CDCl₃, 400 MHz): δ8.81 (d, 1H, J=1.60 Hz); 8.45-8.42 (m, 1H); 8.24-8.22 (d, 2H, J=9.21 Hz); 7.93-7.91 (m, 1H); 7.45 (t, 2H, J=3.20 Hz); 7.05-7.03 (m, 1H); 6.33 (s, 2H); 6.18 (s, 2H); 3.55 (t, 2H, J =6.80 Hz); 3.48-3.45 (m, 4H); 3.22-3.16 (m, 4H); 1.82 (s, 6H); 1.33 (t, 6H, J=6.80 Hz). ¹³C NMR (CDCl₃, 400 MHz): 196.3, 168.5, 162.5, 156.4, 154.0, 151.9, 147.5, 136.3, 132.6, 131.2, 130.6, 128.7, 128.5, 128.2, 128.1, 123.9, 122.9, 118.0, 106.2, 105.2, 96.8, 77.7, 65.2, 59.1, 41.4, 38.5, 16.8, 14.9 ppm.

Compound 8: Compound 8 was synthesized using a modification of the procedure used to obtain compound 5. Using 3 and 7 as precursors, product 8 was obtained in the form of a green sticky solid in 45% yield. ESI-MS m/z [M-2OAc-H]⁺ calc. 1474.6, obs. 1474.5.

Compound 9: Compound 9 was synthesized using a modification of the procedure used to obtain compound 6. Using 8 and doxorubicin-HCl as precursors, product 9 was obtained as a greenish brown solid in 31% yield. ESI-MS m/z [M-2OAc-H+Na]²⁺ calc. 1033.4, obs. 1033.4.

Conjugate 10: Conjugate 10 was synthesized using a modification of the procedure used to obtain compound 1. Starting from 9, product 10 was obtained in the form of a greenish brown solid in 35% yield. ESI-MS m/z [M-2OAc]²⁺ calc. 871.3270, obs. 871.3281.

### Example 3: Methods and Materials

### 1. Synthetic Materials and Methods

All reagents were purchased from Fisher Scientific, Aldrich, or TCI and used without further purification. All solvents were analytical or HPLC grade. Deionized water was used unless otherwise indicated. Organic solvents were purified using a solvent purifier system (Vacuum Atmospheres) unless otherwise indicated. Dichloromethane was freshly distilled after being dried over CaH₂ under argon. Reverse-phase HPLC experiments were conducted using a Shimadzu HPLC (Shimadzu LC 6AD) with a Thermo Scientific Acclain™ 120 C18 (3 μm, 120 Å, 2.1×150 mm), Shim-pack GIS (5 μm ODS, 250×4.6 mm id) column for analytical studies. The flow rate for analytical HPLC studies was 1.0 mL/min. For the mobile phase, Buffer A (water containing 0.1% v/v acetic acid) and Buffer B (acetonitrile containing 0.1% v/v acetic acid) were used to provide the solvent gradient. Waters Sep-Pak® Vac 35 cc (10 g) t-C18 cartridges were used for preparative work. Acetonitrile and an ammonium acetate buffer were used as the eluent. The ammonium acetate buffer in question was prepared from 32 g of ammonium acetate and 40 mL of acetic acid dissolved in distilled water (total volume, 4 L). Mass spectrometric analyses were carried out in the University of Texas at Austin Mass Spectrometry Facility. Low-resolution and high-resolution electrospray mass spectrometric (ESI-MS) analyses were carried out using a Thermo Finnigan LTQ instrument and a Qq-FTICR (7 Telsa) instrument, respectively.

### 2. UV/Vis and Fluorescence Spectroscopic Methods

All organic solvents used for spectroscopic analyses were HPLC grade and free of fluorescent impurities. Stock solutions of conjugates 1 and 10 were prepared in phosphate buffered saline (PBS; 10 mM, pH 7.4). Fluorescent and UV/Vis absorption spectra were recorded on Shimadzu RF-5301PC and S-3100 spectrophotometers, respectively. Excitation was carried out at 500 nm with both excitation and emission slits widths being set at 3 nm.

### 3. Liposomal Formulations of Conjugates 1 and 10

Lipid-based nanoparticles loaded with 1 were prepared via the thin film hydration method. The lipid compositions consisted of 1, polyethylene glycol (PEG)-cholesterol (NANOCS, NY, USA), 1,2-dioleoyl-sn-glycero-3-phosphoethanolamine (DOPE, Avanti Polar Lipids, AL, USA), 1,2-dioleoyl-sn-glycero-3-phosphoethanolamine-N-[methoxy(polyethylene glycol)-2000] (mPEG-DSPE, Avanti Polar Lipids, USA), and 1,2-distearoyl-sn-glycero-3-phosphoethanolamine-N-[folate(polyethylene glycol)-2000] (folate-PEG-DSPE, Avanti Polar Lipids, USA) at a molar ratio of 4:1:3:1.96:0.04, respectively, for FL-1. Similarly, 10, PEG-Cholesterol, DOPE, mPEG-DSPE, and folate-PEG-DSPE at a molar ratio of 4:1:3:1.96:0.04, respectively, were used to prepare FL-10. Finally, 1, PEG-Cholesterol, DOPE, mPEG-DSPE at a molar ratio of 4:1:3:2, respectively, were used to prepare L-1. In each case, the lipids were completely dissolved in a mixture of chloroform and methanol, and evaporated to form a thin film in a glass tube. The thin film was then hydrated with 10 mM HEPES buffer to generate a final concentration of 4 mM in 1 or 10. After vortexing and sonicating (each for 10 min), the resulting formulations, namely FL-1, FL-10, or L-1, were stored at −4° C. The size and zeta-potential of the liposomal nanoparticles obtained in this way were measured by dynamic light scattering (DLS, Zetasizer Nano, Malvern Instruments, UK).

### 4. MR Contrast Characteristics of FL-1

To evaluate its MRI contrast capability, the T₁ relaxivities of FL-1 at various concentrations in HEPES buffer solution were measured at 60 MHz (0.47 T) and 200 MHz (4.7 T) on a Bruker Minispec system (Bruker, Germany) and an MRI (Biospec 47/40, Bruker, Germany) system, respectively. MRI phantom images were acquired using a 4.7 T MRI instrument (Biospec 47/40, Bruker, Germany). The following scanning parameters were used: MSME (Multi-slice multi-echo) pulse sequence, TE/TR=9.4/350 ms, matrix size=192×192, FOV=2×6 cm, slice thickness=1 mm. A linear fitting of the measured relaxation rates (R₁=1/T₁, s⁻¹) vs. the Gd³⁺ concentration (mM) allowed the relaxivity value (r₁) to be determined.

### 5. Cell Culture and Cellular Uptake Studies

Human cervix carcinoma KB, mouse colon carcinoma CT26, mouse fibroblast NIH3T3 cells, and human hepatocarcinoma HepG2 cells were cultured in 5% CO₂ at 37° C. in Dulbecco's modified Eagle's medium (DMEM, Invitrogen-Gibco, Carlsbad, Calif., USA) and minimum essential medium (MEM, Invitrogen-Gibco), respectively, supplemented with 1× Antibiotic-Antimycotic (Invitrogen-Gibco, Carlsbad, Calif., USA) and 10% fetal bovine serum (Invitrogen-Gibco). For confocal fluorescence cell imaging, the cells were plated on 8-well μ-slides (Ibidi, Munich, Germany) for 12 h. After treatment with 4 μM of either FL-1 or L-1 for 1 h, the cells were washed 3 times and fixed with Cytofix fixation buffer (BD Biosciences, San Jose, Calif., USA) for 10 min at room temperature. Fluorescence cellular images were acquired by using a laser scanning confocal microscope (LSM 710, Carl Zeiss, Germany) after counterstaining with Hoechst 33342 ((Molecular Probes, Eugene, Oreg., USA). For the quantitative analysis of FL-1 uptake into cells, flow cytometry was performed on the same four cell lines used in the imaging experiments. For these experiments, the cells were plated on 12-well culture plates at a density of 2×10⁵ cells per well and incubated for 12 h. FL-1 (4 μM) was added to each well. After 1 h, the fixed cells were analyzed using flow cytometry (Attune acoustic focusing cytometer, Applied Biosystems, USA).

### 6. Evaluation of Anti-Cancer Effects

The anti-proliferative activity of FL-1 and FL-10 was determined using a 3-(4,5-dimethylthiazole-2-yl)-2,5-diphenyl tetrazolium bromide (MTT; Roche Diagnostics GmbH, Mannheim, Germany) assay. KB, CT26, HepG2, and NIH3T3 cells cultured in 96-well plates were incubated for 24 h at a density of 4×10³ cells/well. After replacing with fresh culture medium, the cells were treated with various concentrations of FL-1 or FL-10, followed by additional incubation for 48 h. Then, the cells were added to the MTT solution, allowed to stand for 4 h, and treated with a solubilization buffer (Roche Diagnostics). Absorbance was read at 570 nm using a microplate reader (VersaMax; Molecular Devices, CA, USA).

### 7. Fluorescence Imaging and T₁-Weighted MR Contrast Enhancement of Cell Pellets Obtained After Treatment of Cells with 1

To produce cell pellets for imaging, KB cells were treated for 12 h with various concentrations of FL-1. Fluorescence images were obtained using a Maestro In Vivo Imaging System (CRI, Inc., Woburn, Mass., USA) and T₁-weighted MR images were obtained using the multi-slice multi-echo (MSME) sequence of the 4.7 T MRI instrument. The following acquisition parameters for the MRI studies were used: field of view (FOV)=6×3 cm², matrix size=192×192, NEX=4, slice thickness=1 mm, echo time (TE)=10 ms, repetition time (TR)=400 ms.

### 8. In Vivo Imaging and Evaluation of Therapeutic Efficacy in Tumor Mouse Models

All animal experiments followed the guidelines of the U.S. National Institutes of Health and the recommendations of the committee on animal research at the Korea Basic Science Institute. The protocol was approved by the local institutional review committee on animal care (KBSI-AEC1009). To establish an orthotopic mouse model of liver metastasis from colon cancer, CT26 cells (2.5×10⁵) were injected intrasplenically to 6-week-old male Balb/C mice (Orient Bio, Seungnam, Gyeonggi-do, South Korea) under isoflurane anesthesia. For the evaluation of early diagnosis by MRI, FL-TssD or saline as a control was injected into the tail vein at a single dose of 20 mg/kg 3 days post inoculation. The liver tissue was then examined via T₁-weighted MRI. T₁-weighted MR images were obtained using a multi-slice multi-echo (MSME) sequence, TE=10 ms, TR=300 ms, slice thickness=1 mm, field of view (FOV)=3.5×3.5 cm², NEX=4, matrix size=256×256. A comparison of MR contrast enhancement between normal and tumor areas in the liver tissue was determined by a signal-to-noise ratio (SNR) analysis. For the non-invasive monitoring of the therapeutic response using the metastatic orthotopic models, mice (n=8) were intravenously injected with 2.5 mg/kg of FL-1 and saline as a control, four times at 2-day intervals. The efficacy of the anti-cancer activity was evaluated by T₂-weighted MR imaging on days 7, 14, 21, following administration of FL-1. The T₂-weighted MR imaging parameters used were as follows: Turbo-RARE (rapid acquisition with a relaxation enhancement), TE=36 msec, TR=3500 msec, slice thickness=1 mm, FOV=3.5×3.5 cm², NEX=4, matrix size=256×256. In a second in vivo experiment, FL-1 was administered intravenously to subcutaneous xenograft tumor mice induced by KB cells (1×10⁶) using the same dose and treatment times as above. Tumor-targeted visualization over the whole body was effected in vivo using fluorescence imaging or MRI (SNR analysis; MSME sequence, TE=10 ms, TR=350 ms, slice thickness=1 mm, FOV=3.5×3.5 cm², NEX=4, matrix size=256×256). Efficacy was evaluated by caliper-based monitoring of tumor growth as a function of time.

### Example 4: Conjugates with Hydrazone Linker

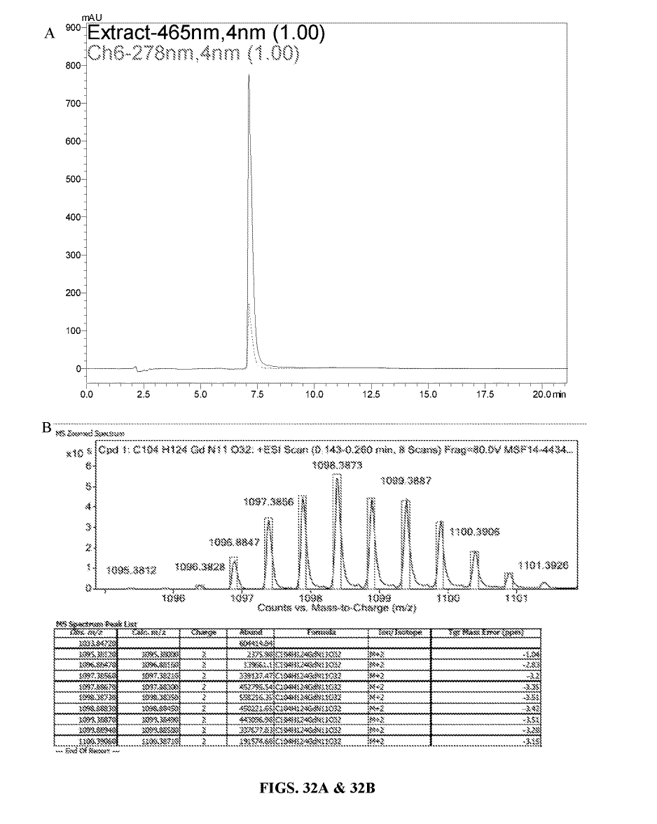

Conjugate 11 was prepared as outlined in Scheme 4. The starting MGd was prepared in accord with published procedures (Sessler et al., 1999; Wei et al., 2005). The MGd was treated with 4-nitrophenyl chloroformate in the presence of N,N-diisopropyethylamine (DIPEA) as a base in dry dichloromethane (DCM) to give 3. Compound 2 was then obtained by reacting 3 with hydrazine. Imination of 2 with doxorubicin in the presence of a catalytic amount of trifluoroacetic acid (TFA) in MeOH gave 1 in 51% yield. As expected for a system containing a paramagnetic Gd³⁺ center, the ¹H NMR spectrum was characterized by substantial peak broadening. Therefore, as true for the previously reported platinum-based Gd³⁺ texaphyrin conjugates, the purity and chemical structure of 11, 12, and 13 were confirmed by HPLC and ESI mass spectrometry (spectra described in FIGS. 32A & 32B).

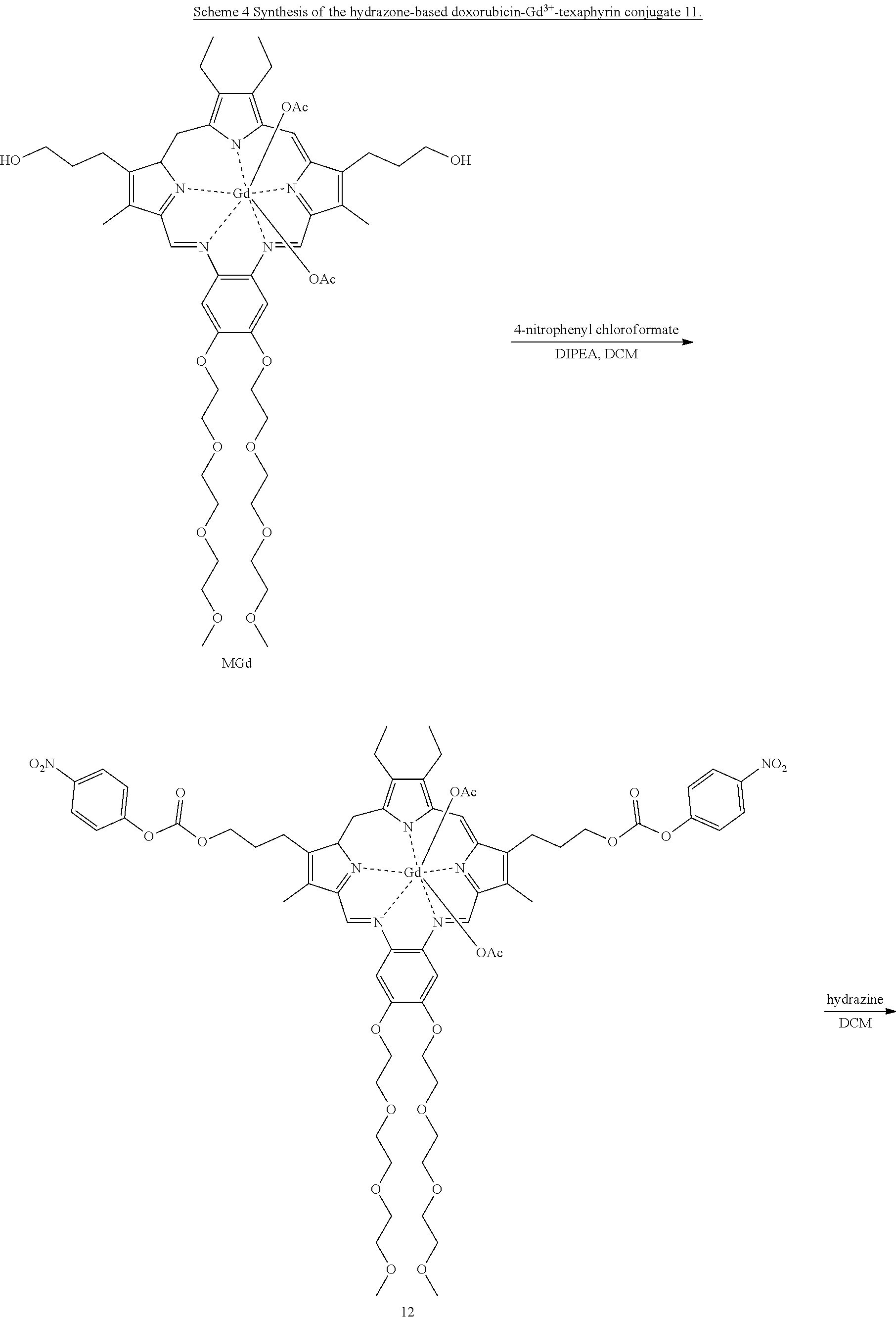

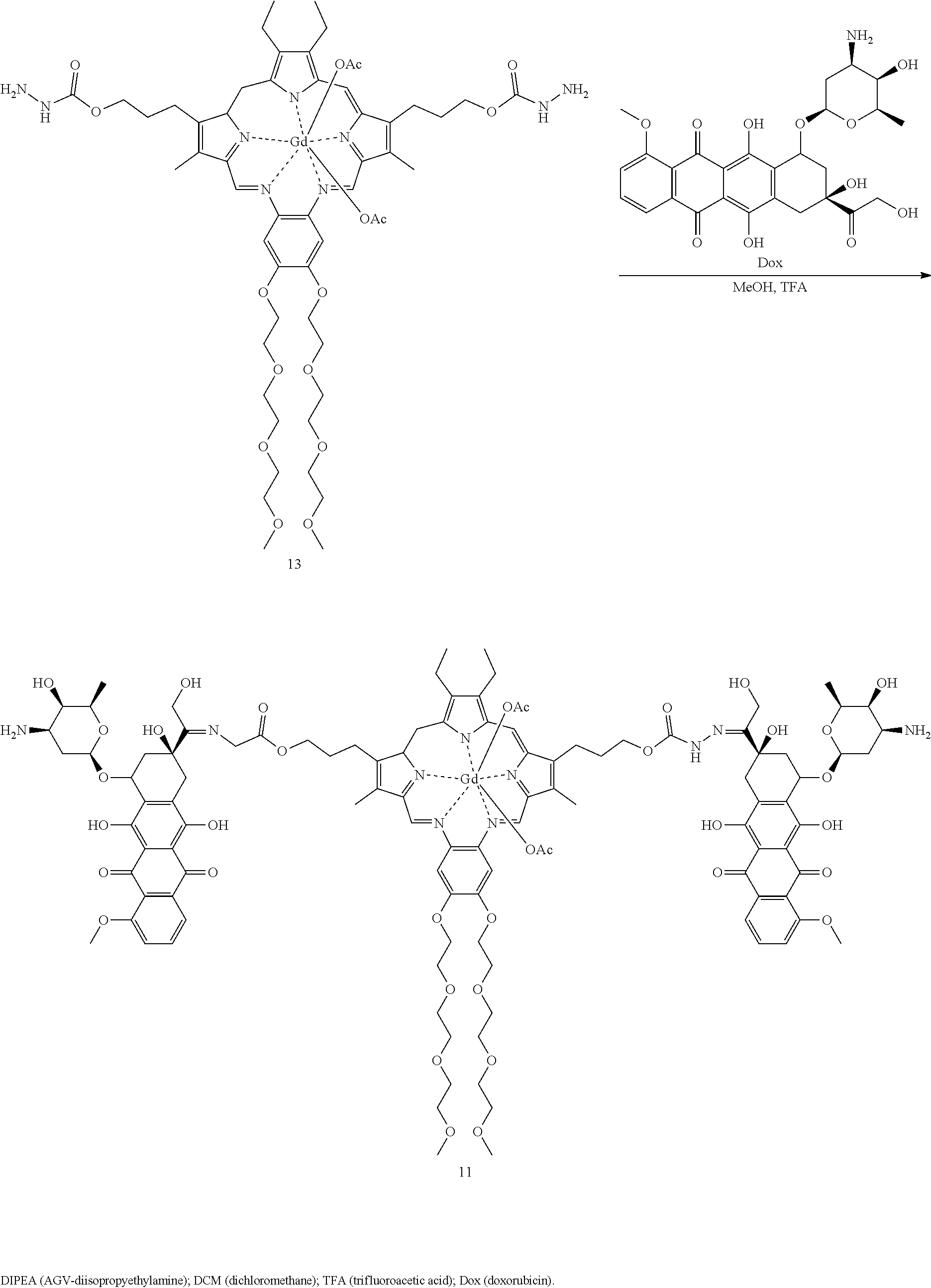

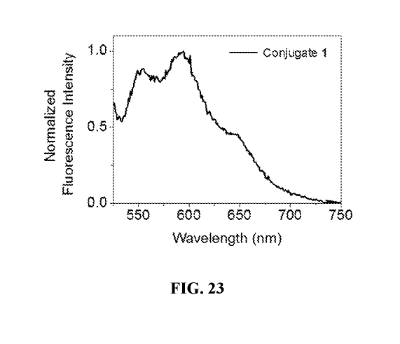

The potential for conjugate 11 to provide a turn-on fluorescent signal as Dox is released was assessed first by comparing the UV/Vis absorption and fluorescence spectroscopic features of the intact conjugate and free Dox. As seen by an inspection of FIG. 22A and FIG. 22B, free Dox is characterized by a broad absorption and a strong red emission features at around 500 and 600 nm, respectively, in PBS buffer (pH 7.4). In contrast, the UV/Vis absorption spectrum of conjugate 11 contains two strong absorption bands at 477 and 750 nm as well as a broad absorption band at 550 nm, characteristic of Gd³⁺ texaphyrin moiety. Absorption band for the chemically linked Dox is not clearly observed, presumably due to a high absorption coefficient of the Gd³⁺ texaphyrin moiety. Upon excitation of 500 nm, 11 shows a very weak emission feature, ascribed to the Dox subunit, is seen in the 550-700 nm spectral region (FIG. 22B and FIG. 23). The low fluorescence intensity is ascribed to quenching of the Dox excited state by the paramagnetic Gd³⁺-texaphyrin moiety in 11.

To gain insight into whether the key hydrazone linkages present in 11 is cleaved under acidic conditions, time-dependent changes in the fluorescence intensity were monitored at two different pH values. These studies revealed that in acetate buffer (pH 5.0), the fluorescence intensity of 11 at 593 nm gradually increases as a function of time (FIGS. 22C & 22D). In contrast, at pH 7.4 (phosphate buffered saline; PBS) the extent of fluorescence intensity increase was substantially lower under otherwise identical conditions (FIG. 22D). These results were taken as support for the suggestion that the hydrazone bond of 11 would undergo cleavage to release free Dox within the acidic endosomal compartments of cancer cells.

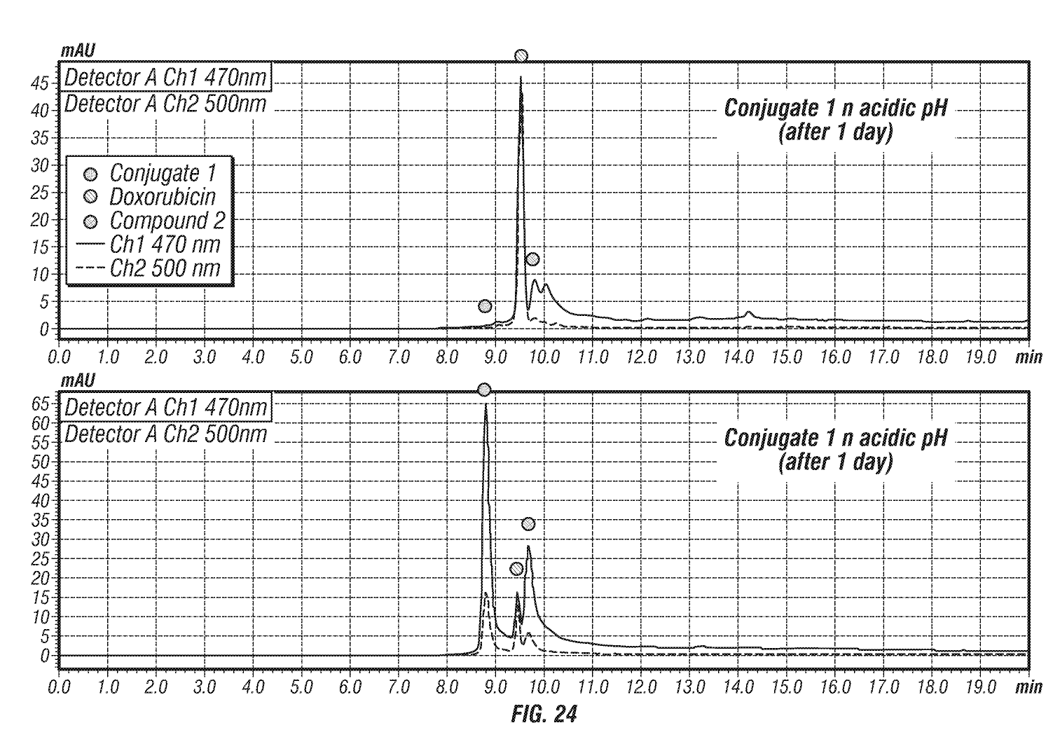

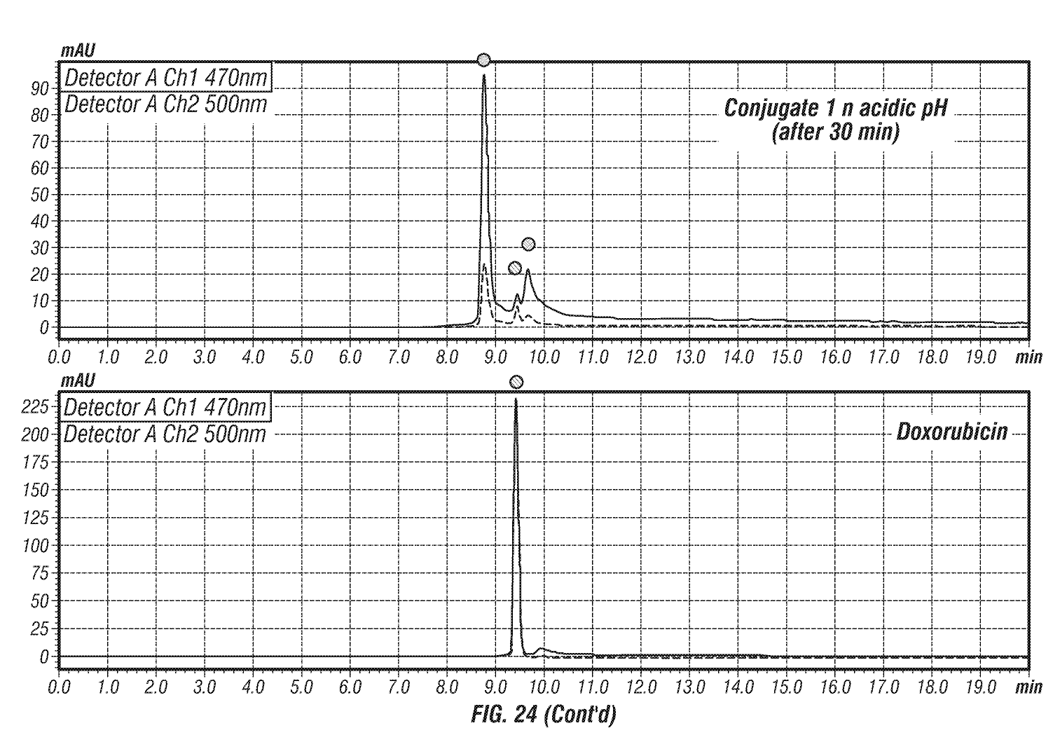

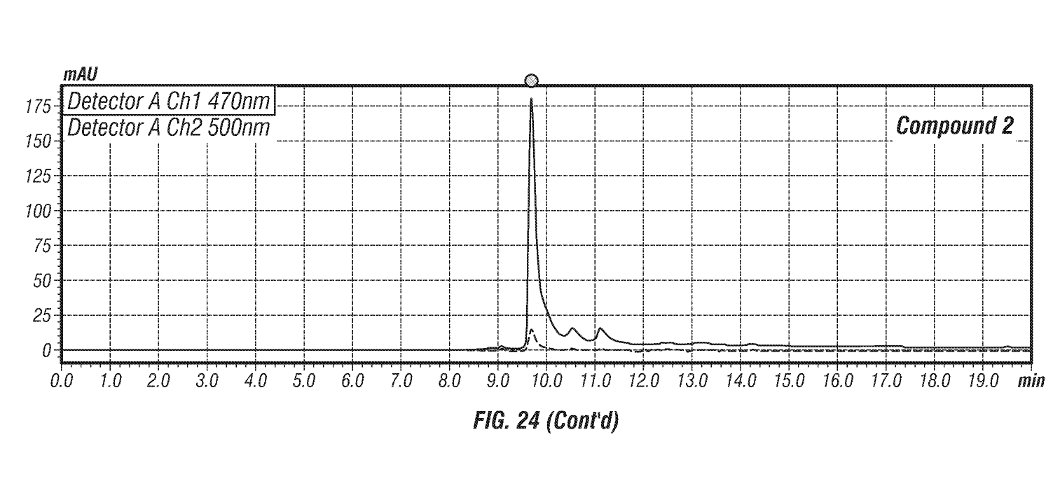

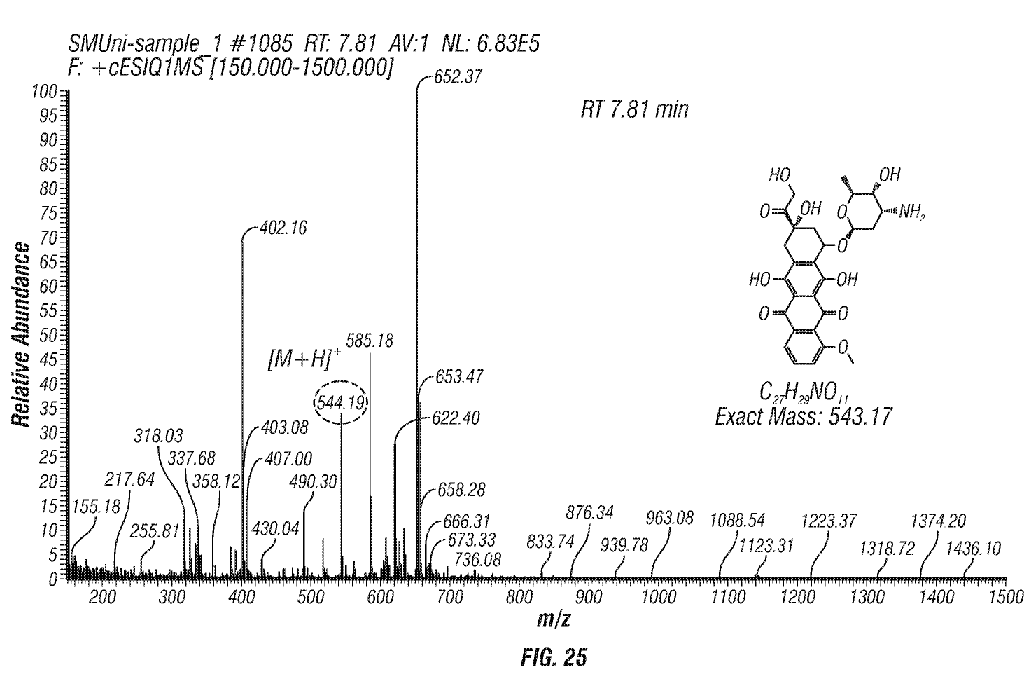

Further evidence that the hydrazone bond present in 11 would undergo cleavage at relatively low came from a time-dependent reverse phase-HPLC analysis. Specifically, it was found that incubating 11 in an acetate buffer at pH 5.0 led to Dox release and that extent of release increased with time (FIG. 24). After 1 day of incubation, essentially complete conversion of conjugate 11 to free Dox and the functionalized texaphyrin derivative 13 was observed. The chemical identity of the free Dox produced in this way was confirmed by ESI-Mass spectrometry (FIG. 25).

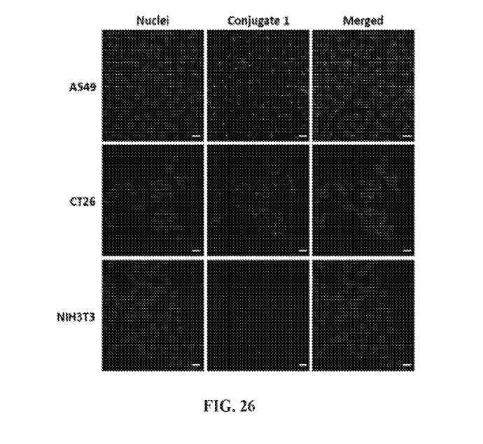

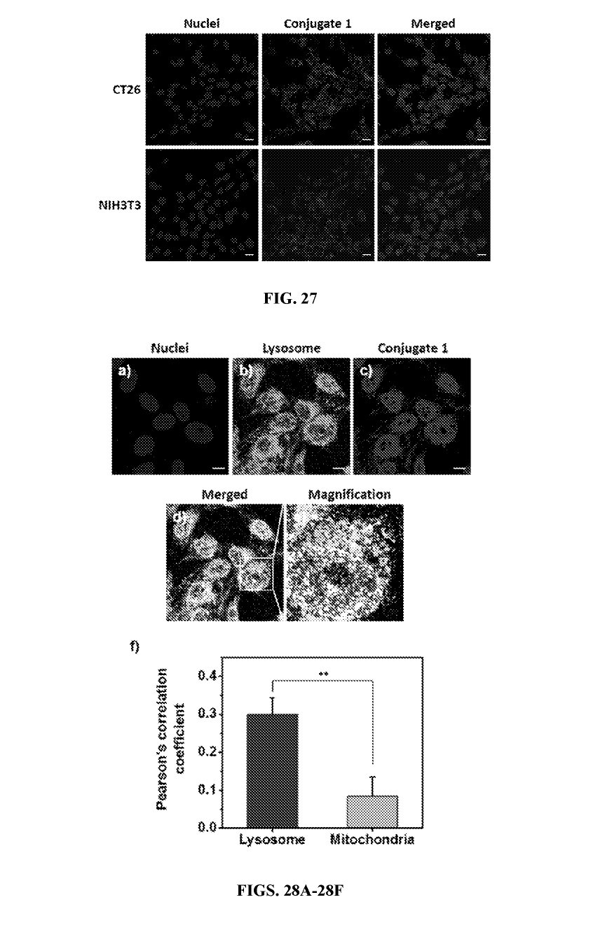

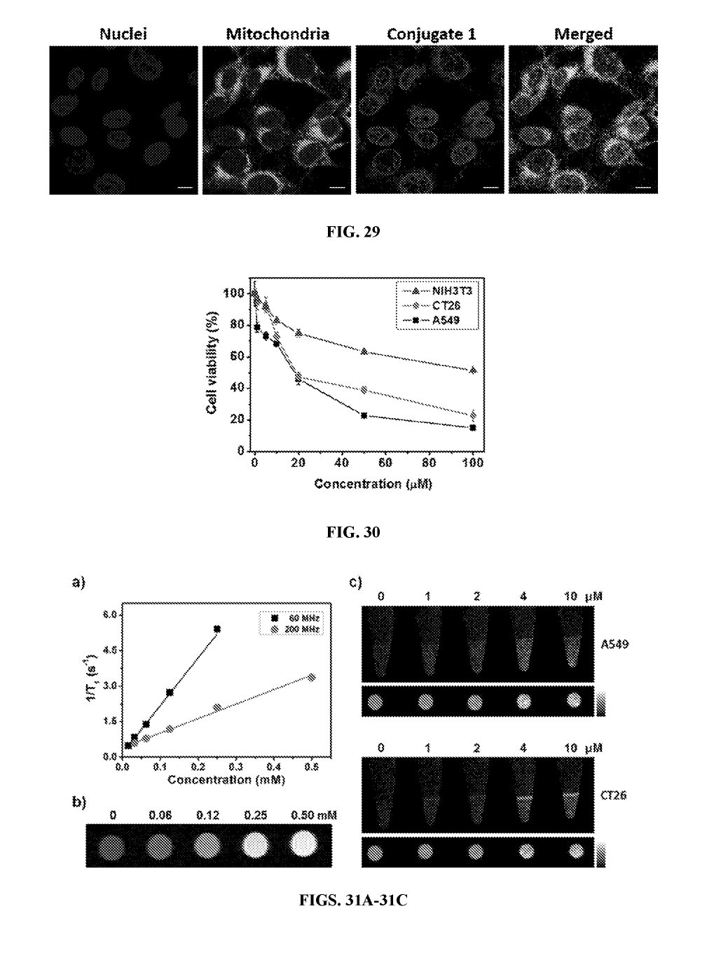

Intracellular uptake of conjugate 11 was evaluated by comparing the fluorescence of free Dox released from conjugate 11 in two cancer cell lines (A549 and CT26) and a non-cancerous fibroblast cell line (NIH3T3). After incubating with 11 for 1 h, strong fluorescence images were seen in the case of both the A549 and CT26 cancer cell lines, whereas a very weak fluorescence signal was seen in the corresponding NIH3T3 cell studies (FIG. 26). On the other hand, after a 12 incubation time period no significant difference in the fluorescence signals for the CT26 and NIH3T3 cell lines was observed (FIG. 27). Taken in concert, these findings provide support for the notion that as compared to the non-cancerous control (NIH3T3 cells), conjugate 11 is taken up relatively quickly by the test cancer cells (A549 and CT26) and that once taken up 11 undergoes an acid-mediated hydrazone cleavage to release free Dox.

It is generally accepted that doxorubicin enters the nucleus, interacts with topoisomerase II, and induces cell death (Gewirtz, 1999). It was then determined if prodrug 11 would provide of source of Dox within nuclei. Co-localization experiments were thus performed in CT26 cells using fluorescent trackers for nuclei, lysosomes, and mitochondria, respectively. Here, CT26 cells were incubated with 10 μM of 11 for 12 h. They were then stained with LysoTracker (lysosome), MitoTracker (mitochondria), and Hoechst (nuclei), respectively. As can be seen from an inspection of FIG. 28, a strong red fluorescence signal readily assigned to the Dox released from conjugate 11 was found inside the nuclei. The nucleus localization inferred on this basis was supported by the merged images showing co-localization of the Hoechst- and Dox-derived fluorescent signals (FIG. 28D). In addition, the red Dox fluorescence was also seen to overlap with the green fluorescence of lysosomes, leading us to propose that hydrazone cleavage and Dox release occurs predominantly in the relatively acidic lysosomes, rather than, e.g., the mitochondria (FIGS. 28E, 28F, & 29). Once released the free Dox translocates to the nuclei where it exerts its established anticancer effect.

The extent to which conjugate 11 would mediate an anticancer effect was assessed in vitro using a standard MTT assay. As shown in FIG. 30, when the A549 and CT26 cancerous cells were treated with 1, dose-dependent decreases in cell viability were observed. However, in the case of NIH3T3 cells, a non-cancerous fibroblast cell line, substantial survival was seen even when incubated with a high concentration of 11 (˜100 μM) under the same conditions. At low concentrations (≤10 μM) the different cytotoxicity profiles seen for these two cell lines is consistent with the relative cellular uptake efficiency seen for 11 in the A549, CT26, and NIH3T3 cells as revealed in FIG. 26. On this basis, it was concluded that free Dox is released from conjugate 11 more effectively in the A549 and CT26 cancerous cells than in the non-cancerous NIH3T3 fibroblast cells.

A potential beneficial feature of conjugate 11 is that it contains a paramagnetic Gd³⁺ center. It was thus expected to allow for facilitated MR imaging. Its potential in this regard was first probed by measuring longitudinal relaxation rates as a function of concentration and field strength. On the basis of the plot displayed in FIG. 31A, the T₁ relaxivities of 11 in PBS were calculated to be 20.1±0.4 and 6.1±0.2 mM⁻¹s⁻¹ at 60 and 200 MHz, respectively. As true for MGd itself, these values are significantly higher than those for commercial Gd³⁺-based T₁ contrast agents, such as Magnevist® (Bayer Healthcare, USA) and Omniscan® (Amersham, USA) (Bhuniya et al., 2011). MR phantom images were then collected at 200 MHz (4.7 T MR scanner) as the concentration of 11 in PBS was increased. These studies revealed a concentration dependent increase in contrast (FIG. 31B). Finally, T₁-contrast MR imaging of A549 and CT26 cancer cells was carried out using 11. Here, analysis of the cell pellet phantom revealed a T₁ contrast enhancement that becomes saturated upon treatment of either cell line with 4 μM of 11 (FIG. 31C). The relatively low concentration of 11 needed to reach saturation is consistent with the high T₁ relaxivity displayed by 11. This stands in contrast to the fluorescence signal provided by the free Dox, which permits cellular and subcellular imaging only after the hydrazone linkage undergoes hydrolysis. The ability to detect in a non-invasive fashion both the intact and cleaved forms of conjugate 11 (and its Dox payload) is considered to be a potentially useful feature that may make systems such as those described here useful as theranostics.

### Example 5: Methods and Materials for Conjugates with Hydrazone Linker

### 1. Synthetic Materials and Methods

All reagents were purchased from Fisher Scientific, Aldrich, or TCI and used without further purification. All solvents were analytical or HPLC grade. Deionized water was used unless otherwise indicated. Organic solvents were purified using a solvent purifier system (Vacuum Atmospheres) unless otherwise indicated. Dichloromethane was freshly distilled after being dried over CaH₂ under argon. Reverse-phase HPLC experiments were conducted using a Shimadzu HPLC (Shimadzu LC 6AD) with a Thermo Scientific Acclain™ 120 C18 (3 μm, 120 Å, 2.1×150 mm) Shim-pack GIS (5 μm ODS, 250×4.6 mm id) column for analytical studies and Waters Sep-Pak® Vac 35 cc (10 g) t-C18 Cartridges for preparative work. The flow rates for the analytical studies was 1.0 mL/min. For the mobile phase, Buffer A (water with 0.1% v/v acetic acid) and Buffer B (acetonitrile with 0.1% v/v acetic acid) were used to provide the solvent gradient. Mass spectrometric analyses were carried out in the University of Texas at Austin Mass Spectrometry Facility. Low-resolution and high-resolution electrospray mass spectrometric (ESI-MS) analyses were carried out using a Thermo Finnigan LTQ instrument and a Qq-FTICR (7 Telsa) instrument, respectively.

2. UV/Vis and fluorescence Spectroscopic Methods

All organic solvents used for spectroscopic analyses were HPLC grade free of fluorescent impurities. Stock solutions of conjugate 11 were prepared in DMSO. Phosphate buffered saline (PBS) (20 mM, pH 7.4) and acetate buffer (20 mM, pH 5.0) were prepared in deionized water. All spectra were recorded in this buffer solution containing 1% (v/v) DMSO. The fluorescence and UV/Vis absorption spectra were recorded on Shimadzu RF-5301PC and S-3100 spectrophotometers, respectively. Excitation was carried out at 500 nm with both the excitation and emission slits widths being set at 3 nm.

3. T₁-Weighted MR Contrast Properties of Conjugate 11 in PBS Solution

The T₁ relaxivity of conjugate 11 in aqueous solution was measured so as to evaluate its potential for MR imaging. Conjugate 11 was analyzed at various concentrations in PBS solution at 60 MHz (1.4 T) and 200 MHz (4.7 T) using a Minispec system (Bruker, Germany), and a MRI (Biospec 47/40, Bruker, Germany) system, respectively, and imaged using a 4.7 T MRI instrument (Biospec 47/40). The following scanning parameters were used: MSME (Multi-slice multi-echo) pulse sequence, TE/TR=9.4/350 ms, matrix size=192×192, FOV=2×6 cm, slice thickness=1 mm. Linear fitting of the measured relaxation rates (R₁=1/T₁, s⁻¹) vs. the Gd³⁺ concentration (mM) was used to determine the relaxivity values, r₁.

4. Cell Culture and Fluorescence Imaging

Human lung cancer A549, mouse colon carcinoma CT26, and mouse fibroblast NIH3T3 cells were grown in 5% CO₂ at 37° C. in Dulbecco's modified Eagle's medium (DMEM) supplemented with 1× Antibiotic-Antimycotic (Invitrogen-Gibco, Carlsbad, Calif., USA) and 10% fetal bovine serum (Invitrogen-Gibco). For fluorescence microscope imaging, the cells were seeded on 8-well μ-slides (Ibidi, Munich, Germany) for 24 h and were treated with 1 at a concentration of 4 μM for an additional 1 h or 12 h. The cells were then washed 3 times and fixed in Cytofix fixation buffer (BD Biosciences, San Jose, Calif., USA) for 30 min at 4° C. and counterstained with Hoechst 33342 (Molecular Probes, Eugene, Oreg., USA) at 5 μg/mL. For co-localization studies, CT26 cells were stained with 100 nM LysoTracker Green DND-26 (Molecular Probes) or 50 nM MitoTracker Green FM (Molecular Probes) for 1 h at 37° C. Fluorescence images were obtained using a laser scanning confocal microscope (LSM 710, Carl Zeiss, Germany).

5. Cell Viability Measurement of Cells Treated with Conjugate 11

The cell viability of A549, CT26, and NIH3T3 cells incubated with conjugate 11 was assessed by a 3-(4,5-dimethylthiazole-2-yl)-2,5-diphenyl tetrazolium bromide (MTT; Roche Diagnostics GmbH, Mannheim, Germany) assay. The cells were seeded in 96-well plates for 24 h at a density of 2×10³ cells/well and then replaced with fresh culture medium. The cells were treated with various concentrations of 11, and incubated for 48 h. MTT in a solubilizing buffer as purchased commercially (Roche Diagnostics) added to each well and allowed to incubate for 4 h. Absorbance was measured at 570 nm using a microplate reader (VersaMax; Molecular Devices, CA, USA).

6. T1-Weighted MR Contrast Enhancement of Cells Treated with Conjugate 11

T₁-weighted spin-echo images of cell phantoms were obtained on a 4.7 T MRI instrument using A549 and CT26 cells labeled with different concentrations of 1. The following acquisition parameters were used: Field of view (FOV)=5.0×2.5 cm² and 4×5 cm², matrix size=192×192, slice thickness=1 mm, echo time (TE)=9.4 ms, repetition time (TR)=350 ms.

### Example 6: Synthetic Characterization of Conjugates with Hydrazone Linker

The starting texaphyrin (MGd) was prepared using literature procedures (Sessler et al., 1999; Wei et al., 2005).

Synthesis of compound 12: MGd (300 mg, 0.26 mmol) and 4-nitrophenyl chloroformate (525 mg, 2.61 mmol) were dissolved in distilled dichloromethane (DCM) (60 mL). N,N-diisopropylethylamine (DIPEA) (930 μL, 5.22 mmol) was slowly added to the reaction mixture, which was then stirred for 4 h at room temperature. The progress of the reaction was monitored by HPLC. When the starting material (MGd) was no longer present (as inferred from this analysis), the solvent was evaporated off under reduced pressure and the resulting precipitates were collected with the aid of diethyl ether washings. The product, 12, was obtained as a dark green solid in 87% yield (330 mg). ESI-MS m/z [M-2OAc]²⁺ calc. 680.20900, obs. 680.21060.

Synthesis of compound 13: To a solution of 12 (200 mg, 0.13 mmol) in acetonitrile (CH₃CN) (10 mL), hydrazine monohydrate (68 μL, 1.35 mmol) was slowly added. The reaction mixture was then stirred for 2 h at room temperature. The progress of the reaction was monitored by HPLC. When the starting material 12 was no longer present (as inferred from this analysis), the solvent was evaporated off under reduced pressure and the resulting residue was purified using Waters Sep-Pak® Vac 35 cc (10 g) tC18 Cartridges. For this purification, 50 mL of a 0.1 M ammonium acetate buffer solution (4 L of distilled water containing 32 g of ammonium acetate and 40 mL of acetic acid) was added and the resulting solution was loaded onto the C18 cartridge and subject to elution with an increasing gradient of CH₃CN (10˜90%) in an ammonium acetate buffer. The product, 13, eluted off when the eluent contained 25˜30% of CH₃CN. The fraction obtained in this way was then loaded on a new C18 cartridge, desalted with HPLC-grade deionized water, and eluted off using pure methanol (MeOH). The volatiles were removed under vacuum to give the product 13 as a dark green solid in 30% yield (50 mg). ESI-MS m/z [M-2OAc]²⁺ calc. 573.21930, obs. 573.22090.

Synthesis of compound 11: Doxorubicin.HCl (20 mg, 0.016 mmol) and 13 (28 mg, 0.047 mmol) were dissolved in anhydrous methanol (MeOH) (10 mL) and the mixture was treated with trifluoroacetic acid (TFA) (5 μL). After stirring for 2 days at room temperature in the dark, all volatiles were evaporated off under vacuum. The residue obtained in this way was purified using Waters Sep-Pak® Vac 35 cc (10 g) tC18 Cartridges. Here, 50 mL of a 0.1 M ammonium acetate buffer solution (4 L of distilled water containing 32 g of ammonium acetate and 40 mL of acetic acid) was added and the resulting solution was loaded onto the C18 cartridge and subject to elution with increasing gradient of CH₃CN (10˜90%) in an ammonium acetate buffer. The product, 11, eluted off when the eluent contained 20˜30% of CH3CN. The fraction obtained in this way was then loaded on a new C18 cartridge, desalted with HPLC-grade deionized water, and eluted off using pure methanol (MeOH). The solvent was removed under vacuum to give product 11 as a brown solid in 51% yield (19 mg). ESI-MS m/z [M−2OAc]²⁺ calc. 1,098.38850, obs. 1,098.38730.

All of the compositions and methods disclosed and claimed herein can be made and executed without undue experimentation in light of the present disclosure. While the compositions and methods of this disclosure have been described in terms of certain embodiments, it will be apparent to those of skill in the art that variations may be applied to the compositions and methods and in the steps or in the sequence of steps of the methods described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

U.S. Pat. No. 4,935,498

U.S. Pat. No. 5,252,270

U.S. Pat. No. 5,272,142

U.S. Pat. No. 5,292,414

U.S. Pat. No. 5,369,101

U.S. Pat. No. 5,432,171

U.S. Pat. No. 5,439,570

U.S. Pat. No. 5,504,205

U.S. Pat. No. 5,569,759

U.S. Pat. No. 5,583,220

U.S. Pat. No. 5,587,463

U.S. Pat. No. 5,591,422

U.S. Pat. No. 5,633,354

U.S. Pat. No. 5,776,925

U.S. Pat. No. 5,955,586

U.S. Pat. No. 5,994,535

U.S. Pat. No. 6,207,660

U.S. Pat. No. 7,112,671

U.S. Pat. No. 8,410,263

Bardhan et al., Acc. Chem. Res., 44, 936-946, 2011.

### Bhuniya et al., Biomaterials, 32, 6533, 2011.

### Cera and Palumbo, Anti-cancer Drug Design, 5(3):265-271, 1990.

### Fritzsche, et al., Biochemistry, 26(7):1996-2000, 1987.

Gewirtz, Biochem. Pharmacol., 57, 727, 1999.

### Handbook of Pharmaceutical Salts: Properties, and Use, Stahl and Wermuth Eds., Verlag Helvetica Chimica Acta, 2002.

Hannah, et al., Org. Lett., 3(24):3911-3914, 2001.

### Kizek, et al., Pharmacology & Therapeutics, 133(1):26-39, 2012.

### Kokudo and Ishizawa, Liver Cancer, 1, 15-21, 2012.

Kumar et al., Chem. Soc. Rev., 44, 6670-6683, 2015.
Lee et al., Acc. Chem. Res., 48, 2935-2946, 2015.
Lee, et al., J. Am. Chem. Soc., 134, 12668-12674, 2012.

### March's Advanced Organic Chemistry: Reactions, Mechanisms, and Structure,2013.

Mehta et al., Int. J. Radiat. Oncol. Biol. Phys., 73, 1069-1076, 2009.

### Namasivayam et al., Cancer Imaging, 7, 2-9, 2007.

### Olano, et al., Marine Drugs, 7(2):210-248, 2009.

Patel et al., Clin. Cancer Res., 14, 4869-4876, 2008.

### Practical Process Research & Development—A Guide for Organic Chemists, 2012.

Preihs et al., Inorg. Chem., 52, 12184-12192, 2013.

### Rabbani, et al., Bioessays, 27(1):50-56, 2005.

### Schima et al., Cancer Imaging, 5A, S149-156, 2005.

### Semelka et al., Radiology, 213, 86-91, 1999.

Sessler et al., J. Am. Chem. Soc., 115, 10368-10369, 1993.
Sessler et al., J. Phys. Chem. A, 103, 787, 1999.
Shanmugam et al., Chem. Soc. Rev., 43, 6254-6287, 2014.

### Shimada et al., World Journal of Surgical Oncology, 13, 198, 2015.

Shimanovich, et al., J. Am. Chem. Soc., 123:3613-3614, 2001.
Vivero-Escoto et al., Chem. Soc. Rev., 41, 2673-2685, 2012.
Wei et al., Org. Biomol. Chem., 3, 3290-3296, 2005.
Young et al., Proc. Natl. Acad. Sci. USA, 93, 6610-6615, 1996.

What is claimed is:

1. A compound of the formula: 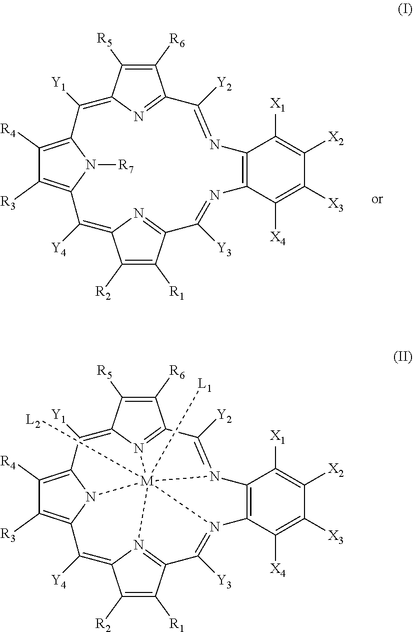
wherein:
Y₁-Y₄ are each independently selected from: hydrogen, amino, cyano, halo, hydroxy, or hydroxyamino,
alkyl_{(C≤12)}, cycloalkyl_{(C≤12)}, alkenyl_{(C≤12)}, cycloalkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, alkoxy_{(C≤12)}, acyloxy_{(C≤i}2), aryloxy_{(C≤12)}, heteroaryloxy_{(C≤12)}, heterocycloalkoxy_{(C≤i}2), amido_{(C≤12)}, alkylamino_{(C≤12)}, dialkylamino_{(C≤12)}, alkylthio_{(C≤i}2), arylthio_{(C≤i}2), alkylsulfinyl_{(C≤12)}, arylsulfinyl_{(C≤12)}, alkylsulfonyl_{(C≤12)}, arylsulfonyl_{(C≤12)}, or a substituted version of any of these groups; or
R₁-R₆ are each independently selected from: hydrogen, amino, cyano, halo, hydroxy, hydroxyamino, or nitro,
alkyl_{(C≤12)}, cycloalkyl_{(C≤12)}, alkenyl_{(C≤12)}, cycloalkenyl_{(C≤12)}, alkynyl(ci2), aryl(ci2), aralkyl_{(C≤12)}, heteroaryl_{(C≤12)}, heterocycloalkyl_{(C≤12)}, acyl_{(C≤12)}, alkoxy_{(C≤12)}, acyloxy_{(C≤2)}, aryloxy_{(C≤12)}, heteroaryloxy_{(C≤12)}, heterocycloalkoxy_{(C≤12)}, amido_{(C≤12)}, alkylamino_{(C≤12)}, dialkylamino_{(C≤12)}, or a substituted version of any of these groups; or
a PEG moiety wherein the PEG moiety is of the formula: —(OCH₂CH₂)ₚOR₉; wherein:
p is 1-20; and
R₉ is hydrogen, alkyl_{(C≤8)}, or substituted alkyl_{(C≤8)}; or
an antitumor antibiotic linked through a cleavable covalent linker;
R₇ is hydrogen,
alkyl_{(C≤8)}, cycloalkyl_{(C≤8)}, alkenyl_{(C≤8)}, cycloalkenyl_{(C≤8)}, alkynyl_{(C≤8)}, alkoxy_{(C≤8)}, or a substituted version of any of these groups, or an amino protecting group;
X₁-X₄ are each independently selected from: hydrogen, amino, cyano, halo, hydroxy, hydroxyamino, or nitro,
alkyl_{(C≤12)}, cycloalkyl_{(C≤12)}, alkenyl_{(C≤12)}, cycloalkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, alkoxy_{(C≤12)}, acyloxy_{(C≤12)}, aryloxy_{(C≤12)}, heteroaryloxy_{(C≤12)}, heterocycloalkoxy_{(C≤12)}, amido_{(C≤12)}, alkylamino_{(C≤12)}, dialkylamino_{(C≤12)}, or a substituted version of any of these groups; or
a PEG moiety wherein the PEG moiety is of the formula: —(OCH₂CH₂)nORs; wherein:
n is 1-20; and
R₈ is hydrogen, alkyl_{(C≤8)}, or substituted alkyl_{(C≤8)};
L₁ and L₂ are each independently absent, a neutral ligand, or an anionic ligand; and
M is a metal ion;
or a pharmaceutically acceptable salt thereof.

2. The compound of claim 1 further defined as: 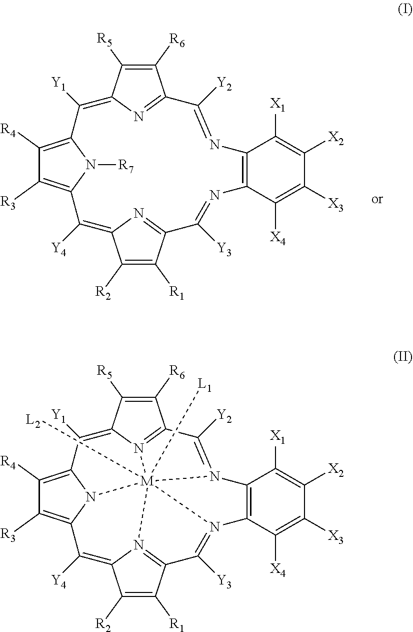
wherein:
Y₁ and Y₄ are each independently selected from: hydrogen, amino, cyano, halo, hydroxy, or hydroxyamino,
alkyl_{(C≤12)}, cycloalkyl_{(C≤12)}, alkenyl_{(C≤12)}, cycloalkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, alkoxy_{(C≤12)}, acyloxy_{(C≤≤12}), aryloxy_{(C≤12)}, heteroaryloxy_{(C≤12)}, heterocycloalkoxy_{(C≤12)}, amido_{(C≤12)}, alkylamino_{(C≤12)}, dialkylamino_{(C≤12)}, alkylthio_{(C≤12)}, arylthio_{(C≤12)}, alkylsulfinyl_{(C≤12)}, arylsulfinyl_{(C≤12)}, alkylsulfonyl_{(C≤12)}, arylsulfonyl_{(C≤12)}, or a substituted version of any of these groups;
Y₂ and Y₃ are each independently selected from hydrogen, alkyl_{(C1-6)}, or substituted alkyl_{(C1-6)};
R₁-R₆ are each independently selected from: hydrogen, amino, cyano, halo, hydroxy, hydroxy amino, or nitro,
alkyl_{(C≤12)}, cycloalkyl_{(C≤12)}, alkenyl_{(C≤12)}, cycloalkenyl_{(C≤12)}, alkynyl_{(C≤2)}, aryl_{(C≤12)}, aralkyl_{(C≤12)}, heteroaryl_{(C≤12)}, heterocycloalkyl_{(C≤12)}, acyl_{(C≤12)}, alkoxy_{(C≤12)}, acyloxy_{(C≤12)}, aryloxy_{(C≤12)}, heteroaryloxy_{(C≤12)}, heterocycloalkoxy_{(C≤12)}, amido_{(C≤12)}, alkylamino_{(C≤12)}, dialkylamino_{(C≤12)}, or a substituted version of any of these groups; or
a PEG moiety wherein the PEG moiety is of the formula: —(OCH₂CH₂)ₚOR₉; wherein:
p is 1-20; and
R₉ is hydrogen, alkyl_{(C≤8)}, or substituted alkyl_{(C≤8)}; or
an antitumor antibiotic linked through a cleavable covalent linker;
R₇ is hydrogen,
alkyl_{(C≤8)}, cycloalkyl_{(C≤8)}, alkenyl_{(C≤8)}, cycloalkenyl_{(C≤8)}, alkynyl_{(C≤8)}. alkoxy_{(C≤8)}, or a substituted version of any of these groups, or an amino protecting group;
X₁ and X₄ are each independently selected from: hydrogen, fluoride, alkyl_{(C1-6)}, or substituted alkyl_{(C1-6)}; or
X₂ and X₃ are each independently selected from: a PEG moiety wherein the PEG moiety is of the formula: —(OCH₂CH₂)ₙOR₈; wherein:
n is 1-20; and
R₈ is hydrogen, alkyl_{(C≤8)}, or substituted alkyl_{(C≤8)};
L₁ and L₂ are each independently absent, a neutral ligand, or an anionic ligand; and
M is a metal ion;
or a pharmaceutically acceptable salt thereof.

3. The compound of either claim 1 or claim 2 further defined as: 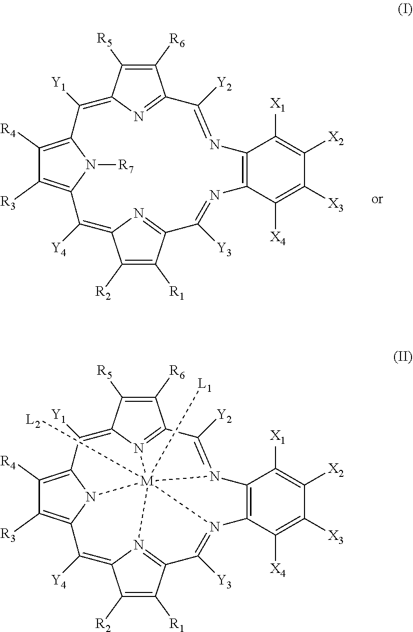
wnerein:
Y₁-Y₄ are each independently selected from hydrogen, alkyl_{(C1-6)}, or substituted alkyl_{(C1-6)};
R₁-R₆ are each independently selected from: hydrogen, amino, cyano, halo, hydroxy, hydroxyamino, or nitro,
alkyl_{(C≤12)}, cycloalkyl_{(C≤12)}, alkenyl_{(C≤12)}, cycloalkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, alkoxy_{(C≤12)}, acyloxy_{(C≤12)}, aryloxy _{(C≤12)}, heteroaryloxy _{(C≤12)}, heterocycloalkoxy_{(C≤12)}, amido_{(C≤12)}, alkylamino_{(C≤12)}, dialkylamino_{(C≤12)}, or a substituted version of any of these groups; or
a PEG moiety wherein the PEG moiety is of the formula: —(OCH₂CH₂)ₚOR₉; wherein:
p is 1-20; and
R₉ is hydrogen, alkyl_{(C≤8)}, or substituted alkyl_{(C≤8)}; or
an antitumor antibiotic linked through a cleavable covalent linker;
R₇ is hydrogen,
alkyl_{(C≤8)}, cycloalkyl_{(C≤8)}, alkenyl_{(C≤8)}, cycloalkenyl_{(C≤8)}, alkynyl_{(C≤8)}, alkoxy_{(C≤8)}, or a substituted version of any of these groups, or an amino protecting group;
X₁ and X₄ are each independently selected from: hydrogen, fluoride, alkyl_{(C1-6)}, or substituted alkyl_{(C1-6)}; or
X₂ and X₃ are each independently selected from: a PEG moiety wherein the PEG moiety is of the formula: —(OCH₂CH₂)ₙOR₈; wherein:
n is 1-20; and
R₈ is hydrogen, alkyl_{(C≤8)}, or substituted alkyl_{(C≤8)};
L₁ and L₂ are each independently absent, a neutral ligand, or an anionic ligand; and
M is a metal ion;
or a pharmaceutically acceptable salt thereof.

4. The compound according to any one of claims 1-3 further defined as: 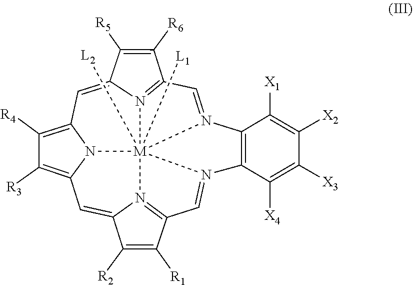
wherein:
R₁-R₆ are each independently selected from: hydrogen, amino, cyano, halo, hydroxy, hydroxyamino, or nitro,
alkyl_{(C≤12)}, cycloalkyl_{(C≤12)}, alkenyl_{(C≤12)}, cycloalkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, alkoxy_{(C≤12)}, acyloxy_{(C≤12)}, aryloxy _{(C≤12)}, heteroaryloxy _{(C≤12)}, heterocy cloalkoxy_{(C≤12)}, amido_{(C≤12)}, alkylamino_{(C≤12)}, dialkylamino_{(C≤12)}, or a substituted version of any of these groups; or
an antitumor antibiotic linked through a cleavable covalent linker;
X₁ and X₄ are each independently selected from: hydrogen, fluoride, alkyl_{(C1-6)}, or substituted alkyl_{(C1-6)}; or
X₂ and X₃ are each independently selected from: a PEG moiety wherein the PEG moiety is of the formula: —(OCH₂CH₂)ₙOR₈; wherein:
n is 1-20; and
R₈ is hydrogen, alkyl_{(C≤8)}, or substituted alkyl_{(C≤8)};
L₁ and L₂ are each independently absent, a neutral ligand, or an anionic ligand; and
M is a metal ion;
or a pharmaceutically acceptable salt thereof.

5. The compound according to any one of claims 1-4 further defined as: 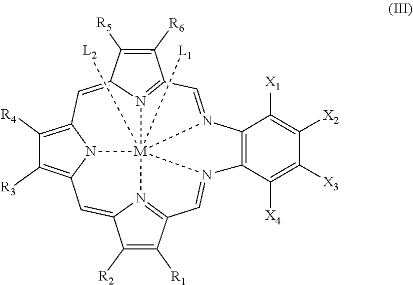
wherein:
R₁, R₂, R₅ and R₆ are each independently selected from hydrogen, alkyl_{(C≤12)}, or substituted alkyl_{(C≤12)}; or
an antitumor antibiotic linked through a cleavable covalent linker;
R₃ and R₄ are each independently selected from hydrogen, alkyl_{(C≤12)}, or substituted alkyl_{(C≤12)};
X₁ and X₄ are each independently selected from: hydrogen, fluoride, alkyl_{(C-1-6)}, or substituted alkyl_{(C1-6)}; or
X₂ and X₃ are each independently selected from: a PEG moiety wherein the PEG moiety is of the formula: —(OCH₂CH₂)ₙORₐ; wherein:
n is 1-20; and
R₈ is hydrogen, alkyl_{(C≤8)}, or substituted alkyl_{(C≤8)};
L₁ and L₂ are each independently absent, a neutral ligand, or an anionic ligand; and
M is a metal ion;
or a pharmaceutically acceptable salt thereof.

6. The compound according to any one of claims 1-5, wherein the antitumor antibiotic is an anthracycline antibiotic.

7. The compound of claim 6, wherein the antitumor antibiotic is further defined by the formula: 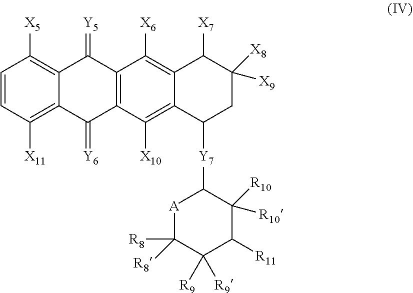
wherein:
X₅, X₆, X₇, X₁₀, and X₁₁ are each independently hydrogen, halo, hydroxy, carboxy, ester_{(C≤12)}, substituted ester_{(C≤12)}, alkoxy_{(C≤12)}, or substituted alkoxy_{(C≤12)};
X₈ is a covalent bond to the linker, acyl_{(C≤18)} or substituted acyl_{(C≤18)};
X₉ is hydrogen, hydroxy, alkoxy_{(C≤12)}, or substituted alkoxy_{(C≤12)};
Y₅, Y₆, and Y₇ are each independently O, S, or NH;
A is O or S; and
R₈, R₈′, R₉, R₉′, R₁₀, R₁₀′, and R₁₁ are each independently hydrogen, amino, halo, hydroxy, mercapto, or
alkyl_{(C≤8)}, alkoxy_{(C≤8)}, alkylthio_{(C≤8)}, alkylamino_{(C≤8)}, dialkylamino_{(C≤8)}, or a substituted version of any of these groups.

8. The compound of claim 7, wherein the antitumor antibiotic is further defined by the formula: 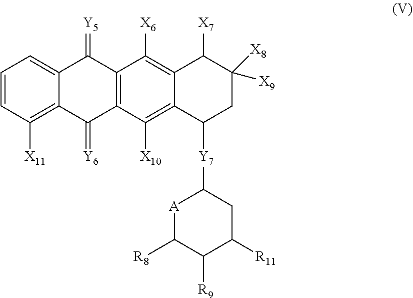
wherein:
X₆, X₇, X₁₀, and X₁₁ are each independently hydrogen, halo, hydroxy, carboxy, ester_{(C≤12)}, substituted ester_{(C≤12)}, alkoxy_{(C≤12)}, or substituted alkoxy_{(C≤12)};
X₈ is a covalent bond to the linker, acyl_{(C≤18)} or substituted acyl_{(C≤18)};
X₉ is hydrogen, hydroxy, alkoxy_{(C≤12)}, or substituted alkoxy_{(C≤12)};
Y₅, Y₆, and Y₇ are each independently O, S, or NH;
A is O or S; and
R₈, R₉, and R₁₁ are each independently hydrogen, amino, halo, hydroxy, mercapto, or
alkyl_{(C≤8)}, alkoxy_{(C≤8)}, alkylthio_{(C≤8)}, alkylamino_{(C≤8)}, dialkylamino_{(C≤8)}, or a substituted version of any of these groups.

9. The compound of claim 8, wherein the antitumor antibiotic is further defined by the formula: 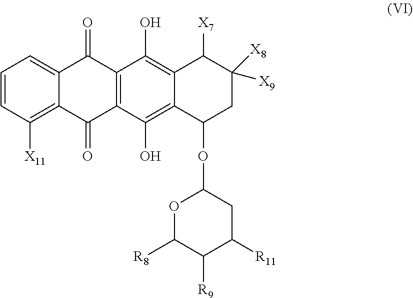
wherein:
X₇ and X₁₁ are each independently hydrogen, halo, hydroxy, carboxy, ester_{(C≤12)}, substituted ester_{(C≤12)}, alkoxy_{(C≤12)}, or substituted alkoxy_{(C≤12)};
X₈ is a covalent bond to the linker, acyl_{(C≤18)} or substituted acyl_{(C≤18)};
X₉ is hydrogen, hydroxy, alkoxy_{(C≤12)}, or substituted alkoxy_{(C≤12)}; and
R₈, R₉, and R₁₁ are each independently hydrogen, amino, halo, hydroxy, mercapto, or
alkyl_{(C≤8)}, alkoxy_{(C≤8)}, alkylthio_{(C≤8)}, alkylamino_{(C≤8)}, dialkylamino_{(C≤8)}, or a substituted version of any of these groups.

10. The compound according to any one of claims 1-9, wherein the antitumor antibiotic is doxorubicin, daunorubicin, epirubicin, idarubicin, pirarubicin, aclarubicin, or mitoxantrone.

11. The compound of claim 10, wherein the antitumor antibiotic is doxorubicin or daunorubicin.

12. The compound of claim 11, wherein the antitumor antibiotic is doxorubicin.

13. The compound according to any one of claims 1-12, wherein the antitumor antibiotic is linked to the texaphyrin core through a cleavable covalent linker, wherein the cleavable linker is a disulfide, a ketal, an acetal, a germinal dialcohol, an ester, a carbamate, a carbonate, an oxime, a hydrazone, or a peptide sequence which undergoes enzymatic cleavage.

14. The compound of claim 13, wherein the cleavable covalent linker is a disulfide, a ketal, an acetal, a germinal dialcohol, an ester, a carbamate, a carbonate, an oxime, or a hydrazone.

15. The compound of claim 14, wherein the cleavable covalent linker is a disulfide.

16. The compound of claim 14, wherein the cleavable covalent linker is a hydrazone.

17. The compound according to any one of claims 1-16, wherein the antitumor antibiotic linked through a cleavable covalent linker is further defined as:
-Y₅-A₁-Y₆-A₂-Y₇-A₃-
wherein:
Y₅, Y₆, and Y₇ are each independently selected from absent, alkanediyl_{(C≤12)}, alkenediyl_{(C≤12)}, arenediyl_{(C≤12)}, or a substituted version of any of these groups;
A₁ and A₃ are each independently selected from absent, —C(O)O—, —C(O)NH—, —OC(O)O—, —OC(O)NH—, —NHC(O)NH—, —C(NRₐ)O—, —C(NRₐ)NH—, —OC(NRₐ)O—, —OC(NRₐ)NH—, —NHC(NRₐ)NH—; wherein:
Rₐ is hydrogen, alkyl_{(C≤6)}, or substituted alkyl_{(C≤6)}; and
A₂ is a cleavable covalent linker.

18. The compound of claim 17, wherein Y₅ is alkanediyl_{(C1-8)} or substituted alkanediyl_{(C1-8)}.

19. The compound of claim 18, wherein Y₅ is —CH₂CH₂CH₂—.

20. The compound of claim 17, wherein Y₆ is alkanediyl_{(C1-8)} or substituted alkanediyl_{(C1-8)}.

21. The compound of claim 20, wherein Y₆ is —CH₂CH₂—.

22. The compound of claim 17, wherein Y₆ is absent.

23. The compound of claim 17, wherein Y₇ is alkanediyl_{(C1-8)} or substituted alkanediyl_{(C1-8)}.

24. The compound of claim 23, wherein Y₇ is —CH₂CH₂—.

25. The compound of claim 17, wherein Y₇ is absent.

26. The compound according to any one of claims 17-25, wherein A₁ is —OC(O)O—, —OC(O)NH—, or —NHC(O)NH—.

27. The compound of claim 26, wherein A₁ is —OC(O)NH—.

28. The compound according to any one of claims 17-27, wherein A₃ is —OC(O)O—, —OC(O)NH—, or —NHC(O)NH—.

29. The compound of claim 28, wherein A₃ is —OC(O)NH—.

30. The compound according to any one of claims 17-29, wherein A₂ is a cleavable covalent linker selected from a disulfide, a ketal, an acetal, a germinal dialcohol, an ester, a carbamate, a carbonate, an oxime, a hydrazone, and a peptide sequence which undergoes enzymatic cleavage.

31. The compound of claim 30, wherein A₂ is a peptide sequence which undergoes enzymatic cleavage.

32. The compound of claim 30, wherein A₂ is a disulfide, a ketal, an acetal, a germinal dialcohol, an ester, a carbamate, a carbonate, an oxime, or a hydrazone.

33. The compound of claim 32, wherein A₂ is a disulfide.

34. The compound of claim 32, wherein A₂ is a hydrazone.

35. The compound according to any one of claims 1-34, wherein R₁ and R₆ are alkyl_{(C1-6)} or substituted alkyl_{(C1-6)}.

36. The compound of claim 35, wherein R₁ and R₆ are methyl.

37. The compound according to any one of claims 1-36, wherein R₃ and R₄ are alkyl_{(C1-6)} or substituted alkyl_{(C1-6)}.

38. The compound of claim 37, wherein R₃ and R₄ are ethyl.

39. The compound according to any one of claims 1-38, wherein X₂ and X₃ are a PEG moiety of the formula: —(OCH₂CH₂)ₙOR₈; wherein:
n is 1-10; and
R₈ is alkyl_{(C≤8)} or substituted alkyl_{(C≤8)}.

40. The compound of claim 39, wherein X₂ and X₃ are a PEG moiety of the formula: —(OCH₂CH₂)ₙOR₈; wherein:
n is 1-5; and
R₈ is alkyl_{(C≤8)}.

41. The compound of claim 40, wherein X₂ and X₃ are —OCH₂CH₂OCH₂CH₂OCH₂CH₂OCH₃.

42. The compound according to any one of claims 1-41, wherein M is a gadolinium atom.

43. The compound of claim 42, wherein M is Gd(III).

44. The compound according to any one of claims 1-43, wherein L₁ and L₂ are anionic ligands.

45. The compound of claim 44, wherein L₁ and L₂ are acylate_{(C≤12)} or substituted acylate_{(C≤12)}.

46. The compound of claim 45, wherein L₁ and L₂ are acetate.

47. The compound according to any one of claims 1-46, wherein the compound is further defined as: 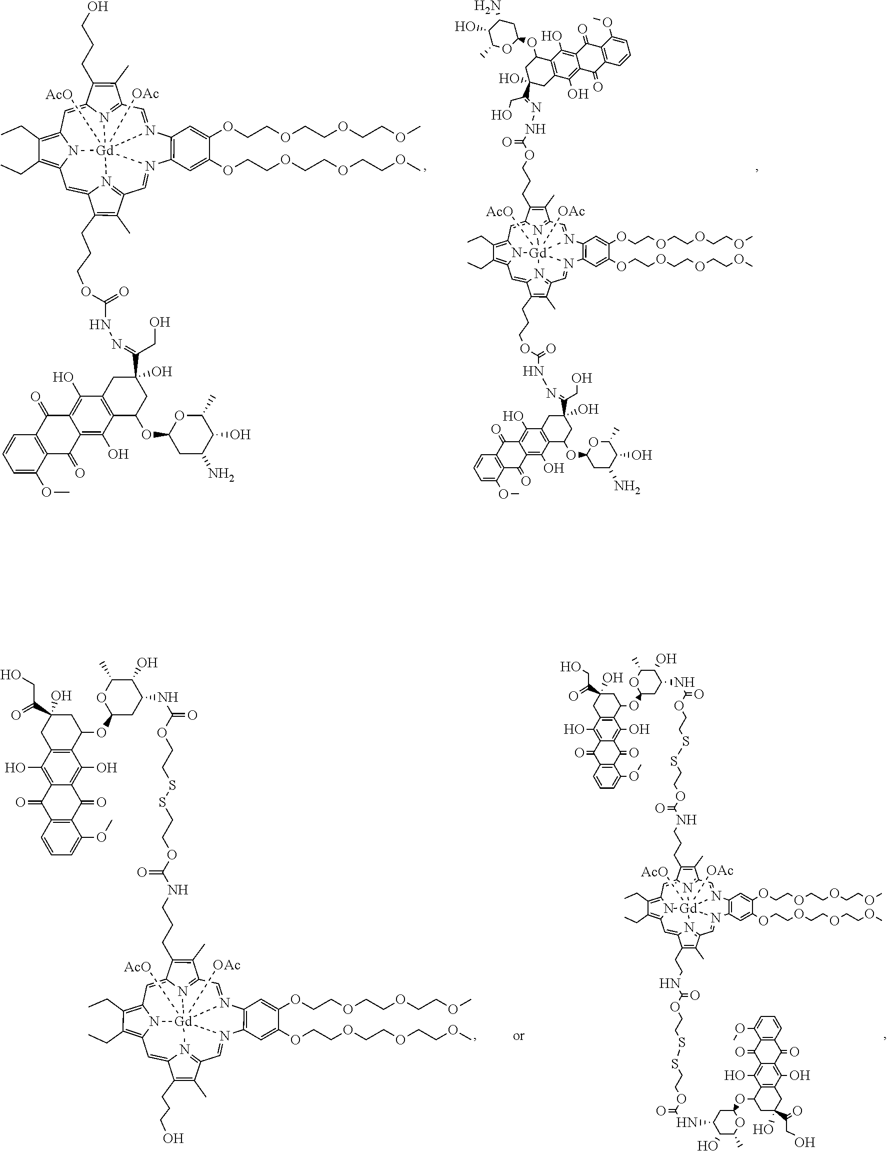
or a pharmaceutically acceptable salt thereof.

48. The compound of claim 47, wherein the compound is further defined as: 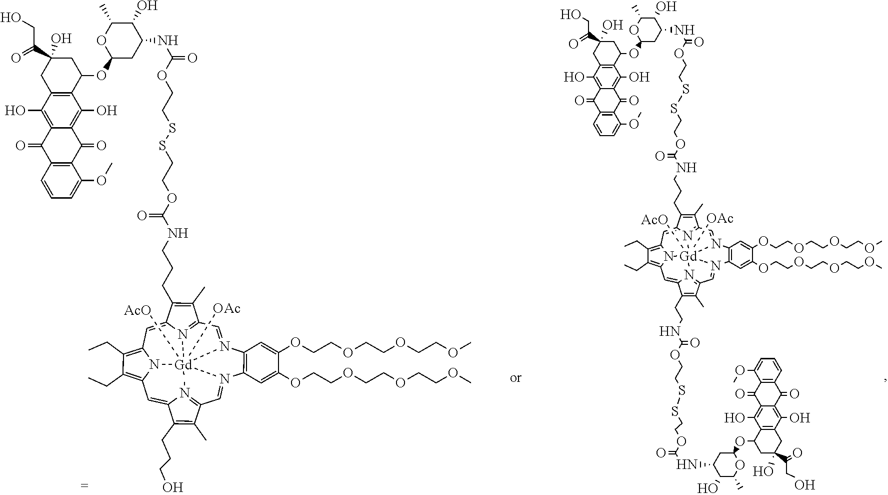
or a pharmaceutically acceptable salt thereof.

49. A pharmaceutical composition comprising:
(A) a compound according to any one of claims 1-48; and
(B) an excipient.

50. The pharmaceutical composition of claim 49, wherein the pharmaceutical composition is formulated for oral administration or administration by injection.

51. The pharmaceutical composition of claim 50, wherein the pharmaceutical composition is formulated for administration by injection.

52. The pharmaceutical composition of claim 51, wherein the pharmaceutical composition is formulated for intraarterial administration, intraperitoneal administration, or intravenous administration.

53. The pharmaceutical composition according to any one of claims 49-52, wherein the pharmaceutical composition is formulated as a liposome.

54. The pharmaceutical composition according to any one of claims 49-53, wherein the pharmaceutical composition is formulated as a unit dose.

55. A method of treating a disease or disorder in a patient in need thereof comprising administering to the patient a therapeutically effective amount of a compound or a composition according to any one of claims 1-54.

56. The method of claim 55, wherein the disease or disorder is cancer.

57. The method of claim 56, wherein the cancer is a carcinoma, sarcoma, lymphoma, leukemia, melanoma, mesothelioma, multiple myeloma, or seminoma.

58. The method of either claim 56 or claim 57, wherein the cancer is of the bladder, blood, bone, brain, breast, central nervous system, cervix, colon, endometrium, esophagus, gall bladder, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid.

59. The method according to any one of claims 56-58, wherein the cancer is leukemia, Hodgkin's lymphoma, bladder cancer, breast cancer, colon cancer, stomach cancer, lung cancer, liver cancer, ovarian cancer, a sarcoma of the soft tissue, or multiple myeloma.

60. The method of claim 59, wherein the cancer is colon cancer, liver cancer, or lung cancer.

61. The method according to any one of claims 56-60, wherein the method further comprises administering a second anti-cancer therapy.

62. The method of claim 61, wherein the second anti-cancer therapy is another chemotherapeutic drug, surgery, radiotherapy, photodynamic therapy, sonodynamic therapy, cryotherapy, or immunotherapy.

63. The method according to any one of claims 55-62, wherein the compound or composition is administered once.

64. The method according to any one of claims 55-62, wherein the compound or composition is administered two or more times.

65. A method of imaging a patient comprising:
(A) administering the compound or pharmaceutical composition according to any one of claims 1-54; and
(B) imaging the patient to determine the presence of a tumor.

66. The method of claim 65, wherein the patient is imaged using MRI, CT, SPECT, SPECT/MRI, or SPECT/CT.

67. The method of either claim 65 or claim 66, wherein the tumor is cancer.

68. The method of claim 67, wherein the tumor is a carcinoma.
